(19) United States
(12) Patent Application Publication
(10) Pub. No.: US 2017/0202752 A1
(43) Pub. Date: Jul. 20, 2017

(54) PROTEIN-REPELLENT DENTAL MATERIALS AND USE THEREOF IN DENTAL APPLICATIONS

(71) Applicants: Huakun XU, Frederick, MD (US); Ning ZHANG, Baltimore, MD (US)

(72) Inventors: Huakun XU, Frederick, MD (US); Ning ZHANG, Baltimore, MD (US)

(73) Assignee: University of Maryland, Baltimore, Baltimore, MD (US)

(21) Appl. No.: 15/328,780

(22) PCT Filed: Jul. 24, 2015
(86) PCT No.: PCT/US15/42103

§ 371 Date: Jan. 24, 2017

Related U.S. Application Data
(60) Provisional application No. 62/029,184, filed on Jul. 25, 2014.

(51) Int. Cl. A61K 6/00; A61K 31/685

(52) U.S. Cl. CPC A61K 6/0067; A61K 6/0017; A61K 6/007; A61K 6/0029; A61K 31/685

(57) ABSTRACT

The present invention provides protein-repellent dental materials that can be widely applied in a variety of dental applications. The protein-repellent dental materials of the invention include dental primers, dental adhesives, dental resins, dental composites, dental bonding systems and the like, as well as dental cements, dental sealants, dental bases and dental liners, each of which is protein-repellent and each of which comprises a protein-repellent agent. In certain aspects, the protein-repellent agent is 2-methacryloyloxyethyl phosphorylcholine (MPC).

### BACKGROUND

Tooth caries are the result of a dietary carbohydrate-modified bacterial infectious disease, one of the most common bacterial infections in humans (Loesche, 1986; van Houte, 1994; Featherstone, 2000). The basic mechanism of dental caries is demineralization, or mineral loss, through attack by acid generated by bacteria (Featherstone, 2004; Deng, 2005; Totiam et al., 2007). Therefore, acidogenic bacteria growth, typically in the context of plaque and biofilm formation, is responsible for dental caries (Loesche, 1986; van Houte, 1994; Zero, 1995; Featherstone, 2000; Deng et al., 2005; Cenci et al., 2009).

Plaque formation has been described to have three steps: pellicle formation, bacteria colonization, and biofilm maturation (Burne, 1998). In the initial stage, a proteinaceous film called pellicle forms on the tooth surface with adsorbed components from saliva, mucosa, and bacteria (Carlen et al., 2001). Bacteria then adhere and colonize on this surface to grow into a biofilm, which is a heterogeneous structure consisting of clusters of various types of bacteria embedded in an extracellular matrix (Stoodley et al., 2008). Cariogenic bacteria such as Streptococcus mutans (S. mutans) and lactobacilli in the plaque can take nutrients from carbohydrates and produce organic acids. Acid production causes demineralization to the tooth structure beneath the biofilm.

Resin composites have been increasingly used for tooth cavity restorations because of their aesthetics, direct-filling capability, and enhanced performance (Ferracane, 1995; Bayne et al., 1998; Lim et al., 2002; Ruddell et al., 2002; Watts et al., 2003; Drummond, 2008). While there has been significant improvement in resin compositions, filler types, and cure conditions since their introduction (Ruddell et al., 2002; Imazato, 2003; Drummond and Bapna, 2003; Watts et al., 2003; Lu et al., 2005; Xu X et al., 2006; Kramer et al., 2006), formation of secondary caries and bulk fracture remain challenges to the use of resins (Sarrett, 2005; Sakaguchi, 2005). Resin composites generally do not prevent secondary caries because they do not hinder bacteria colonization and plaque formation. In fact, several studies have indicated that resin composites have a greater accumulation of bacteria and plaque than other restorative materials (Svanberg et al., 1990; Imazato et al., 1994; Takahashi et al., 2004). Indeed, caries at the restoration margins are a frequent reason for replacing existing restorations (Mjör et al., 2000), accounting for 50-70% of all restorations (Deligeorgi et al., 2001; Frost, 2002). Also, secondary caries may form in the tooth-restoration interface. Dental bonding systems are used to adhere resin composites to tooth structures (Park et al., 2009), but microleakage can allow bacteria to invade the interface. Residual bacteria can also exist in a clean tooth cavity prior to being packed with the resin composition.

Use of dental materials, such as resin composites, that passively inhibit plaque and biofilm formation could be one means to prevent the development of tooth caries. In the oral environment with salivary flow, dental structures become coated with a salivary pellicle that comprises of a layer of selectively adsorbed salivary proteins (Lendenmann et al., 2000). Attachment of oral bacteria follows, and in turn, biofilms form on the structures (Kolenbrander and London, 1993; Donlan and Costerton, 2002). If the initial attachment of salivary proteins to dental structures could be inhibited, attachment of oral bacteria could be slowed, and in turn biofilm formation prevented (Kolenbrander and London, 1993; Donlan and Costerton, 2002).

The present invention is directed to the development dental materials that can repel proteins and to other important ends.

### SUMMARY

Disclosed herein are protein-repellent dental materials that can be widely applied in a variety of dental applications. The protein-repellent dental materials of the invention include dental primers, dental adhesives, dental resins, dental composites, dental bonding systems and the like, as well as dental cements, dental sealants, dental bases and dental liners, each of which is protein-repellent and each of which comprises a protein-repellent agent. In certain aspects, the protein-repellent agent is 2-methacryloyloxyethyl phosphorylcholine (MPC).

The protein-repellent technology can be combined with antibacterial and remineralization agents, methods and compositions (for example, but not limited to, the antibacterial and remineralization agents, methods and compositions described in WO2012/003290, WO2013/119901, and WO2014/062347) for use in the dental primers, adhesives, resins, composites, bonding systems, cements, sealants, bases and liners mentioned above.

While the protein-repellent dental materials described herein can be used in a wide variety of products and applications, one of the most common applications is the use of the materials in dental bonding systems. Such systems are used where a bond between two components is required in a dental application. For example, bonding systems can be prepared for use in dentin bonding, enamel bonding, coating tooth roots, marginal repair, as a crown cement, as an inlay/onlay cement, as a pit and fissure sealant, and as an orthodontic bracket adhesive or cement. As a specific example, dental bonding systems can be used to secure resin composites filling voids in teeth after removal of decayed materials.

Thus, and in a first embodiment, the present invention includes protein-repellent dental bonding systems. These systems comprise dental primers, dental adhesives and optionally an etchant, wherein the dental primer, the dental adhesive, or both the dental primer and the dental adhesive comprise a protein-repellent agent. The protein-repellent dental bonding systems include a three-component system comprising a dental primer, a dental adhesive and an etchant; a two-component system comprising a dental primer and a dental adhesive; a three-step bonding system; a two-step bonding system; and a one-step self-adhesive bonding system.

In one aspect of this embodiment, the dental bonding system is a three-component protein-repellent dental bonding system comprising (i) an etchant, (ii) a dental primer, and (iii) a dental adhesive, wherein the dental primer, the dental adhesive, or both the dental primer and the dental adhesive comprise a protein-repellent agent. In certain aspects, the primer may further comprise an antibacterial agent, a remineralizing agent, or both. In certain aspects, the adhesive may further comprise an antibacterial agent, a remineralizing agent, or both.

In one aspect of this embodiment, the dental bonding system is a two-component protein-repellent dental bonding system comprising (i) a dental primer and (ii) a dental adhesive, wherein the dental primer, the dental adhesive, or both the dental primer and the dental adhesive comprise a protein-repellent agent. In certain aspects, the primer may further comprise an antibacterial agent, a remineralizing agent, or both. In certain aspects, the adhesive may further comprise an antibacterial agent, a remineralizing agent, or both.

In one aspect of this embodiment, the dental bonding system is a three-step protein-repellent dental bonding system comprising (i) an etchant, (ii) a dental primer, and (iii) a dental adhesive, wherein the dental primer, the dental adhesive, or both the dental primer and the dental adhesive comprise a protein-repellent agent. In certain aspects, the primer may further comprise an antibacterial agent, a remineralizing agent, or both. In certain aspects, the adhesive may further comprise an antibacterial agent, a remineralizing agent, or both.

In one aspect of this embodiment, the dental bonding system is a two-step protein-repellent dental bonding system comprising (i) an etchant, and (ii) a mixture comprising a dental primer and a dental adhesive, wherein the dental primer, the dental adhesive, or both the dental primer and the dental adhesive comprise a protein-repellent agent. In certain aspects, the primer may further comprise an antibacterial agent, a remineralizing agent, or both. In certain aspects, the adhesive may further comprise an antibacterial agent, a remineralizing agent, or both.

In one aspect of this embodiment, the dental bonding system is a one-step self-adhesive protein-repellent bonding system comprising a mixture that comprises an etchant, a dental primer, and a dental adhesive, wherein the dental primer, the dental adhesive, or both the dental primer and the dental adhesive comprise a protein-repellent agent. In certain aspects, the primer may further comprise an antibacterial agent, a remineralizing agent, or both. In certain aspects, the adhesive may further comprise an antibacterial agent, a remineralizing agent, or both.

The dental primer may be a single primer or a mixture of two or more primers. The primers include, but are not limited to, those comprising Bisphenol A diglycidyl methacrylate (Bis-GMA), glycerol dimethacrylate (GDMA), 2-hydroxyethyl methacrylate (HEMA), mono-2-methacryloyloxyethyl phthalate (MMEP), methacrylic acid (MA), methyl methacrylate (MMA), 4-acryloyloxyethyl trimellitate anhydride (4-AETA), N-phenylglycine glycidyl methacrylate (NPG-GMA), N-tolylglycine glycidyl methacrylate or N-(2-hydroxy-3-((2-methyl-1-oxo-2-propenyl)oxy)propyl)-N-tolyl glycine (NTG-GMA), pyromellitic diethylmethacrylate or 2,5-dimethacryloyloxyethyloxycarbonyl-1,4-benzenedicarboxylic acid (PMDM), pyromellitic glycerol dimethacrylate or 2,5-bis(1,3-dimethacryloyloxyprop-2-yloxycarbonyl)benzene-1,4-dicarboxylic acid (PMGDM), and triethylene glycol dimethacrylate (TEGDMA).

In certain aspects, the dental primer is SCOTCHBOND MULTI-PURPOSE™ (SBMP) primer comprising 35-45% 2-hydroxyethylmethacrylate (HEMA), 10-20% copolymer of acrylic and itaconic acids, 40-50% water. In certain other aspects, the dental primer comprises PMGDM/HEMA at 3.3/1 ratio+1% BAPO+50% acetone.

The dental adhesive may be a single adhesive or a mixture of two or more adhesives. The adhesives include, but are not limited to, those comprising ethoxylated bisphenol A glycol dimethacrylate (Bis-EMA), bisphenol A diglycidyl methacrylate (Bis-GMA), 2-hydroxyethyl methacrylate (HEMA), triethylene glycol dimethacrylate (TEGDMA), urethane dimethacrylate (UDMA), 4-methacryloyloxyethyl trimellitate anhydride (4-META), methacrylic acid (MA), methyl methacrylate (MMA), 4-acryloyloxyethyl trimellitate anhydride (4-AETA), ethyleneglycol dimethacrylate (EGDMA), glycerol dimethacrylate (GDMA), glycerol phosphate dimethacrylate (GPDM), pyromellitic glycerol dimethacrylate or 2,5-bis(1,3-dimethacryloyloxyprop-2-yloxycarbonyl)benzene-1,4-dicarboxylic acid (PMGDM).

In certain aspects, the dental adhesive is SBMP adhesive comprising 60-70% BisGMA and 30-40% HEMA. In certain other aspects, the dental adhesive comprises BisGMA/TEGMA at 7/3 ratio+1% BAPO.

The etchant is one or more etchants including, but not limited to, 37% phosphoric acid.

In each aspect of this embodiment, the protein-repellant agent may be a single protein-repellant agent or a mixture of two or more protein-repellant agents. Suitable protein-repellant agents include 2-methacryloyloxyethyl phosphorylcholine (MPC), poly(hydroxyethyl methacrylate) (HEMA) and derivatives thereof, and poly(N-isopropylacrylamide) and derivatives thereof.

When the dental primer comprises a protein-repellant agent, the amount of protein-repellant agent in the dental primer ranges from about 0.5% to 50% of the mass of the dental primer. In certain aspects, the range is from about 1% to 25%, about 2.5% to 20%, about 4% to 15%, or about 5% to 12.5% of the mass of the dental primer. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10% of the mass of the dental primer.

When the dental adhesive comprises a protein-repellant agent, the amount of protein-repellant agent in the dental adhesive ranges from about 0.5% to 50% of the mass of the dental adhesive. In certain aspects, the range is from about 1% to 25%, about 2.5% to 20%, about 4% to 15%, or about 5% to 12.5% of the mass of the dental adhesive. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10% of the mass of the dental adhesive.

When present, the antibacterial agent may be a single antibacterial agent or a mixture of two or more antibacterial agents. The antibacterial agents include, but are not limited to, antibacterial monomers, silver-containing nanoparticles (NAg), quaternary ammonium salts (QAS), chlorhexidine particles, TiO₂ particles and ZnO particles.

The antibacterial monomers include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). In certain aspects, the antibacterial monomer is DMADDM, DMATDM, DMATTDM, DMAPDM or DMAHDM.

When used, the amount of antibacterial monomers present in the dental primer or dental adhesive independently ranges from about 0.5% to 50% of the mass of the dental primer or dental adhesive. In certain aspects, the range is from about 1% to 25%, about 1.5% to 20%, about 2% to 15%, or about 2.5% to 12.5% of the mass of the dental primer or dental adhesive. In certain aspects, the amount of antibacterial monomers is about 2.5%, 3%, 3.5%, 4%, 4.5%, 5%, 5.5%, 6%, 6.5%, 7%, or 7.5% of the mass of the dental primer or dental adhesive.

When used, the amount of quaternary ammonium salts present in the dental primer or dental adhesive independently ranges from about 1% to 30% of the mass of the dental primer or dental adhesive, preferably about 7.5% to 15%.

When used, the amount of silver-containing nanoparticles, chlorhexidine particles, TiO2 particles or ZnO particles present in the dental primer or dental adhesive independently ranges from about 0.01% and 20% of the mass of the dental primer or dental adhesive, preferably about 0.08% to 10% of the mass of the dental primer or dental adhesive.

When present, the remineralizing agent may be a single remineralizing agent or a mixture of two or more remineralizing agents. The remineralizing agents include, but are not limited to, nanoparticles of amorphous calcium phosphate (NACP).

When present, the amount of NACP included in the dental primer or dental adhesive independently ranges from about 10% to about 40% of the mass of the dental primer or dental adhesive. In particular aspects, the mass fraction of NACP in the dental primer or dental adhesive is about 25%, 30% or 35% of the mass of the dental primer or dental adhesive.

In a particular aspect of the dental binding systems of the invention, the dental primer comprises about 82.5-92.5% dental primer, about 5-10% MPC, and about 2.5-7.5% antibacterial agent by mass of the dental primer, wherein the antibacterial agent is DMADDM, DMAPDM, or DMAHDM. In this same aspect, the dental adhesive comprises about 82.5-92.5% dental adhesive, about 5-10% MPC, and about 2.5-7.5% antibacterial agent by mass of the dental adhesive, wherein the antibacterial agent is DMADDM, DMAPDM, or DMAHDM.

In a particular aspect of the dental binding systems of the invention, the dental primer comprises about 85-90% dental primer, about 5-10% MPC, and about 5% antibacterial agent by mass of the dental primer, wherein the antibacterial agent is DMADDM, DMAPDM, or DMAHDM. In this same aspect, the dental adhesive comprises about 85-90% dental adhesive, about 5-10% MPC, and about 5% antibacterial agent by mass of the dental adhesive, wherein the antibacterial agent is DMADDM, DMAPDM, or DMAHDM.

In a particular aspect of the dental binding systems of the invention, the dental primer comprises about 85-90% dental primer, about 7.5% MPC, and about 2.5-7.5% antibacterial agent by mass of the dental primer, wherein the antibacterial agent is DMADDM, DMAPDM, or DMAHDM. In this same aspect, the dental adhesive comprises about 85-90% dental adhesive, about 7.5% MPC, and about 2.5-7.5% antibacterial agent by mass of the dental adhesive, wherein the antibacterial agent is DMADDM, DMAPDM, or DMAHDM.

In a particular aspect of the dental binding systems of the invention, the dental primer comprises about 87.5% dental primer, about 7.5% MPC, and about 5% DMAHDM by mass of the dental primer. In this same aspect, the dental adhesive comprises about 87.5% dental adhesive, about 7.5% MPC, and about 5% DMAHDM by mass of the dental adhesive.

In a particular aspect of the dental binding systems of the invention, the dental primer comprises about 90-95% dental primer and about 5-10% MPC by mass of the dental primer. In this same aspect, the dental adhesive comprises about 90-95% dental adhesive and about 5-10% MPC by mass of the dental adhesive.

In a particular aspect of the dental binding systems of the invention, the dental primer comprises about 92.5% dental primer and about 7.5% MPC by mass of the dental primer. In this same aspect, the dental adhesive comprises about 92.5% dental adhesive and about 7.5% MPC by mass of the dental adhesive.

### Protein-Repellant Dental Primer

As will be apparent from the description above, and in a second embodiment, the invention includes a protein-repellant dental primer comprising a dental primer and a protein-repellant agent. In certain aspects, the protein-repellant dental primer may further comprise an antibacterial agent, a remineralizing agent, or both.

The dental primer may be a single primer or a mixture of two or more primers. The primers include, but are not limited to, those comprising Bisphenol A diglycidyl methacrylate (Bis-GMA), glycerol dimethacrylate (GDMA), 2-hydroxyethyl methacrylate (HEMA), mono-2-methacryloyloxyethyl phthalate (MMEP), methacrylic acid (MA), methyl methacrylate (MMA), 4-acryloyloxyethyl trimellitate anhydride (4-AETA), N-phenylglycine glycidyl methacrylate (NPG-GMA), N-tolylglycine glycidyl methacrylate or N-(2-hydroxy-3-((2-methyl-1-oxo-2-propenyl)oxy)propyl)-N-tolyl glycine (NTG-GMA), pyromellitic diethylmethacrylate or 2,5-dimethacryloyloxyethyloxycarbonyl-1,4-benzenedicarboxylic acid (PMDM), pyromellitic glycerol dimethacrylate or 2,5-bis(1,3-dimethacryloyloxyprop-2-yloxycarbonyl)benzene-1,4-dicarboxylic acid (PMGDM), and triethylene glycol dimethacrylate (TEGDMA).

In certain aspects, the dental primer is SCOTCHBOND MULTI-PURPOSE™ (SBMP) primer comprising 35-45% 2-hydroxyethylmethacrylate (HEMA), 10-20% copolymer of acrylic and itaconic acids, 40-50% water. In certain other aspects, the dental primer comprises PMGDM/HEMA at 3.3/1 ratio+1% BAPO+50% acetone.

In each aspect of this embodiment, the protein-repellant agent may be a single protein-repellant agent or a mixture of two or more protein-repellant agents. Suitable protein-repellant agents include 2-methacryloyloxyethyl phosphorylcholine (MPC), poly(hydroxyethyl methacrylate) (HEMA) and derivatives thereof, and poly(N-isopropylacrylamide) and derivatives thereof.

The amount of protein-repellant agent in the protein-repellant dental primer ranges from about 0.5% to 50% of the mass of the dental primer. In certain aspects, the range is from about 1% to 25%, about 2.5% to 20%, about 4% to 15%, or about 5% to 12.5% of the mass of the dental primer. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10%, of the mass of the dental primer.

When present, the antibacterial agent may be a single antibacterial agent or a mixture of two or more antibacterial agents. The antibacterial agents include, but are not limited to, antibacterial monomers, silver-containing nanoparticles (NAg), quaternary ammonium salts (QAS), chlorhexidine particles, TiO₂ particles and ZnO particles.

The antibacterial monomers include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). In certain aspects, the antibacterial monomer is DMADDM, DMATDM, DMATTDM, DMAPDM or DMAHDM.

When used, the amount of antibacterial monomers present in the dental primer ranges from about 0.5% to 50% of the mass of the dental primer. In certain aspects, the range is from about 1% to 25%, about 1.5% to 20%, about 2% to 15%, or about 2.5% to 12.5% of the mass of the dental primer. In certain aspects, the amount of antibacterial monomers is about 2.5%, 3%, 3.5%, 4%, 4.5%, 5%, 5.5%, 6%, 6.5%, 7%, or 7.5% of the mass of the dental primer.

When used, the amount of quaternary ammonium salts present in the protein-repellant dental primer ranges from about 1% to 30% of the mass of the dental primer, preferably about 7.5% to 15%.

When used, the amount of silver-containing nanoparticles, chlorhexidine particles, TiO2 particles or ZnO particles present in the protein-repellant dental primer ranges from about 0.01% and 20% of the mass of the dental primer, preferably about 0.08% to 10% of the mass of the dental primer.

When present, the remineralizing agent may be a single remineralizing agent or a mixture of two or more remineralizing agents. The remineralizing agents include, but are not limited to, nanoparticles of amorphous calcium phosphate (NACP).

When present, the amount of amount of NACP included in the protein-repellant dental primer ranges from about 10% to 40% of the mass of the dental primer. In particular aspects, the mass fraction of NACP in the dental primer is about 25%, 30% or 35% of the mass of the dental primer.

In a particular aspect, the present invention is directed to a protein-repellant dental primer comprising a dental primer, MPC and an antibacterial agent, wherein MPC is present in an amount ranging from about 5-10% by mass of the dental primer, wherein the antibacterial agent is present in an amount ranging from about 2.5-7.5% by mass of the dental primer, and wherein the antibacterial agent is DMADDM, DMAPDM, or DMAHDM.

In a particular aspect, the present invention is directed to a protein-repellant dental primer comprising a dental primer, MPC and an antibacterial agent, wherein MPC is present in an amount ranging from about 5-10% by mass of the dental primer, wherein the antibacterial agent is present in an amount of about 5% by mass of the dental primer, and wherein the antibacterial agent is DMADDM, DMAPDM, or DMAHDM.

In a particular aspect, the present invention is directed to a protein-repellant dental primer comprising a dental primer, MPC and an antibacterial agent, wherein MPC is present in an amount of about 7.5% by mass of the dental primer, wherein the antibacterial agent is present in an amount ranging from about 2.5-7.5% by mass of the dental primer, and wherein the antibacterial agent is DMADDM, DMAPDM, or DMAHDM.

In a particular aspect, the present invention is directed to a protein-repellant dental primer comprising a dental primer, MPC and an antibacterial agent, wherein MPC is present in an amount of about 7.5% by mass of the dental primer, wherein the antibacterial agent is present in an amount of about 5% by mass of the dental primer, and wherein the antibacterial agent is DMADDM, DMAPDM, or DMAHDM.

In a particular aspect, the present invention is directed to a protein-repellant dental primer comprising a dental primer and MPC, wherein MPC is present in an amount ranging from about 5-10% by mass of the dental primer.

In a particular aspect, the present invention is directed to a protein-repellant dental primer comprising a dental primer and MPC, wherein MPC is present in an amount of about 7.5% by mass of the dental primer.

### Protein-Repellant Dental Adhesive

As will be further apparent from the description above, and in a third embodiment, the invention includes a protein-repellant dental adhesive comprising a dental adhesive and a protein-repellant agent. In certain aspects, the protein-repellant dental adhesive may further comprise an antibacterial agent, a remineralizing agent, or both.

The dental adhesive may be a single adhesive or a mixture of two or more adhesives. The adhesives include, but are not limited to, those comprising ethoxylated bisphenol A glycol dimethacrylate (Bis-EMA), bisphenol A diglycidyl methacrylate (Bis-GMA), 2-hydroxyethyl methacrylate (HEMA), triethylene glycol dimethacrylate (TEGDMA), urethane dimethacrylate (UDMA), 4-methacryloyloxyethyl trimellitate anhydride (4-META), methacrylic acid (MA), methyl methacrylate (MMA), 4-acryloyloxyethyl trimellitate anhydride (4-AETA), ethyleneglycol dimethacrylate (EGDMA), glycerol dimethacrylate (GDMA), glycerol phosphate dimethacrylate (GPDM), pyromellitic glycerol dimethacrylate or 2,5-bis(1,3-dimethacryloyloxyprop-2-yloxycarbonyl)benzene-1,4-dicarboxylic acid (PMGDM).

In certain aspects, the dental adhesive is SBMP adhesive comprising 60-70% BisGMA and 30-40% HEMA. In certain other aspects, the dental adhesive comprises BisGMA/TEGMA at 7/3 ratio+1% BAPO.

In each aspect of this embodiment, the protein-repellant agent may be a single protein-repellant agent or a mixture of two or more protein-repellant agents. Suitable protein-repellant agents include 2-methacryloyloxyethyl phosphorylcholine (MPC), poly(hydroxyethyl methacrylate) (HEMA) and derivatives thereof, and poly(N-isopropylacrylamide) and derivatives thereof.

The amount of protein-repellant agent in the protein-repellant dental adhesive ranges from about 0.5% to 50% of the mass of the dental adhesive. In certain aspects, the range is from about 1% to 25%, about 2.5% to 20%, about 4% to 15%, or about 5% to 12.5% of the mass of the dental adhesive. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10%, of the mass of the dental adhesive.

When present, the antibacterial agent may be a single antibacterial agent or a mixture of two or more antibacterial agents. The antibacterial agents include, but are not limited to, antibacterial monomers, silver-containing nanoparticles (NAg), quaternary ammonium salts (QAS), chlorhexidine particles, TiO₂ particles and ZnO particles.

The antibacterial monomers include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). In certain aspects, the antibacterial monomer is DMADDM, DMATDM, DMATTDM, DMAPDM or DMAHDM.

When used, the amount of antibacterial monomers present in the protein-repellant dental adhesive ranges from about 0.5% to 50% of the mass of the dental adhesive. In certain aspects, the range is from about 1% to 25%, about 1.5% to 20%, about 2% to 15%, or about 2.5% to 12.5% of the mass of the dental adhesive. In certain aspects, the amount of antibacterial monomers is about 2.5%, 3%, 3.5%, 4%, 4.5%, 5%, 5.5%, 6%, 6.5%, 7%, or 7.5% of the mass of the dental adhesive.

When used, the amount of quaternary ammonium salts present in the protein-repellant dental adhesive ranges from about 1% to 30% of the mass of the dental adhesive, preferably about 7.5% to 15%.

When used, the amount of silver-containing nanoparticles, chlorhexidine particles, TiO2 particles or ZnO particles present in the protein-repellant dental adhesive ranges from about 0.01% and 20% of the mass of the dental adhesive, preferably about 0.08% to 10% of the mass of the dental adhesive.

When present, the remineralizing agent may be a single remineralizing agent or a mixture of two or more remineralizing agents. The remineralizing agents include, but are not limited to, nanoparticles of amorphous calcium phosphate (NACP).

When present, the amount of amount of NACP included in the protein-repellant dental adhesive ranges from about 10% to 40% of the mass of the dental adhesive. In particular aspects, the mass fraction of NACP in the dental adhesive is about 25%, about 30% or about 35% of the mass of the dental adhesive.

In a particular aspect, the present invention is directed to a protein-repellant dental adhesive comprising a dental adhesive, MPC and an antibacterial agent, wherein MPC is present in an amount ranging from about 5-10% by mass of the dental adhesive, wherein the antibacterial agent is present in an amount ranging from about 2.5-7.5% by mass of the dental adhesive, and wherein the antibacterial agent is DMADDM, DMAPDM, or DMAHDM.

In a particular aspect, the present invention is directed to a protein-repellant dental adhesive comprising a dental adhesive, MPC and an antibacterial agent, wherein MPC is present in an amount ranging from about 5-10% by mass of the dental adhesive, wherein the antibacterial agent is present in an amount of about 5% by mass of the dental adhesive, and wherein the antibacterial agent is DMADDM, DMAPDM, or DMAHDM.

In a particular aspect, the present invention is directed to a protein-repellant dental adhesive comprising a dental adhesive, MPC and an antibacterial agent, wherein MPC is present in an amount of about 7.5% by mass of the dental adhesive, wherein the antibacterial agent is present in an amount ranging from about 2.5-7.5% by mass of the dental adhesive, and wherein the antibacterial agent is DMADDM, DMAPDM, or DMAHDM.

In a particular aspect, the present invention is directed to a protein-repellant dental adhesive comprising a dental adhesive, MPC and an antibacterial agent, wherein MPC is present in an amount of about 7.5% by mass of the dental adhesive, wherein the antibacterial agent is present in an amount of about 5% by mass of the dental adhesive, and wherein the antibacterial agent is DMADDM, DMAPDM, or DMAHDM.

In a particular aspect, the present invention is directed to a protein-repellant dental adhesive comprising a dental adhesive and MPC, wherein MPC is present in an amount ranging from about 5-10% by mass of the dental adhesive.

In a particular aspect, the present invention is directed to a protein-repellant dental adhesive comprising a dental adhesive and MPC, wherein MPC is present in an amount of about 7.5% by mass of the dental adhesive.

### Protein-Repellant Dental Resin

The protein-repellant dental materials of the invention include dental resins. Thus, and in a fourth embodiment, the invention includes a protein-repellant dental resin comprising a dental resin and a protein-repellant agent. In certain aspects, the protein-repellant dental resin may further comprise an antibacterial agent, a remineralizing agent, or both.

The resin in the protein-repellant dental resin is one or more resins selected from the group consisting of bis-GMA (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer, and a heat-cured polymer. In certain aspects, the resin is a 1:1 mass ratio of bis-GMA and TEGDMA.

In each aspect of this embodiment, the protein-repellant agent may be a single protein-repellant agent or a mixture of two or more protein-repellant agents. Suitable protein-repellant agents include 2-methacryloyloxyethyl phosphorylcholine (MPC), poly(hydroxyethyl methacrylate) (HEMA) and derivatives thereof, and poly(N-isopropylacrylamide) and derivatives thereof.

The amount of protein-repellant agent in the protein-repellant dental resin ranges from about 0.5% to 30% of the mass of the dental resin. In certain aspects, the range is from about 1% to 25%, about 1.5% to 20%, about 2% to 15%, or about 2.5% to 10% of the mass of the dental resin. In certain aspects, the amount of protein-repellant agent is about 0.5%, 1.0%, 1.5%, 2.0%, 2.5%, 3%, 3.5%, 4%, 4.5%, 5%, 5.5%, 6%, 6.5%, 7%, 7.5%, 8%, 8.5%, 9%, 9.5%, or 10%, of the mass of the dental resin.

When present, the antibacterial agent may be a single antibacterial agent or a mixture of two or more antibacterial agents. The antibacterial agents include, but are not limited to, antibacterial monomers, silver-containing nanoparticles (NAg), quaternary ammonium salts (QAS), chlorhexidine particles, TiO₂ particles and ZnO particles.

The antibacterial monomers include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). In certain aspects, the antibacterial monomer is DMADDM, DMATDM, DMATTDM, DMAPDM or DMAHDM.

When used, the amount of antibacterial monomers present in the protein-repellant dental resin ranges from about 0.1% to 15% of the mass of the dental resin. In certain aspects, the range is from about 0.5% to 10%, about 1% to 7.5%, about 2% to 6%, or about 2.5% to 5% of the mass of the dental resin. In certain aspects, the amount of antibacterial monomers is about 1%, 2%, 3%, 4% or 5% of the mass of the dental resin.

When used, the amount of quaternary ammonium salts present in the protein-repellant dental resin ranges from about 1% to 30% of the mass of the dental resin, preferably about 7.5% to 15%.

When used, the amount of silver-containing nanoparticles, chlorhexidine particles, TiO2 particles or ZnO particles present in the protein-repellant dental resin ranges from about 0.01% and 20% of the mass of the dental resin, preferably about 0.08% to 10% of the mass of the dental resin.

When present, the remineralizing agent may be a single remineralizing agent or a mixture of two or more remineralizing agents. The remineralizing agents include, but are not limited to, nanoparticles of amorphous calcium phosphate (NACP).

When present, the amount of amount of NACP included in the protein-repellant dental resin ranges from about 10% to 40% of the mass of the dental resin. In particular aspects, the mass fraction of NACP in the protein-repellant dental resin is about 25%, 30% or 35% of the mass of the dental resin.

In a particular aspect, the present invention is directed to a protein-repellant dental resin comprising a dental resin, MPC, and an antibacterial agent, wherein MPC is present in an amount ranging from about 5-10% by mass of the dental resin, wherein the antibacterial agent is present in an amount ranging from about 2.5-7.5% by mass of the dental resin, and wherein the antibacterial agent is DMADDM, DMAPDM, or DMAHDM.

In a particular aspect, the present invention is directed to a protein-repellant dental resin comprising a dental resin, MPC, and an antibacterial agent, wherein MPC is present in an amount ranging from about 5-10% by mass of the dental resin, wherein the antibacterial agent is present in an amount of about 5% by mass of the dental resin, and wherein the antibacterial agent is DMADDM, DMAPDM, or DMAHDM.

In a particular aspect, the present invention is directed to a protein-repellant dental resin comprising a dental resin, MPC, and an antibacterial agent, wherein MPC is present in an amount of about 7.5% by mass of the dental resin, wherein the antibacterial agent is present in an amount ranging from about 2.5-7.5% by mass of the dental resin, and wherein the antibacterial agent is DMADDM, DMAPDM, or DMAHDM.

In a particular aspect, the present invention is directed to a protein-repellant dental resin comprising a dental resin, MPC, and an antibacterial agent, wherein MPC is present in an amount of about 7.5% by mass of the dental resin, wherein the antibacterial agent is DMAHDM and present in an amount of about 5% by mass of the dental resin.

### Protein-Repellant Dental Composite

The protein-repellant dental materials of the invention include dental composites. Thus, and in a fifth embodiment, the present invention includes a protein-repellant dental composite comprising a dental resin, a filler, and a protein-repellant agent. In certain aspects, the protein-repellant dental composite may further comprise an antibacterial agent, a remineralizing agent, or both.

The dental resin in the protein-repellant dental composite is one or more resins selected from the group consisting of bis-GMA (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer, and a heat-cured polymer. In certain aspects, the resin is a 1:1 mass ratio of bis-GMA and TEGDMA.

Alternatively, the resin used in the protein-repellant dental composites of the invention can be a protein-repellant dental resin as defined herein. When a protein-repellant dental resin of the invention is used in the protein-repellant dental composite, additional protein-repellant agent can optionally be added. Thus, the protein-repellant dental composites of the invention include protein-repellant dental composites comprising a protein-repellant dental resin and a filler, and optionally a protein-repellant agent in addition to that present in the protein-repellant dental resin.

In each aspect of this embodiment, the protein-repellant agent may be a single protein-repellant agent or a mixture of two or more protein-repellant agents. Suitable protein-repellant agents include 2-methacryloyloxyethyl phosphorylcholine (MPC), poly(hydroxyethyl methacrylate) (HEMA) and derivatives thereof, and poly(N-isopropylacrylamide) and derivatives thereof.

The amount of dental resin in the protein-repellant dental composite is between about 1% to 70% of the mass of the protein-repellant dental composite. In certain aspects, the resin is a mass fraction of from about 10% to 45%, about 20% to 40%, or about 25% to 35% of the protein-repellant dental composite. In certain other aspects, the resin is a mass fraction of about 10%, 15%, 20%, 25%, 30%, or 35% of the protein-repellant dental composite.

The filler in the protein-repellant dental composites is one or more of a glass filler, a ceramic filler, a polymer-based filler, and nanoparticles of amorphous calcium phosphate (NACP). In aspects where the filler is a glass filler, the glass filler may be barium boroaluminosilicate, strontium-alumino-fluoro-silicate glass, silicon dioxide, fluoroaluminosilicate glass, a ytterbium tri-fluoride filler, or a fiber glass filler. In certain aspects, the filler is barium boroaluminosilicate. In aspects where the filler is a ceramic filler, the ceramic filler may be a porcelain filler, a quartz filler, or a zirconia filler.

The amount of the filler in the protein-repellant dental composite is between about 5% to 90% of the mass of the protein-repellant dental composite. In certain aspects, the filler is a mass fraction of from about 10% to 85%, about 20% to 85%, or about 30% to 80% of the protein-repellant dental composite. In certain other aspects, the filler is a mass fraction of about 80%, 75%, 70%, 65%, 60%, 55%, or 50% of the protein-repellant dental composite.

When NACP is present as a filler, whether alone or in combination with one or more other fillers, it may make up between about 1% and 80% of the mass of the protein-repellant dental composite. In certain aspects, the NACP is a mass fraction of from about 5% to 60%, about 10% to 40%, about 15% to 35%, or about 20% to 30% of the protein-repellant dental composite. In certain other aspects, the NACP is a mass fraction of about 25%, 25.5%, 26%, 26.5%, 27%, 27.5%, 28%, 28.5%, 29%, 29.5%, 30%, 30.5%, 31%, 31.5%, 32%, 32.5%, 33%, or 33.5% of the protein-repellant dental composite.

The NACP particles may range in size from about 10 nm to 500 nm. In certain aspects, the NACP particles range in size from about 50 nm to 300 nm, about 50 nm to 200 nm, or about 75 nm to 200 nm.

As indicated, in some aspects the filler is two different fillers, for example, a glass filler and NACP, or a ceramic filler and NACP. In such aspects, the total amount of the two fillers in the protein-repellant dental composite will again be about 5% to 90% of the mass of the protein-repellant dental composite. As a non-limiting example, the first filler (e.g., a glass filler) may be about 50% of the mass of the composite while the second filler (e.g., NACP) is about 20% of the mass of the composite. In another example, the first filler (e.g., a glass filler) is about 40% of the mass of the composite while the second filler (e.g., NACP) is about 30% of the mass of the composite. In a further example, the first filler (e.g., a glass filler) is about 35% of the mass of the composite while the second filler (e.g., NACP) is about 35% of the mass of the composite.

The total amount of protein-repellant agent in the protein-repellant dental composite, regardless of whether the agent is added directly to the composite or whether it is encompassed in a protein-repellant dental resin used in the production of the composite, ranges from about 0.5% to 30% of the mass of the protein-repellant dental composite. In certain aspects, the range is from about 0.5% to 20%, about 0.5% to 15%, about 1% to 25%, about 1% to 20%, about 1% to 15%, about 1.5% to 20%, about 1.5% to 15%, about 1.5% to 10%, about 2% to 15%, about 2% to 10%, about 2% to 7.5%, about 2% to 5%, about 2.5% to 10%, about 2.5% to 7.5% or about 2.5% to 5% of the mass of the protein-repellant dental composite. In certain aspects, the amount of protein-repellant agent is about 0.5%, 1.0%, 1.5%, 2.0%, 2.5%, 3%, 3.5%, 4%, 4.5%, 5%, 5.5%, 6%, 6.5%, 7%, 7.5%, 8%, 8.5%, 9%, 9.5%, or 10%, of the mass of the protein-repellant dental composite.

When present, the antibacterial agent may be a single antibacterial agent or a mixture of two or more antibacterial agents. The antibacterial agents include, but are not limited to, antibacterial monomers, silver-containing nanoparticles (NAg), quaternary ammonium salts (QAS), chlorhexidine particles, TiO₂ particles and ZnO particles.

The antibacterial monomers include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). In certain aspects, the antibacterial monomer is DMADDM, DMATDM, DMATTDM, DMAPDM or DMAHDM.

When used, the amount of antibacterial monomers present in the protein-repellant dental composite ranges from about 0.1% to 15% of the mass of the protein-repellant dental composite. In certain aspects, the range is from about 0.5% to 10%, about 1% to 7.5%, about 2% to 6%, or about 2.5% to 5% of the mass of the protein-repellant dental composite. In certain aspects, the amount of antibacterial monomers is about 1%, 2%, 3%, 4% or 5% of the mass of the protein-repellant dental composite.

When used, the amount of quaternary ammonium salts present in the protein-repellant dental composite ranges from about 1% to 30% of the mass of the protein-repellant dental composite, preferably about 7.5% to about 15%.

When used, the amount of silver-containing nanoparticles, chlorhexidine particles, TiO2 particles or ZnO particles present in the protein-repellant dental composite ranges from about 0.01% and 20% of the mass of the protein-repellant dental composite, preferably about 0.08% to 10% of the mass of the protein-repellant dental composite.

When present, the remineralizing agent may be a single remineralizing agent or a mixture of two or more remineralizing agents. The remineralizing agents include, but are not limited to, nanoparticles of amorphous calcium phosphate (NACP).

When present, the amount of NACP included in the protein-repellant dental composite ranges from about 10% to 40% of the mass of the protein-repellant dental composite. In particular aspects, the mass fraction of NACP in the protein-repellant dental composite is about 25%, 30% or 35% of the mass of the protein-repellant dental composite.

In a particular aspect, the present invention is directed to a protein-repellant dental composite comprising a dental resin, a glass filler, MPC, and an antibacterial agent, wherein glass filler is present in an amount ranging from about 60-80% by mass of the dental composite, wherein MPC is present in an amount ranging from about 2-5% by mass of the dental composite, wherein the antibacterial agent is present in an amount ranging from about 1-5% by mass of the dental composite, and wherein the antibacterial agent is DMADDM, DMAPDM, or DMAHDM.

In a particular aspect, the present invention is directed to a protein-repellant dental composite comprising a dental resin, a glass filler, MPC, and an antibacterial agent, wherein glass filler is present in an amount of about 70% by mass of the dental composite, wherein MPC is present in an amount of about 3% by mass of the dental composite, wherein the antibacterial agent is present in an amount ranging from about 1-5% by mass of the dental composite, and wherein the antibacterial agent is DMADDM, DMAPDM, or DMAHDM.

In a particular aspect, the present invention is directed to a protein-repellant dental composite comprising a dental resin, a glass filler, MPC, and an antibacterial agent, wherein glass filler is present in an amount of about 70% by mass of the dental composite, wherein MPC is present in an amount ranging from about 2-5% by mass of the dental composite, wherein the antibacterial agent is present in an amount of about 1.5% by mass of the dental composite, and wherein the antibacterial agent is DMADDM, DMAPDM, or DMAHDM.

In a particular aspect, the present invention is directed to a protein-repellant dental composite comprising a dental resin, a glass filler, MPC, and an antibacterial agent, wherein glass filler is present in an amount of about 70% by mass of the dental composite, wherein MPC is present in an amount of about 3% by mass of the dental composite, wherein the antibacterial agent is DMAHDM and present in an amount of about 1.5% by mass of the dental composite.

The protein-repellant dental materials of the invention include an adhesive coating system. Thus, and in a sixth embodiment, the present invention includes a protein-repellant adhesive coating system comprising (i) a protein-repellant dental primer and (ii) a protein-repellant dental adhesive, wherein the protein-repellant dental adhesive comprises NACP. In certain aspects, the protein-repellant dental primer or the protein-repellant dental adhesive, or both may further comprise an antibacterial agent.

The protein-repellant dental primer and protein-repellant dental adhesive for use in the protein-repellant adhesive coating system are as defined herein.

The amount of protein-repellant agent in the protein-repellant dental primer ranges from about 0.5% to 50% of the mass of the protein-repellant dental primer. In certain aspects, the range is from about 1% to 25%, about 2.5% to 20%, about 4% to 15%, or about 5% to 12.5% of the mass of the protein-repellant dental primer. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10%, of the mass of the protein-repellant dental primer.

The amount of protein-repellant agent in the protein-repellant dental adhesive ranges from about 0.5% to 50% of the mass of the protein-repellant dental adhesive. In certain aspects, the range is from about 1% to 25%, about 2.5% to 20%, about 4% to 15%, or about 5% to 12.5% of the mass of the protein-repellant dental adhesive. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10%, of the mass of the protein-repellant dental adhesive.

The protein-repellant dental adhesive comprises between about 10% to 50% NACP by mass of the protein-repellant dental adhesive. In particular aspects, the mass fraction of NACP in the dental adhesive ranges from about 15% to 40% or about 20% to 35%. In certain aspects, the mass fraction of NACP in the dental adhesive is about 15%, 20%, 25%, 30%, 35% or 40%.

When present, the antibacterial agent may be a single antibacterial agent or a mixture of two or more antibacterial agents. The antibacterial agents include, but are not limited to, antibacterial monomers, silver-containing nanoparticles (NAg), quaternary ammonium salts (QAS), chlorhexidine particles, TiO₂ particles and ZnO particles.

The antibacterial monomers include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). In certain aspects, the antibacterial monomer is DMADDM, DMATDM, DMATTDM, DMAPDM or DMAHDM.

When used, the amount of antibacterial monomers present in the protein-repellant dental primer or protein-repellant dental adhesive independently ranges from about 0.5% to 50% of the mass of the protein-repellant dental primer or the protein repellant dental adhesive. In certain aspects, the range is from about 1% to 25%, about 1.5% to 20%, about 2% to 15%, or about 2.5% to 12.5% of the mass of the protein-repellant dental primer or the protein repellant dental adhesive. In certain aspects, the amount of antibacterial monomers is about 1.0%, 1.5%, 2%, 2.5%, 3%, 3.5%, 4%, 4.5%, 5%, 5.5%, 6%, 6.5%, 7%, or 7.5% of the mass of the protein-repellant dental primer or the protein repellant dental adhesive.

When used, the amount of quaternary ammonium salts present in the protein-repellant dental primer or the protein repellant dental adhesive independently ranges from about 1% to 30% of the mass of the protein-repellant dental primer or the protein repellant dental adhesive, preferably about 7.5% to 15%.

When used, the amount of silver-containing nanoparticles, chlorhexidine particles, TiO2 particles or ZnO particles present in the protein-repellant the protein-repellant dental primer or the protein-repellant dental adhesive independently ranges from about 0.01% and 20% of the mass of the protein-repellant dental primer or the protein-repellant dental adhesive, preferably about 0.08% to 10% of the mass of the protein-repellant dental primer or the protein-repellant dental adhesive.

In a particular aspect, the present invention is directed to a protein-repellant adhesive coating system comprising (i) a protein-repellant dental primer and (ii) a protein-repellant dental adhesive, wherein the protein-repellant dental primer comprises about 5-10% MPC and about 2.5-7.5% antibacterial agent by mass of the protein-repellant dental primer, wherein the antibacterial agent is DMADDM, DMAPDM, or DMAHDM, and wherein the protein-repellant dental adhesive comprises about 5-10% MPC and about 2.5-7.5% antibacterial agent by mass of the protein-repellant dental adhesive, wherein the antibacterial agent is DMADDM, DMAPDM, or DMAHDM, and wherein the protein-repellant dental adhesive further comprises about 20-40% NACP by mass of the protein-repellant dental adhesive.

In a particular aspect, the present invention is directed to a protein-repellant adhesive coating system comprising (i) a protein-repellant dental primer and (ii) a protein-repellant dental adhesive, wherein the protein-repellant dental primer comprises about 7.5% MPC and about 5% DMAHDM by mass of the protein-repellant dental primer, wherein the protein-repellant dental adhesive comprises about 7.5% MPC and about 5% DMAHDM by mass of the protein-repellant dental adhesive, and wherein the protein-repellant dental adhesive further comprises about 30% NACP by mass of the protein-repellant dental adhesive.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF THE DRAWINGS

FIG. 1. Dentin bond strength results (mean±sd; n=10). Dissimilar letters indicate values that are significantly different from each other (p<0.05). Bonding agent containing 7.5% MPC had a bond strength similar to that of control without MPC (p>0.1).

FIG. 2. The amount of bovine serum albumin (BSA) protein adsorption onto resin surfaces (mean±sd; n=6). Incorporation of MPC into primer and adhesive significantly decreased the amount of protein adsorption on resin surfaces. However, the amount of protein adsorption increased at MPC mass fractions ≧11.25%. Dissimilar letters indicate values that are significantly different from each other (p<0.05).

FIGS. 3A-3B. Representative live/dead staining images of dental plaque microcosm biofilms grown for 2 days on resin disks: FIG. 3A—SBMP control; FIG. 3B—SBMP+7.5% MPC. In FIG. 3A, biofilms on control disks had primarily live bacteria covering the entire disk. In FIG. 3B, substantial decreases in bacterial adhesion occurred when MPC was incorporated into primer and adhesive. The live bacteria were stained green, and the dead bacteria were stained red.

FIGS. 4A-4D. Colony-forming unit (CFU) counts and metabolic activity of dental plaque microcosm biofilms grown for 2 days on resin disks (mean±sd; n=6). FIG. 4A—Total microorganism CFU; FIG. 4B—total streptococci CFU; FIG. 4C—mutans streptococci CFU; FIG. 4D—metabolic activity of biofilms. All three CFU counts on the SBMP with 7.5% MPC were an order of magnitude less than that on SBMP control (p<0.05). Biofilms on SBMP with 7.5% MPC had metabolic activity that was about 40% that of control (p<0.05).

FIG. 5. Dentin bond strength results using extracted human molars (mean±sd; n=10). Bars with dissimilar letters indicate values that are significantly different from each other (p<0.05). The first six groups had shear bond strengths that were not significantly different from each other (p>0.1). The last two groups had shear bond strengths that were significantly lower than SBMP control (p<0.05).

FIG. 6. Protein adsorption onto resin surfaces (mean±sd; n=6). The resin with 7.5% MPC+5% DMAHDM had protein adsorption that was about 20-fold less than that of SBMP control (p<0.05). Bars with dissimilar letters indicate values that are significantly different from each other (p<0.05).

FIGS. 7A-7D. Representative live/dead staining images of biofilms adherent on resin disks. The live bacteria were stained green, and the dead bacteria were stained red. SBMP control resin (FIG. 7A) was covered with biofilms consisting of mostly live bacteria while those on the 5% DMAHDM resin (FIG. 7C) mainly consisted of dead bacteria. The resin with 7.5% MPC (FIG. 7B) had much less bacterial adhesion. The resin containing 7.5% MPC+5% DMAHDM (FIG. 7D) had much less bacterial adhesion, and the biofilms consisted of primarily dead bacteria.

FIGS. 8A-8C. Colony-forming unit (CFU) counts of 2-day biofilms on resin disks for: FIG. 8A—total microorganisms; FIG. 8B—total streptococci; FIG. 8C—mutans streptococci (mean sd; n=6). Using MPC and DMAHDM together in the same adhesive resin resulted in much lower biofilm CFU than using MPC or DMAHDM alone. All three CFU counts on the resin with 7.5% MPC+5% DMAHDM were more than 4 orders of magnitude lower than those on SBMP control (p<0.05).

FIGS. 9A-9B. Biofilm viability on resin disks. FIG. 9A—Metabolic activity; FIG. 9B—lactic acid production of 2-day biofilms (mean±sd; n=6). Metabolic activity and lactic acid production of biofilms on resin containing 7.5% MPC+5% DMAHDM were nearly 25-fold less than that on SBMP control (p<0.05).

FIGS. 10A-10B. Mechanical properties of dental composites. FIG. 10A—Flexural strength; FIG. 10B—elastic modulus (mean±s.d.; n=6). The composite specimens were immersed in distilled water at 37° C. for 24 h, and then fractured while being wet, within a few minutes after being taken out of the water. Dissimilar letters indicate values that are significantly different from each other (P<0.05).

FIG. 11. Protein adsorption onto composite surfaces (mean±s.d.; n=6). The composite with 3% MPC had the lowest amount of protein adsorption, which was about 1/12 those of commercial composite control and experimental composite with 0% MPC (P<0.05). Dissimilar letters indicate values that are significantly different from each other (P<0.05).

FIGS. 12A-12E. Oral microcosm bacteria early-attachment on composites at 4 h. FIGS. 12A-12D—representative live/dead staining images of bacteria on composite surfaces; FIG. 12E—area fraction of green staining of live bacteria coverage on composite surface (mean±s.d.; n=6). Composite control had much more bacteria attachment. Increasing the MPC content decreased the bacterial attachment. Dissimilar letters in FIG. 12E indicate values that are significantly different from each other (P<0.05).

FIGS. 13A-13E. Oral microcosm biofilm growth on composites at 48 h. FIGS. 13A-13D—representative live/dead staining images of biofilms on composite surfaces; FIG. 13E—area fraction of green staining of live bacteria coverage on composite surface (mean±s.d.; n=6). Increasing the MPC mass fraction decreased the biofilm coverage on the composite. The composite with 3% MPC had the least biofilm coverage (P<0.05). In FIG. 13E, dissimilar letters indicate values that are significantly different from each other (P<0.05).

FIGS. 14A-14C. Colony-forming unit (CFU) counts of 2-day biofilms on composites. FIG. 14A—Total microorganisms; FIG. 14B—total streptococci; FIG. 14C—mutans streptococci (mean±s.d.; n=6). Increasing the MPC mass fraction decreased the biofilm CFU on the composite (P<0.05). All three CFU counts on the composite with 3% MPC were much lower than that of commercial composite control and the experimental composite with 0% MPC (P<0.05). In each plot, dissimilar letters indicate values that are significantly different from each other (P<0.05).

FIGS. 15A-15B. Mechanical properties of composites: FIG. 15A—Flexural strength; FIG. 15B—elastic modulus (mean±sd; n=6). The composite with 3% MPC+1.5% DMAHDM had strength and elastic modulus similar to those of a commercial control (p>0.1). Bars with dissimilar letters indicate values that are significantly different from each other (p<0.05).

FIG. 16. Protein adsorption onto composite surfaces (mean±sd; n=6). The composite with 3% MPC, and the composite with 3% MPC+1.5% DMAHDM, both had much less protein adsorption, which was about 1/10 that of commercial control composite (p<0.05). Bars with dissimilar letters indicate values that are significantly different from each other (p<0.05).

FIGS. 17A-17F. Representative live/dead staining images of biofilms adherent on composite disks cultured for 2 days: FIG. 17A—Commercial control composite; FIG. 17B—control composite with 0% MPC+0% DMAHDM; FIG. 17C—composite with 3% MPC; FIG. 17D—composite with 1.5% DMAHDM; FIG. 17E—composite with 3% MPC+1.5% DMAHDM. FIG. 17F provides area fraction of green staining of live bacteria coverage on composite surface (mean±sd; n=6). The live bacteria were stained green, and the dead bacteria were stained red. When live and dead bacteria were in close proximity or on the top of each other, the staining had yellow or orange colors. The composite with 3% MPC+1.5% DMAHDM had greatly decreased bacterial adhesion, and the biofilms consisted of primarily dead bacteria. Dissimilar letters in FIG. 17E indicate values that are significantly different from each other (p<0.05).

FIGS. 18A-18B. Biofilm viability on composite disks cultured for 2 days: FIG. 18A—metabolic activity; FIG. 18B—lactic acid production (mean±sd; n=6). Biofilms on the composite with 3% MPC+1.5% DMAHDM had metabolic activity that was about 5% that on commercial control (p<0.05). Lactic acid production by the biofilms on the composite with 3% MPC+1.5% DMAHDM was about 7% that on the commercial control (p<0.05). In each plot, values with dissimilar letters are significantly different from each other (p<0.05).

FIGS. 19A-19C. Biofilm CFU counts on composite disks cultured for 2 days: FIG. 19A—total microorganisms; FIG. 19B—total streptococci; FIG. 19C—mutans streptococci (mean sd; n=6). All three CFU counts on the composite with 3% MPC+1.5% DMAHDM were more than 3 orders of magnitude lower than those on commercial control. In each plot, values with dissimilar letters are significantly different from each other (p<0.05).

FIG. 20. Dentin shear bond strengths (mean±sd; n=10). All the strengths were not significantly different from each other, except the 7.5% MPC+5% DMAHDM+40% NACP group. Bars with dissimilar letters indicate values that are significantly different (p<0.05).

FIGS. 21A-21E. Typical SEM images showing the thickness of coating layers. FIG. 21A—SBMP control; FIG. 21B—7.5% MPC+5% DMAHDM; FIG. 21C—7.5% MPC+5% DMAHDM+20% NACP; FIG. 21D—7.5% MPC+5% DMAHDM+30% NACP. “D”: dentin, “C”: composite, and “A”: adhesive. The mean value of the coating thickness for each adhesive are shown in FIG. 21E (mean±sd; n=6). Bars with dissimilar letters indicate values that are significantly different (p<0.05).

FIGS. 22A-22D. Representative SEM images of dentin-adhesive interfaces and surface texture of coating layers. FIG. 22A—7.5% MPC+5% DMAHDM+30% NACP; FIG. 22B—7.5% MPC+5% DMAHDM+30% NACP at a higher magnification; FIG. 22C—SBMP control; FIG. 22D—7.5% MPC+5% DMAHDM+30% NACP. In FIG. 22A, adhesives filled the dentinal tubules and formed resin tags “T”. “HL” indicates the hybrid layer between the adhesive and the underlying mineralized dentin. Arrows in FIG. 22B indicate NACP in the dentinal tubules. Arrows indicate the bubbles were observed in some area of FIG. 22C. In contrast, FIG. 22D showed uniform coating surfaces which could seal all the dentinal surfaces.

FIG. 23. The amount of protein adsorption of each group (mean±sd; n=6). The SBMP control had the highest amount of protein adsorption that was nearly 18-fold higher than the other groups (p<0.05).

FIGS. 24A-24D. Representative live/dead staining images of biofilms adherent on the disk. The live bacteria were stained green, and the dead bacteria were stained red. However, when live/dead bacteria were in close proximity or on the top of each other, the staining had yellow/orange colors. The SBMP control was fully covered by primarily live bacteria (FIG. 24A). In contrast, FIG. 24B (7.5% MPC+5% DMAHDM), FIG. 24C (7.5% MPC+5% DMAHDM+20% NACP) and FIG. 24D (7.5% MPC+5% DMAHDM+30% NACP) showed noticeably decreased in bacterial adhesion, and the biofilms consisted of primarily dead bacteria.

FIGS. 25A-25B. Biofilm viability on resin composite disks: FIG. 25A—metabolic activity; FIG. 25B—lactic acid production (mean±sd; n=6). Biofilms on MPC-DMAHDM-NACP containing adhesive had metabolic activity that was about 20-fold less than those on SBMP control and the lactic acid production was about 25-fold less than those on SBMP control.

FIGS. 26A-26C. Colony-forming unit (CFU) counts for: FIG. 26A—total microorganisms; FIG. 26B—total streptococci; FIG. 26C—mutans streptococci (mean±sd; n=6). All three CFU counts on the MPC-DMAHDM-NACP containing adhesive were more than 4 orders of magnitude lower than that of SBMP control (p<0.05).

### DETAILED DESCRIPTION

Described herein are protein-repellent dental materials that can be used in a wide variety of dental applications. The dental materials have the unique ability to repel attachment of proteins to the surface of the materials due to the inclusion of a protein-repellent agent in the material. The inclusion of the protein-repellant agent serves to inhibit protein attachment without altering the strength of the material or other important characteristics of the material.

The protein-repellent dental materials of the invention include dental primers, dental adhesives, dental resins, dental composites, dental bonding systems and the like, as well as dental cements, dental sealants, dental bases and dental liners, each of which is protein-repellent and each of which comprises a protein-repellent agent.

In each embodiment and aspect of the invention, the protein-repellant agent may be a single protein-repellant agent or a mixture of two or more protein-repellant agents. Suitable protein-repellent agents include those that either contain or have the potential to contain methacrylate/acrylate functionality. This includes protein-repellent agents that are based on poly(ethylene glycol) (PEG). There are many polyethylene glycol-based methacrylates and derivatives that impart protein-repellent characteristics. Other protein-repellent agents include zwitterionic polymers. Zwitterionic polymers contain alternating positive and negative charges in close proximity. This allows these materials to interact with water through strong electrostatic attractions. This bound water is difficult to displace by proteins and microorganisms, resulting in protein-repellent properties. Other protein-repellent agents include compounds such as poly(hydroxyethyl methacrylate) (HEMA) and its derivatives and poly(N-isopropylacrylamide) and its derivatives. An exemplary protein-repellant agent is 2-methacryloyloxyethyl phosphorylcholine (MPC)

The protein-repellent technology can be combined with antibacterial and remineralization agents, methods and compositions (for example, but not limited to, the antibacterial and remineralization agents, methods and compositions described in WO2012/003290, WO2013/119901, and WO2014/062347) for use in the dental primers, adhesives, resins, composites, bonding systems, cements, sealants, bases and liners mentioned above.

As provided in the Summary section above, the invention includes dental primers, dental adhesives, dental resins, dental composites that encompass a protein repellant agent, along with the use of these dental materials in such applications as the dental bonding systems described and defined herein.

The dental primers of the invention comprise any primer, or combination of primers, that is suitable for dental use in a subject, such as a human. Suitable primers will be those commonly used in dental applications. Exemplary primers comprise Bisphenol A diglycidyl methacrylate (Bis-GMA), glycerol dimethacrylate (GDMA), 2-hydroxyethyl methacrylate (HEMA), mono-2-methacryloyloxyethyl phthalate (MMEP), methacrylic acid (MA), methyl methacrylate (MMA), 4-acryloyloxyethyl trimellitate anhydride (4-AETA), N-phenylglycine glycidyl methacrylate (NPG-GMA), N-tolylglycine glycidyl methacrylate or N-(2-hydroxy-3-((2-methyl-1-oxo-2-propenyl)oxy)propyl)-N-tolyl glycine (NTG-GMA), pyromellitic diethylmethacrylate or 2,5-dimethacryloyloxyethyloxycarbonyl-1,4-benzenedicarboxylic acid (PMDM), pyromellitic glycerol dimethacrylate or 2,5-bis(1,3-dimethacryloyloxyprop-2-yloxycarbonyl)benzene-1,4-dicarboxylic acid (PMGDM), and triethylene glycol dimethacrylate (TEGDMA). In one example, a suitable primer comprises SBMP primer comprising 35-45% 2-hydroxyethylmethacrylate (HEMA), 10-20% copolymer of acrylic and itaconic acids, 40-50% water. In another example, the primer comprises PMGDM/HEMA at 3.3/1 ratio+1% BAPO+50% acetone.

Similarly, the dental adhesives of the invention comprise any adhesive, or combination of adhesives, that is suitable for dental use in a subject, such as a human. Suitable adhesives will be those commonly used in dental applications. Exemplary adhesives comprise ethoxylated bisphenol A glycol dimethacrylate (Bis-EMA), bisphenol A diglycidyl methacrylate (Bis-GMA), 2-hydroxyethyl methacrylate (HEMA), triethylene glycol dimethacrylate (TEGDMA), urethane dimethacrylate (UDMA), 4-methacryloyloxyethyl trimellitate anhydride (4-META), methacrylic acid (MA), methyl methacrylate (MMA), 4-acryloyloxyethyl trimellitate anhydride (4-AETA), ethyleneglycol dimethacrylate (EGDMA), glycerol dimethacrylate (GDMA), glycerol phosphate dimethacrylate (GPDM), pyromellitic glycerol dimethacrylate or 2,5-bis(1,3-dimethacryloyloxyprop-2-yloxycarbonyl)benzene-1,4-dicarboxylic acid (PMGDM). In one example, a suitable adhesive comprises SBMP adhesive comprising 60-70% BisGMA and 30-40% HEM. In another example, the adhesive comprises BisGMA/TEGMA at 7/3 ratio+1% BAPO.

### Protein-Repellant Dental Primer and Protein-Repellant Dental Adhesive

The invention includes protein-repellant dental primers comprising a dental primer, as defined above, and a protein-repellant agent. The amount of protein-repellant agent in the protein-repellant dental primer ranges from about 0.5% to 50% of the mass of the dental primer. In certain aspects, the range is from about 1% to 25%, about 2.5% to 20%, about 4% to 15%, or about 5% to 12.5% of the mass of the dental primer. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10% of the mass of the dental primer

The invention also includes protein-repellant dental adhesives comprising a dental adhesive, as defined above, and a protein-repellant agent. The amount of protein-repellant agent in the protein-repellant dental adhesive ranges from about 0.5% to 50% of the mass of the dental adhesive. In certain aspects, the range is from about 1% to 25%, about 2.5% to 20%, about 4% to 15%, or about 5% to 12.5% of the mass of the dental adhesive. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10% of the mass of the dental adhesive.

The protein-repellant agent that is encompassed in the protein-repellant dental primers and protein-repellant dental adhesives of the invention is 2-methacryloyloxyethyl phosphorylcholine (MPC).

In certain aspects, protein-repellant dental primers and protein-repellant dental adhesives of the invention may further comprise an antibacterial agent, a remineralizing agent, or both.

The antibacterial agents include, but are not limited to, antibacterial monomers, silver-containing nanoparticles (NAg), quaternary ammonium salts (QAS), chlorhexidine particles, TiO2 particles and ZnO particles.

The antibacterial monomers differ based on the length of the alkyl chain and include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM).

The antibacterial monomers can be incorporated into the protein-repellant dental primers and protein-repellant dental adhesives at antibacterial monomer mass fractions ranging from about 0.5% to 50% of dental primer or dental adhesive, preferably from about 2% to 20% of the mass of the dental primer or dental adhesive. In certain aspects, the antibacterial monomers can be incorporated into the dental primer or dental adhesive at antibacterial monomer mass fractions ranging from about 1% to 50%, from about 1% to 25%, from about 1.5% to 20%, from about 2% to 20%, from about 2% to 15%, from about 2.5% to 25%, from about 2.5% to 20%, from about 2.5% to 15%, from about 2.5% to 12.5%, from about 5% to 25%, from about 5% to 20%, from about 5% to 15%, from about 5% to 10%, from about 7.5% to 25%, from about 7.5% to 20%, from about 7.5% to 15% or from about 7.5% to 12.5% of the mass of the dental primer or dental adhesive. In particular aspects, the amount of the antibacterial monomers present in the dental primer or dental adhesive is a combined amount of about 0.5, 1, 2, 2.5, 3, 4, 5, 6, 7, 7.5, 8, 9, 10, 11, 12, 12.5, 13, 14, 15, 16, 17, 17.5, 18, 19, 20, 21, 22, 22.5, 23, 24, 25, 26, 27, 27.5, 28, 29, 30, 31, 32, 33, 34, 35, 36, 37, 38, 39, 40, 41, 42, 43, 44, 45, 46, 47, 48, 49 or 50% of the mass of the dental primer or dental adhesive. In certain aspects of the invention, the amount of the antibacterial monomers incorporated into the dental primer or dental adhesive ranges from about 1% to 50% of the mass of the dental primer or dental adhesive. In particular aspects, the amount of the antibacterial monomers present in the dental primer or dental adhesive is about 10%, 7.5%, or 5% of the mass of the dental primer or dental adhesive. In certain aspects, one of the antibacterial monomers identified herein is included in the dental primer or dental adhesive. In certain other aspects, two, three, four, or more of the antibacterial monomers identified herein are included in the dental primer or dental adhesive.

Suitable silver-containing nanoparticles (NAg) include, but are not limited to, silver 2-ethylhexanoate salt, silver-containing glass particles and silver benzoate. In addition to silver salts, pre-formed silver nanoparticles can be used. When present, NAg may make up between about 0.001% and 20% of a mass fraction of the material (i.e., the dental primer or dental adhesive). In certain aspects, NAg will make up between about 0.001% and 3%, about 0.05% and 1%, about 0.05% and 2%, about 0.05% and 5%, about 0.08% and 10%, or about 0.1% and 0.5%, of a mass fraction of the material, or about 0.01%, 0.08%, 0.15%, 0.20%, 0.25%, 0.30%, 0.35%, 0.40%, 0.45%, 0.50%, 1.0%, 1.5%, 2.0%, 2.5%, 3.0%, 3.5%, 4.0%, 4.5% or 5.0% of a mass fraction of the material. In one aspect, NAg makes up about 0.1% of a mass fraction of the material. The silver particle size can range from about 1 nm to 1000 nm, and in one aspect, from about 2 nm to 500 nm. In certain aspects, the amount of NAg present in the material ranges from about 0.05% and 5% of the mass of the material. In particular aspects, the amount of NAg present in the material is a mass fraction of about 0.1%, 0.25%, or 0.5%.

Suitable quaternary ammonium salts (QASs) include both polymerizable monomers and non-polymerizable small molecules, and include, but are not limited to, bis(2-methacryloyloxy-ethyl) dimethyl-ammonium bromide (QADM), methacryloyloxydodecylpyridinium bromide, methacryloxylethyl benzyl dimethyl ammonium chloride, methacryloxylethyl m-chloro benzyl dimethyl ammonium chloride, methacryloxylethyl cetyl dimethyl ammonium chloride, cetylpyridinium chloride, and methacryloxylethyl cetyl ammonium chloride, QAS chlorides, QAS bromides, QAS monomethacrylates, QAS dimethacrylates, and pre-fabricated QAS particles. When present, the QAS may make up between about 1% and 50% of a mass fraction of the material (i.e., the dental primer or dental adhesive). In certain aspects, the QAS will make up between about 2% and 25%, about 3% and 15%, about 5% and 20%, or about 7.5% and 15% of a mass fraction of the material, or about 1%, 2.5%, 5%, 7.5%, 10%, 12.5, 15%, 17.5%, 20%, 22.5%, 25%, 27.5% or 30% of a mass fraction of the material.

In embodiments where the material (i.e., dental primer or dental adhesive) comprises a remineralizing agent, such agents include, but are not limited to, nanoparticles of amorphous calcium phosphate (NACP). NACP comprises nanometer-sized amorphous calcium phosphate (Ca₃[PO₄]₂) particles that can be used to produce a material with high Ca and PO₄ release, improved mechanical properties, and improved antibacterial properties. The materials that include NACP exhibit greatly increased ion release at acidic, cariogenic pH, when these ions are most needed to inhibit caries.

The NACP included in the materials of the present invention, when present, will vary in size, but at least about 50, 55, 60, 65, 70, 75, 80, 85, 86, 87, 88, 89, 90, 91, 92, 93, 94, 95, 96, 97, 98 or 99% of the particles, or all of the particles (100%), have an average diameter of between about 10 nm and 500 nm. In certain aspects, the average diameter will be between about 25 nm and 400 nm, about 50 nm and 300 nm, about 75 nm and 200 nm, or about 100 nm and 150 nm. In a particular aspect, the NACP particles have an average diameter of between about 50 nm and 200 nm.

The amount of NACP included in the materials may vary, but the NACP will generally comprise about 1% to 90% of the mass of the material. In certain aspects, the NACP will comprise about 1% to 40%, about 5% to 70%, about 5% to 60%, about 5% to 50%, about 5% to 45%, about 5% to 40%, about 5% to 30%, about 10% to 90%, about 10% to 80%, about 10% to 70%, about 10% to 60%, about 10% to 50%, about 10% to 40%, about 15% to 70%, about 15% to 60%, about 15% to 50%, about 15% to 40%, about 20% to 90%, about 85% to 70%, about 20% to 80%, about 20% to 70%, about 20% to 60%, about 20% to 50%, about 20% to 40%, about 25% to 70%, about 25% to 60%, about 25% to 50%, about 25% to 40%, about 30% to 90%, about 30% to 85%, about 30% to 80%, about 30% to 75%, about 30% to 70%, about 30% to 60%, about 15% to 45%, about 15% to 35%, or about 15% to 25% of the mass of the material. In certain other aspects, the NACP is a mass fraction of about 25%, 25.5%, 26%, 26.5%, 27%, 27.5%, 28%, 28.5%, 29%, 29.5% or 30% of the material. In particular aspects, the NACP will comprise about 5, 6, 7, 8, 9, 10, 11, 12, 13, 14, 15, 16, 17, 18, 19, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90% of the mass of the material. In certain aspects, the NACP will range from about 10% to 40% of the mass of the material.

### Protein-Repellant Dental Resin

The invention also includes protein-repellant dental resins comprising a dental resin and a protein-repellant agent.

The dental resin may be any resin (or combination of resins) that is suitable for dental use in a subject, such as a human. Such resins typically comprise a matrix that is of a hardenable dental polymer. Exemplary resins include bis-GMA (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer and a heat-cured polymer, as well combinations of two or more of these polymers. A suitable combination is Bis-GMA and TEGDMA at 1:1 mass ratio.

The dental resins may be rendered light-curable through the addition of appropriate compounds to the resin. For example, camphorquinone and ethyl 4-N,N-dimethylaminobenzoate may be added to a resin comprising Bis-GMA and TEGDMA thereby rendering the resin light-curable. In a particular embodiment, about 0.2% camphorquinone and about 0.8% ethyl 4-N,N-dimethylaminobenzoate may be added to a resin comprising Bis-GMA and TEGDMA in about a 1:1 mass ratio to render the resin light-curable.

The amount of protein-repellant agent in the protein-repellant dental resin ranges from about 0.5% to 30% of the mass of the dental resin. In certain aspects, the range is from about 1% to 25%, about 1.5% to 20%, about 2% to 15%, or about 2.5% to 10% of the mass of the dental resin. In certain aspects, the amount of protein-repellant agent is about 0.5%, 1.0%, 1.5%, 2.0%, 2.5%, 3%, 3.5%, 4%, 4.5%, 5%, 5.5%, 6%, 6.5%, 7%, 7.5%, 8%, 8.5%, 9%, 9.5%, or 10%, of the mass of the dental resin.

In certain aspects, the protein-repellant dental resins of the invention may further comprise an antibacterial agent, a remineralizing agent, or both.

The antibacterial agents include, but are not limited to, antibacterial monomers, silver-containing nanoparticles (NAg), quaternary ammonium salts (QAS), chlorhexidine particles, TiO2 particles and ZnO particles.

The antibacterial monomers differ based on the length of the alkyl chain and include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM).

One or more antibacterial monomers may be present in the protein-repellant dental resins of the invention in an amount ranging from about 0.5% to 15% by mass of the dental resin. In certain aspects, the protein-repellant dental resin comprises an amount of from about 0.5% to 12.5%, about 0.5% to 10%, about 1% to 12.5%, about 1% to 10%, about 1% to 7.5%, about 2% to 10%, about 2% to 7.5%, about 2% to 6%, about 2.5% to 7.5%, about 2.5% to 6%, about 2.5% to 5%, or about 2.5% to 4% of the mass of the dental resin. In particular aspects, the amount of the one or more antibacterial monomers present in the protein-repellant dental resin is a combined amount of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15% of the mass of the dental resin.

When present, suitable QASs include both polymerizable monomers and non-polymerizable small molecules, and include, but are not limited to, bis(2-methacryloyloxy-ethyl) dimethyl-ammonium bromide (QADM), methacryloyloxydodecylpyridinium bromide, methacryloxylethyl benzyl dimethyl ammonium chloride, methacryloxylethyl m-chloro benzyl dimethyl ammonium chloride, methacryloxylethyl cetyl dimethyl ammonium chloride, cetylpyridinium chloride, and methacryloxylethyl cetyl ammonium chloride, QAS chlorides, QAS bromides, QAS monomethacrylates, QAS dimethacrylates, and pre-fabricated QAS particles. The QAS may make up between about 1% and 30% by mass of the dental resin. In certain aspects, the QAS will make up between about 2% and 25%, about 5% and 20%, or about 7.5% and 15% by mass of the dental resin, or about 1%, 2.5%, 5%, 7.5%, 10%, 12.5, 15%, 17.5%, 20%, 22.5%, 25%, 27.5% or 30% by mass of the dental resin.

When present, suitable silver-containing nanoparticles (NAg) include, but are not limited to, silver 2-ethylhexanoate salt, silver-containing glass particles and silver benzoate. In addition to silver salts, pre-formed silver nanoparticles can be used. NAg may make up between about 0.01% and 20% by mass of the dental resin. In certain aspects, NAg will make up between about 0.05% and 5%, or about 0.08% and 10% by mass of the dental resin, or about 0.01%, 0.08%, 0.15%, 0.20%, 0.25%, 0.30%, 0.35%, 0.40%, 0.45%, 0.50%, 1.0%, 1.5%, 2.0%, 2.5%, 3.0%, 3.5%, 4.0%, 4.5% or 5.0% by mass of the dental resin. In one aspect, NAg makes up about 0.08% by mass of the dental resin. The silver particle size can range from about 1 nm to 1000 nm, and in one aspect, from about 2 nm to 500 nm.

In embodiments where the protein-repellant dental rein comprises a remineralizing agent, such agents include, but are not limited to, nanoparticles of amorphous calcium phosphate (NACP). NACP comprises nanometer-sized amorphous calcium phosphate (Ca₃[PO₄]₂) particles that can be used to produce a material with high Ca and PO₄ release, improved mechanical properties, and improved antibacterial properties. The materials that include NACP exhibit greatly increased ion release at acidic, cariogenic pH, when these ions are most needed to inhibit caries.

The NACP included in the protein-repellant dental reins of the present invention, when present, will vary in size, but at least about 50, 55, 60, 65, 70, 75, 80, 85, 86, 87, 88, 89, 90, 91, 92, 93, 94, 95, 96, 97, 98 or 99% of the particles, or all of the particles (100%), have an average diameter of between about 10 nm and 500 nm. In certain aspects, the average diameter will be between about 25 nm and 400 nm, about 50 nm and 300 nm, about 75 nm and 200 nm, or about 100 nm and 150 nm. In a particular aspect, the NACP particles have an average diameter of between about 50 nm and 200 nm.

The amount of NACP included in the protein-repellant dental reins may vary, but the NACP will generally comprise about 1% to 90% by mass of the dental resin. In certain aspects, the NACP will comprise about 1% to 40%, about 5% to 70%, about 5% to 60%, about 5% to 50%, about 5% to 45%, about 5% to 40%, about 5% to 30%, about 10% to 90%, about 10% to 80%, about 10% to 70%, about 10% to 60%, about 10% to 50%, about 10% to 40%, about 15% to 70%, about 15% to 60%, about 15% to 50%, about 15% to 40%, about 20% to 90%, about 85% to 70%, about 20% to 80%, about 20% to 70%, about 20% to 60%, about 20% to 50%, about 20% to 40%, about 25% to 70%, about 25% to 60%, about 25% to 50%, about 25% to 40%, about 30% to 90%, about 30% to 85%, about 30% to 80%, about 30% to 75%, about 30% to 70%, about 30% to 60%, about 15% to 45%, about 15% to 35%, or about 15% to 25% by mass of the dental resin. In certain other aspects, the NACP is a mass fraction of about 25%, 25.5%, 26%, 26.5%, 27%, 27.5%, 28%, 28.5%, 29%, 29.5% or 30% of the resin. In particular aspects, the NACP will comprise about 5, 6, 7, 8, 9, 10, 11, 12, 13, 14, 15, 16, 17, 18, 19, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90% of the mass of the resin. In certain aspects, the NACP will range from about 10% to 40% of the mass of the resin.

### Protein-Repellant Dental Composite

The invention also includes protein-repellant dental composites comprising a dental resin, a filler, and a protein-repellant agent. In certain aspects, the protein-repellant dental composite may further comprise an antibacterial agent, a remineralizing agent, or both.

The protein-repellant dental composites can be prepared by combining a protein-repellant dental resin, as defined herein, and a filler. Alternatively, the protein-repellant dental composites can be prepared by combining a dental resin, as defined herein, a filler, and a separate a protein-repellant agent.

The dental resins used in the protein-repellant dental composites may be any of the dental resins described herein, or a combination of the dental resins of the present invention. Similarly, the protein-repellant dental resins used in the protein-repellant dental composites may be any of the protein-repellant dental resins described herein, or a combination of the protein-repellant dental resins of the present invention. The dental resins and protein-repellant dental resins may independently comprise an antibacterial agent, a remineralizing agent, or both, as defined above.

The amount of dental resin or protein-repellant dental resin present in the protein-repellant dental composites of the present invention may vary, but the resin will generally comprise about 1% to 70% of the mass of the protein-repellant dental composite. In certain aspects, the resin will comprise about 1% to 45%, about 1% to 40%, about 1% to 35%, about 1% to 30%, about 1% to 25%, about 1% to 20%, about 1% to 15%, about 2.5% to 45%, about 2.5% to 40%, about 2.5% to 35%, about 2.5% to 30%, about 2.5% to 25%, about 2.5% to 20%, about 2.5% to 15%, about 5% to 45%, about 5% to 40%, about 5% to 35%, about 5% to 30%, about 5% to 25%, about 5% to 20%, about 5% to 15%, about 7.5% to 45%, about 7.5% to 40%, about 7.5% to 35%, about 7.5% to 30%, about 7.5% to 25%, about 7.5% to 20%, about 7.5% to 15%, about 10% to 45%, about 10% to 40%, about 10% to 35%, about 10% to 30%, about 10% to 25%, about 10% to 20%, about 10% to 15%, about 15% to 45%, about 15% to 40%, about 15% to 35%, about 15% to 30%, about 15% to 25%, about 15% to 20%, about 20% to 45%, about 20% to 40%, about 20% to 35%, about 20% to 30%, about 20% to 25%, about 25% to 45%, about 25% to 40%, about 25% to 35%, or about 25% to 30% of the mass of the protein-repellant dental composite. In certain other aspects, the resin is a mass fraction of from about 10%, 15%, 20%, 25%, 30%, or 35% of the protein-repellant dental composite. In particular aspects, the resin will comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70% of the mass of the protein-repellant dental composite.

The filler is used to increase the strength of the protein-repellant dental composite. Suitable fillers include glass fillers, ceramic fillers, polymer-based fillers and NACP, or any combination thereof. Particular examples of suitable glass fillers include barium boroaluminosilicate, strontium-alumino-fluoro-silicate glass, silicon dioxide, fluoroaluminosilicate glass, a ytterbium tri-fluoride filler, and a fiber glass filler. Particular examples of suitable ceramic fillers include any dental ceramic such as a porcelain filler, a quartz filler, and a zirconia filler. Polymer-based filler includes dental polymer that is pre-polymerized and then ground into filler particles, and polymer fibers. NACP comprises nanometer-sized amorphous calcium phosphate (Ca₃[PO₄]₂) particles that can be used to produce photo-cured nanocomposites with high Ca and PO₄ release, improved mechanical properties, and improved antibacterial properties. The protein-repellant dental composites that include NACP exhibit greatly increased ion release at acidic, cariogenic pH, when these ions are most needed to inhibit caries.

The amount of filler present in the protein-repellant dental composite may vary, but the filler will generally comprise about 5% to 90% of the mass of the protein-repellant dental composite. In certain aspects, the filler will comprise about 10% to 85%, about 10% to 80%, about 10% to 70%, about 10% to 60%, about 10% to 50%, about 10% to 40%, about 20% to 80%, about 20% to 70%, about 20% to 60%, about 20% to 50%, about 30% to 80%, about 30% to 70%, about 30% to 60%, about 40% to 80%, about 40% to 70%, about 40% to 60%, about 50% to 80%, about 50% to 70%, or about 45% to 55% of the mass of the composite. In certain other aspects, the filler is a mass fraction of about 80%, 75%, 70%, 65%, 60%, 55%, or 50% of the composite. In particular aspects, the filler comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84 or 85% of the mass of the protein-repellant dental composite.

When present, the amount of NACP included as a filler may vary, but the NACP will generally comprise about 1% to 80% of the mass of the protein-repellant dental composite. In certain aspects, the NACP will comprise about 5% to 70%, about 5% to 60%, about 5% to 50%, about 5% to 40%, about 10% to 90%, about 10% to 80%, about 10% to 70%, about 10% to 60%, about 10% to 50%, about 10% to 40%, about 15% to 70%, about 15% to 60%, about 15% to 50%, about 15% to 40%, about 20% to 90%, about 85% to 70%, about 20% to 80%, about 20% to 70%, about 20% to 60%, about 20% to 50%, about 20% to 40%, about 25% to 70%, about 25% to 60%, about 25% to 50%, about 25% to 40%, about 30% to 90%, about 30% to 85%, about 30% to 80%, about 30% to 75%, about 30% to 70%, about 30% to 60%, about 15% to 45%, about 15% to 35%, or about 15% to 25% of the mass of the composite. In certain other aspects, the NACP is a mass fraction of about 25%, 25.5%, 26%, 26.5%, 27%, 27.5%, 28%, 28.5%, 29%, 29.5%, 30%, 30.5%, 31%, 31.5%, 32%, 32.5%, 33%, or 33.5% of the composite. In particular aspects, the NACP will comprise about 5, 6, 7, 8, 9, 10, 11, 12, 13, 14, 15, 16, 17, 18, 19, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90% of the mass of the composite.

The size of the particles of the filler will depend on the identity of the filler or fillers. As an example, in an embodiment where barium boroaluminosilicate glass particles serve as the filler or one of fillers, the median particle diameter may be between about 0.1 and 10 μm, or between about 1.0 μm and 5 μm. Thus, the median particle diameter of the fillers used in the protein-repellant dental composites of the present invention may, in one aspect where barium boroaluminosilicate glass particles is the filler or one of fillers, be between about 0.1 and 10 μm, or between about 1.0 μm and 5 μm. In certain aspects, where barium boroaluminosilicate glass particles is the filler or one of fillers, the median particle diameter may be about 0.6 μm, 0.8 μm, 1.0 μm, 1.2 μm, 1.4 μm, 1.6 μm, 1.8 μm, and 2.0 μm. The skilled artisan will understand that the particle size of the particular filler used will depend on the identity of the filler or fillers, and while the sizes provided here are with respect to barium boroaluminosilicate glass particles, similar sizes may pertain to one or more of the alternative fillers described herein.

When NACP is included as a filler, it will vary in size, but at least about 50, 55, 60, 65, 70, 75, 80, 85, 86, 87, 88, 89, 90, 91, 92, 93, 94, 95, 96, 97, 98 or 99% of the particles, or all of the particles (100%), have an average diameter of between about 10 nm and 500 nm. In certain aspects, the average diameter will be between about 25 nm and 400 nm, about 50 nm and 300 nm, about 75 nm and 200 nm, or about 100 nm and 150 nm. In a particular aspect, the NACP particles have an average diameter of between about 50 nm and 200 nm.

Depending on the identity of the filler, the particles comprising the filler may be silanized. Suitable means for silanization are known to the skilled artisan and include, but are not limited to, a mixture of about 4% 3-methacryloxypropyltrimethoxysilane and about 2% n-propylamine. In one embodiment, the filler comprises barium boroaluminosilicate glass particles, where the particles are silanized. In another embodiment, the filler comprises silanized barium boroaluminosilicate glass particles having a median particle diameter of about 1.4 μm.

In certain aspects, the protein-repellant dental composites of the present invention will comprise about 20% resin (whether dental resin, protein-repellant dental resin, or a mixture thereof) and about 80% filler by mass of the composite. In other aspects, the dental composites of the present invention will comprise about 21% resin and about 79% filler, 22% resin and about 78% filler, 23% resin and about 77% filler, 24% resin and about 76% filler, 25% resin and about 75% filler, 26% resin and about 74% filler, 27% resin and about 73% filler, 28% resin and about 72% filler, 29% resin and about 71% filler, 30% resin and about 70% filler, 31% resin and about 69% filler, 32% resin and about 68% filler, 33% resin and about 67% filler, 34% resin and about 66% filler, 35% resin and about 65% filler, 36% resin and about 64% filler, 37% resin and about 63% filler, 38% resin and about 62% filler, 39% resin and about 61% filler, or 40% resin and about 60% filler by mass of the composite.

The total amount of protein-repellant agent in the protein-repellant dental composite, regardless of whether the agent is added directly to the composite or whether it is encompassed in a protein-repellant dental resin used in the production of the composite, ranges from about 0.5% to 30% of the mass of the protein-repellant dental composite. In certain aspects, the range is from about 0.5% to 20%, about 0.5% to 15%, about 1% to 25%, about 1% to 20%, about 1% to 15%, about 1.5% to 20%, about 1.5% to 15%, about 1.5% to 10%, about 2% to 15%, about 2% to 10%, about 2% to 7.5%, about 2% to 5%, about 2.5% to 10%, about 2.5% to 7.5% or about 2.5% to 5% of the mass of the protein-repellant dental composite. In certain aspects, the amount of protein-repellant agent is about 0.5%, 1.0%, 1.5%, 2.0%, 2.5%, 3%, 3.5%, 4%, 4.5%, 5%, 5.5%, 6%, 6.5%, 7%, 7.5%, 8%, 8.5%, 9%, 9.5%, or 10%, of the mass of the protein-repellant dental composite.

In certain aspects, the protein-repellant dental composites of the invention may further comprise an antibacterial agent, a remineralizing agent, or both.

The antibacterial agents include, but are not limited to, antibacterial monomers, silver-containing nanoparticles (NAg), quaternary ammonium salts (QAS), chlorhexidine particles, TiO2 particles and ZnO particles.

The antibacterial monomers differ based on the length of the alkyl chain and include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM).

One or more antibacterial monomers may be present in the protein-repellant dental composites of the invention in a combined amount ranging from about 0.5% to 50% by mass of the protein-repellant dental composite. In certain aspects, the protein-repellant dental composite comprises a combined amount of from about 1% to 25%, from about 2.5% to 25%, from about 2.5% to 20%, from about 2.5% to 15%, from about 5% to 25%, from about 5% to 20%, from about 5% to 15%, from about 7.5% to 25%, from about 7.5% to 20%, about 7.5% to 15%, or from about 7.5% to 12.5% of the mass of the protein-repellant dental composite. In particular aspects, the amount of the one or more antibacterial monomers present in the protein-repellant dental composite is a combined amount of about 1, 2, 2.5, 3, 4, 5, 6, 7, 7.5, 8, 9, 10, 11, 12, 12.5, 13, 14, 15, 16, 17, 17.5, 18, 19, 20, 21, 22, 22.5, 23, 24, 25, 26, 27, 27.5, 28, 29, 30, 31, 32, 33, 34, 35, 36, 37, 38, 39, 40, 41, 42, 43, 44, 45, 46, 47, 48, 49 or 50% of the mass of the protein-repellant dental composite.

When present, suitable QASs include both polymerizable monomers and non-polymerizable small molecules, and include, but are not limited to, bis(2-methacryloyloxy-ethyl) dimethyl-ammonium bromide (QADM), methacryloyloxydodecylpyridinium bromide, methacryloxylethyl benzyl dimethyl ammonium chloride, methacryloxylethyl m-chloro benzyl dimethyl ammonium chloride, methacryloxylethyl cetyl dimethyl ammonium chloride, cetylpyridinium chloride, and methacryloxylethyl cetyl ammonium chloride, QAS chlorides, QAS bromides, QAS monomethacrylates, QAS dimethacrylates, and pre-fabricated QAS particles. The QAS may make up between about 1% and 30% of a mass fraction of the protein-repellant dental composite. In certain aspects, the QAS will make up between about 2% and 25%, about 5% and 20%, or about 7.5% and 15% of a mass fraction of the protein-repellant dental composite, or about 1%, 2.5%, 5%, 7.5%, 10%, 12.5, 15%, 17.5%, 20%, 22.5%, 25%, 27.5% or 30% of a mass fraction of the protein-repellant dental composite.

When present, suitable silver-containing nanoparticles (NAg) include, but are not limited to, silver 2-ethylhexanoate salt, silver-containing glass particles and silver benzoate. In addition to silver salts, pre-formed silver nanoparticles can be used. NAg may make up between about 0.01% and 20% of a mass fraction of the protein-repellant dental composite. In certain aspects, NAg will make up between about 0.05% and 5%, or about 0.08% and about 10%, of a mass fraction of the protein-repellant dental composite, or about 0.01%, 0.08%, 0.15%, 0.20%, 0.25%, 0.30%, 0.35%, 0.40%, 0.45%, 0.50%, 1.0%, 1.5%, 2.0%, 2.5%, 3.0%, 3.5%, 4.0%, 4.5% or 5.0% of a mass fraction of the protein-repellant dental composite. In one aspect, NAg makes up about 0.08% of a mass fraction of the protein-repellant dental composite. The silver particle size can range from about 1 nm to 1000 nm, and in one aspect, from about 2 nm to 500 nm.

In embodiments where the protein-repellant dental rein comprises a remineralizing agent, such agents include, but are not limited to, nanoparticles of amorphous calcium phosphate (NACP). NACP comprises nanometer-sized amorphous calcium phosphate (Ca₃[PO₄]₂) particles that can be used to produce a material with high Ca and PO₄ release, improved mechanical properties, and improved antibacterial properties. The materials that include NACP exhibit greatly increased ion release at acidic, cariogenic pH, when these ions are most needed to inhibit caries.

The NACP included in the protein-repellant dental composites of the present invention, when present, will vary in size, but at least about 50, 55, 60, 65, 70, 75, 80, 85, 86, 87, 88, 89, 90, 91, 92, 93, 94, 95, 96, 97, 98 or 99% of the particles, or all of the particles (100%), have an average diameter of between about 10 nm and 500 nm. In certain aspects, the average diameter will be between about 25 nm and 400 nm, about 50 nm and 300 nm, about 75 nm and 200 nm, or about 100 nm and 150 nm. In a particular aspect, the NACP particles have an average diameter of between about 50 nm and 200 nm.

The amount of NACP included in the protein-repellant dental composites may vary, but the NACP will generally comprise about 1% to 90% of the mass of the composite. In certain aspects, the NACP will comprise about 1% to 40%, about 5% to 70%, about 5% to 60%, about 5% to 50%, about 5% to 45%, about 5% to 40%, about 5% to 30%, about 10% to 90%, about 10% to 80%, about 10% to 70%, about 10% to 60%, about 10% to 50%, about 10% to 40%, about 15% to 70%, about 15% to 60%, about 15% to 50%, about 15% to 40%, about 20% to 90%, about 85% to 70%, about 20% to 80%, about 20% to 70%, about 20% to 60%, about 20% to 50%, about 20% to 40%, about 25% to 70%, about 25% to 60%, about 25% to 50%, about 25% to 40%, about 30% to 90%, about 30% to 85%, about 30% to 80%, about 30% to 75%, about 30% to 70%, about 30% to 60%, about 15% to 45%, about 15% to 35%, or about 15% to 25% of the mass of the resin. In certain other aspects, the NACP is a mass fraction of about 25%, 25.5%, 26%, 26.5%, 27%, 27.5%, 28%, 28.5%, 29%, 29.5% or 30% of the composite. In particular aspects, the NACP will comprise about 5, 6, 7, 8, 9, 10, 11, 12, 13, 14, 15, 16, 17, 18, 19, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90% of the mass of the composite. In certain aspects, the NACP will range from about 10% to 40% of the mass of the composite.

In non-limiting examples of the present invention, the protein-repellant dental composite comprises:

(i) about 70% by mass filler, about 25% by mass dental resin, about 3% by mass protein-repellant agent, and about 2% by mass antibacterial agent;
(ii) about 70% by mass filler, about 25% by mass dental resin, about 2.5% by mass protein-repellant agent, and about 2.5% by mass antibacterial agent; and
(iii) about 70% by mass filler, about 25% by mass dental resin, about 3.5% by mass protein-repellant agent, and about 1.5% by mass antibacterial agent.

In further non-limiting examples of the present invention, the protein-repellant dental composite comprises:

(i) about 70% by mass barium boroaluminosilicate glass filler, about 25% by mass BisGMA-TEGDMA (1:1 mass ratio) dental resin, about 3% by mass protein-repellant agent, and about 2% by mass antibacterial agent, wherein the antibacterial agent is DMADDM, DMAPDM, or DMAHDM;
(ii) about 70% by mass barium boroaluminosilicate glass filler, about 25% by mass BisGMA-TEGDMA (1:1 mass ratio) dental resin, about 2.5% by mass protein-repellant agent, and about 2.5% by mass antibacterial agent, wherein the antibacterial agent is DMADDM, DMAPDM, or DMAHDM; and
(iii) about 70% by mass barium boroaluminosilicate glass filler, about 25% by mass BisGMA-TEGDMA (1:1 mass ratio) dental resin, about 3.5% by mass protein-repellant agent, and about 1.5% by mass antibacterial agent, wherein the antibacterial agent is DMADDM, DMAPDM, or DMAHDM.

In still further non-limiting examples of the present invention, the protein-repellant dental composite comprises:

(i) about 50% by mass barium boroaluminosilicate glass filler, about 20% NACP, about 25% by mass BisGMA-TEGDMA (1:1 mass ratio) dental resin, about 3% by mass protein-repellant agent, and about 2% by mass antibacterial agent, wherein the antibacterial agent is DMADDM, DMAPDM, or DMAHDM;
(ii) about 50% by mass barium boroaluminosilicate glass filler, about 20% NACP, about 25% by mass BisGMA-TEGDMA (1:1 mass ratio) dental resin, about 2.5% by mass protein-repellant agent, and about 2.5% by mass antibacterial agent, wherein the antibacterial agent is DMADDM, DMAPDM, or DMAHDM; and
(iii) about 50% by mass barium boroaluminosilicate glass filler, about 20% NACP, about 25% by mass BisGMA-TEGDMA (1:1 mass ratio) dental resin, about 3.5% by mass protein-repellant agent, and about 1.5% by mass antibacterial agent, wherein the antibacterial agent is DMADDM, DMAPDM, or DMAHDM.

The dental materials of the invention, including the protein-repellant dental primers, protein-repellant dental adhesives, protein-repellant dental resins and protein-repellant dental composites, are suitable for use in the teeth of mammals, including primates such as human or non-human primates, and those of dogs, cats, horses, cattle, pigs, goats and sheep, for example.

### Dental Bonding System

The protein-repellent dental bonding systems of the invention include a three-component system comprising a dental primer, a dental adhesive and an etchant; a two-component system comprising a dental primer and a dental adhesive; a three-step bonding system; a two-step bonding system; and a one-step self-adhesive bonding system.

The three-component protein-repellent dental bonding system comprises (i) an etchant, (ii) a dental primer, and (iii) a dental adhesive, wherein the dental primer is a protein-repellant dental primer, or the dental adhesive is a protein-repellant agent dental adhesive, or the dental primer is a protein-repellant dental primer and the dental adhesive is a protein-repellant agent dental adhesive. This system comprises the etchant, the protein-repellant dental primer and the protein-repellant dental adhesive as separate components to be used in tandem in the system. For example, the etchant is applied to a selected surface, followed by application of protein-repellant dental primer, and then the protein-repellant dental adhesive to the same surface.

The two-component protein-repellent dental bonding system comprises (i) a dental primer and (ii) a dental adhesive, wherein the dental primer is a protein-repellant dental primer, or the dental adhesive is a protein-repellant agent dental adhesive, or the dental primer is a protein-repellant dental primer and the dental adhesive is a protein-repellant agent dental adhesive. This system comprises the protein-repellant dental primer and the protein-repellant dental adhesive as separate components to be used in tandem in the system. For example, the protein-repellant dental primer is applied to a selected surface, followed by application of the protein-repellant dental adhesive to the same surface.

The two-component protein-repellent dental bonding system may be used, in one example, as follows. Before using the bonding system, the tooth is prepared by removing decayed material, cleaning the enamel/dentin surface and applying an etchant to the enamel/dentin surface. A bonding system is then utilized, which can comprise, as an example, application of a dental primer, followed by a dental adhesive. After curing the adhesive, a composite is packed into the void of the tooth. In some aspects, the dental primer or the dental adhesive in the two-component dental bonding systems is a protein-repellent dental primer and/or a protein-repellent dental adhesive.

The three-step protein-repellent dental bonding system comprises (i) an etchant, (ii) a dental primer, and (iii) a dental adhesive, wherein the dental primer is a protein-repellant dental primer, or the dental adhesive is a protein-repellant agent dental adhesive, or the dental primer is a protein-repellant dental primer and the dental adhesive is a protein-repellant agent dental adhesive. This system comprises the etchant, the protein-repellant dental primer and the protein-repellant dental adhesive as separate components to be used in tandem in the system. For example, in a first step the etchant is applied to a selected surface, followed by application of protein-repellant dental primer in a second step, and then the protein-repellant dental adhesive to the same surface in a third step.

The two-step protein-repellent dental bonding system comprises (i) an etchant, and (ii) a mixture comprising a dental primer and a dental adhesive, wherein the dental primer is a protein-repellant dental primer, or the dental adhesive is a protein-repellant agent dental adhesive, or the dental primer is a protein-repellant dental primer and the dental adhesive is a protein-repellant agent dental adhesive. This system comprises the etchant and the primer-dental mixture as separate components to be used in tandem in the system. For example, in a first step the etchant is applied to a selected surface, followed by application of primer-mixture in a second step to the same surface.

The one-step self-adhesive protein-repellent bonding system comprises a mixture that comprises an etchant, a dental primer, and a dental adhesive, wherein the dental primer is a protein-repellant dental primer, or the dental adhesive is a protein-repellant agent dental adhesive, or the dental primer is a protein-repellant dental primer and the dental adhesive is a protein-repellant agent dental adhesive.

The dental primers, dental adhesives, protein-repellant dental primers and protein-repellant dental adhesives for use in these systems are as defined herein.

Suitable etchants for use in the dental bonding systems of the present invention include Scotchbond Multi-Purpose (SBMP) (3M, St. Paul, Minn.) etchant which contains 37% phosphoric acid.

### Adhesive Coating System

The invention includes a protein-repellant adhesive coating system comprising (i) a protein-repellant dental primer and (ii) a protein-repellant dental adhesive, wherein the protein-repellant dental adhesive comprises NACP. The system comprises the protein-repellant dental primer and the protein-repellant dental adhesive as separate components to be used in tandem in the system. For example, the protein-repellant dental primer is applied to a selected surface, followed by application of the protein-repellant dental adhesive to the same surface.

In certain aspects, the protein-repellant dental primer or the protein-repellant dental adhesive, or both may further comprise an antibacterial agent.

The protein-repellant dental primer and protein-repellant dental adhesive for use in the protein-repellant adhesive coating system are as defined herein.

The amount of protein-repellant agent in the protein-repellant dental primer ranges from about 0.5% to 50% of the mass of the protein-repellant dental primer. In certain aspects, the range is from about 1% to 25%, about 2.5% to 20%, about 4% to 15%, or about 5% to 12.5% of the mass of the protein-repellant dental primer. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10%, of the mass of the protein-repellant dental primer.

The amount of protein-repellant agent in the protein-repellant dental adhesive ranges from about 0.5% to 50% of the mass of the protein-repellant dental adhesive. In certain aspects, the range is from about 1% to 25%, about 2.5% to 20%, about 4% to 15%, or about 5% to 12.5% of the mass of the protein-repellant dental adhesive. In certain aspects, the amount of protein-repellant agent is about 2.5%, 3%, 3.5%, 4%, 4.5%, 5%, 5.5%, 6%, 6.5%, 7%, 7.5%, 8%, 8.5%, 9%, 9.5%, or 10%, of the mass of the protein-repellant dental adhesive.

The protein-repellant dental adhesive comprises between about 10% to 50% NACP by mass of the protein-repellant dental adhesive. In particular aspects, the mass fraction of NACP in the dental adhesive ranges from about 15% to 40% or about 20% to 35%. In certain aspects, the mass fraction of NACP in the dental adhesive is about 15%, 20%, 25%, 30%, 35% or 40%.

When present, the antibacterial agent may be a single antibacterial agent or a mixture of two or more antibacterial agents. The antibacterial agents include, but are not limited to, antibacterial monomers, silver-containing nanoparticles (NAg), quaternary ammonium salts (QAS), chlorhexidine particles, TiO₂ particles and ZnO particles.

The antibacterial monomers include dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM). In certain aspects, the antibacterial monomer is DMADDM, DMATDM, DMATTDM, DMAPDM or DMAHDM.

When used, the amount of antibacterial monomers present in the protein-repellant dental primer or protein-repellant dental adhesive is independently from about 0.5% to 50% of the material (i.e., protein-repellant dental primer and protein-repellant dental adhesive), preferably from about 2% to 20% of the mass of the material. In certain aspects, the antibacterial monomers can be incorporated into the material at antibacterial monomer mass fractions ranging from about 1% to 50%, from about 1% to 25%, from about 1.5% to 20%, from about 2% to 20%, from about 2% to 15%, from about 2.5% to 25%, from about 2.5% to 20%, from about 2.5% to 15%, from about 2.5% to 12.5%, from about 5% to 25%, from about 5% to 20%, from about 5% to 15%, from about 5% to 10%, from about 7.5% to 25%, from about 7.5% to 20%, from about 7.5% to 15%, or from about 7.5% to 12.5% of the mass of the material. In particular aspects, the amount of the antibacterial monomers present in the material is a combined amount of about 0.5, 1, 2, 2.5, 3, 4, 5, 6, 7, 7.5, 8, 9, 10, 11, 12, 12.5, 13, 14, 15, 16, 17, 17.5, 18, 19, 20, 21, 22, 22.5, 23, 24, 25, 26, 27, 27.5, 28, 29, 30, 31, 32, 33, 34, 35, 36, 37, 38, 39, 40, 41, 42, 43, 44, 45, 46, 47, 48, 49 or 50% of the mass of the material. In certain aspects of the invention, the combined amount of the antibacterial monomers incorporated into the material ranges from about 1% to 50% of the mass of the material. In particular aspects, the combined amount of the antibacterial monomers present in the material is about 10%, 7.5%, or 5% of the mass of the material. In certain aspects, one of the antibacterial monomers identified herein is included in the material. In certain other aspects, two, three, four, or more of the antibacterial monomers identified herein are included in the material.

When used, the amount of quaternary ammonium salts present in the protein-repellant dental primer or protein-repellant dental adhesive independently ranges from about 1% to 30% of the mass of the protein-repellant dental primer or protein-repellant dental adhesive, preferably about 7.5% to 15%.

When used, the amount of silver-containing nanoparticles, chlorhexidine particles, TiO2 particles or ZnO particles present in the protein-repellant dental primer or protein-repellant dental adhesive independently ranges from about 0.01% and 20% of the mass of the protein-repellant dental primer or protein-repellant dental adhesive, preferably about 0.08% to 10% of the mass of the protein-repellant dental primer or protein-repellant dental adhesive.

When used, the amount of silver-containing nanoparticles, chlorhexidine particles, TiO2 particles or ZnO particles present in the protein-repellant dental primer or protein-repellant dental adhesive independently ranges from about 0.01% and 20% of the mass of the protein-repellant dental primer or protein-repellant dental adhesive, preferably about 0.08% to 10% of the mass of the protein-repellant dental primer or protein-repellant dental adhesive.

In addition to the protein-repellent dental primers, dental adhesives, dental resins, dental composites, and dental bonding systems discussed above, the invention is directed to protein-repellant dental cements, dental sealants, dental bases and dental liners, each of which is protein-repellent and each of which comprises a protein-repellent agent. The skilled artisan will be able to easily envision the composition of these additional dental materials in light of the teachings provided herein. It can also be noted that the dental cements include crown cements, orthodontic bracket cements, glass ionomer cements, resin-modified glass ionomer cements and other fluoride-releasing materials, that can be made protein-repellent and antibacterial.

### Methods

The dental materials described herein can be used in a method of inhibiting growth of aciduric bacteria on a surface of a tooth of a subject, comprising restoring a surface of the tooth from which a decayed portion has been removed by applying a protein-repellant dental composite as described herein to the surface of the tooth, thereby inhibiting growth of aciduric bacteria on the tooth of the subject.

The dental materials described herein can also be used in a method of inhibiting further decay of a decaying tooth in a subject, comprising restoring a surface of the tooth from which a decayed portion has been removed by applying a protein-repellant dental composite as described herein to the surface of the tooth, thereby inhibiting further decay of the decaying tooth in the subject.

The protein-repellant dental bonding systems, protein-repellant dental primers and protein-repellant dental adhesives described herein can be used in a method of inhibiting growth of aciduric bacteria on a surface of a tooth of a subject. Such methods comprise restoring a surface of a tooth from which a decayed portion has been removed by applying a protein-repellant dental composite using a protein-repellant dental bonding system described herein to the surface of the tooth, thereby inhibiting growth of aciduric bacteria on the tooth of the subject. Such methods also comprise restoring a surface of a tooth from which a decayed portion has been removed by applying a protein-repellant dental composite using a protein-repellant dental primer and/or protein-repellant dental adhesive described herein to the surface of the tooth, thereby inhibiting growth of aciduric bacteria on the tooth of the subject.

The protein-repellant dental bonding systems, protein-repellant dental primers and protein-repellant dental adhesives described herein can also be used in a method of inhibiting further decay of a decaying tooth in a subject. Such methods comprise restoring a surface of the tooth from which a decayed portion has been removed by applying a protein-repellant dental composite using a protein-repellant dental bonding system described herein to the surface of the tooth, thereby inhibiting further decay of the decaying tooth in the subject. Such methods also comprise restoring a surface of the tooth from which a decayed portion has been removed by applying a protein-repellant dental composite using a protein-repellant dental primer and/or protein-repellant dental adhesive described herein to the surface of the tooth, thereby inhibiting further decay of the decaying tooth in the subject.

### EXAMPLES

### Example 1: Novel Protein-Repellent Dental Adhesive Containing 2-Methacryloyloxyethyl Phosphorylcholine

### Materials and Methods

### 1.1. Fabrication of Protein-Repellent Bonding Agent

The primer and adhesive from the Scotchbond Multi-Purpose (SBMP, 3M, St. Paul, Minn.) system were used. According to the manufacturer, SBMP primer contained 35-45% of HEMA, 10-20% of a copolymer of acrylic and itaconic acids, and 40-50% water. SBMP adhesive contained 60-70% of bisphenol A diglycidyl methacrylate (BisGMA) and 30-40% of 2-hydroxyethyl methacrylate (HEMA), tertiary amines and photo-initiator.

2-methacryloyloxyethyl phosphorylcholine (MPC; Sigma-Aldrich, St. Louis, Mo.) was commercially available which was synthesized via a method reported by Ishihara et al., 1990. The MPC powder was mixed with SBMP primer at MPC/(SBMP primer+MPC) mass fractions of 0%, 3.75%, 7.5%, 11.25% and 15%. The 7.5% was selected following previous studies (Moro et al., 2009; Sibarani et al., 2007). The other mass fractions enabled the investigation of the relationship between MPC mass fraction and protein-repellent efficacy in a dental resin. MPC mass fractions higher than 15% were not included due to a decrease in the dentin bond strength. Each batch of primer was magnetically-stirred with a bar at a speed of 150 rpm (Bellco Glass, Vineland, N.J.) for 24 h to completely dissolve the MPC in the SBMP primer.

Similarly, MPC was mixed into SBMP adhesive at the same mass fractions. Hence, five bonding agents were tested:

(1) SBMP primer and adhesive control;
(2) SBMP primer+3.75% MPC, SBMP adhesive+3.75% MPC (termed “3.75% MPC”);
(3) SBMP primer+7.5% MPC, SBMP adhesive+7.5% MPC (“7.5% MPC”);
(4) SBMP primer+11.25% MPC, SBMP adhesive+11.25% MPC (“11.25% MPC”);
(5) SBMP primer+15% MPC, SBMP adhesive+15% MPC (“15% MPC”).

For protein adsorption and biofilm testing, resin disks were prepared following previous studies (Imazato et al., 2003c; Zhou et al., 2013a). Briefly, the cover of a 96-well plate was used as molds. Ten μL of a primer was placed in the bottom of each dent. After drying with a stream of air, 20 μL of adhesive was applied to the dent and photo-polymerized for 30 s using a quartz-tungsten-halogen light-curing unit (Demetron VCL 401, Demetron, CA) with output intensity of 600 mW/cm², using a mylar strip covering to obtain a disk of approximately 8 mm in diameter and 0.5 mm in thickness. The cured disks were immersed in 200 mL of distilled water and magnetically-stirred with a bar at a speed of 100 rpm for 1 h to remove any uncured monomers, following a previous study (Imazato et al., 1998).

### 1.2. Dentine Shear Bond Testing

Extracted caries-free human molars were sawed to remove the crowns (Isomet, Buehler, Lake Bluff, Ill.), then ground perpendicularly to the longitudinal axis on 320 grit SiC paper until occlusal enamel was completely removed (Cheng et al., 2012; Zhang et al., 2012). The dentin surface was etched for 15 s and rinsed with water (Antonucci et al., 2009). A primer was applied, and the solvent was removed with an air stream. An adhesive was applied and light-cured for 10 s. A stainless-steel iris, having a central opening with a diameter of 4 mm and a thickness of 1.5 mm, was held against the adhesive-treated dentin surface. The central opening was filled with a composite (TPH, Caulk/Dentsply, Milford, Del.) and light-cured for 60 s (Antonucci et al., 2009).

The bonded specimens were stored in distilled water at 37° C. for 24 h. Dentin shear bond strength, S_{D}, was measured as previously described (Cheng et al., 2012; Antonucci et al., 2009). Briefly, a chisel was held parallel to the composite-dentine interface and loaded via a Universal Testing Machine (MTS, Eden Prairie, Minn.) at 0.5 mm/min until the composite-dentine bond failed. S_{D} was calculated as: S_{D}=4P/(πd²), where P is the load at failure, and d is the diameter of the composite (Cheng et al., 2012; Antonucci et al., 2009). Ten teeth were tested for each of the aforementioned five groups.

### 1.3. Characterization of Protein Adsorption by Micro Bicinchoninic Acid Method

The amount of protein adsorbed on resin disks was determined by the micro bicinchoninic acid (BCA) method (Moro et al., 2009; Sibarani et al., 2007; Takahashi et al., 2014). Each disk was immersed in phosphate buffered saline (PBS) for 2 h before immersing in 4.5 g/L bovine serum albumin (BSA) (Sigma-Aldrich) solutions at 37° C. for 2 h (Moro et al., 2009; Sibarani et al., 2007). The disks then were rinsed with fresh PBS by stirring method (300 rpm for 5 min). The adsorbed protein was detached in sodium dodecyl sulfate (SDS) 1 wt % in PBS by sonication for 20 min. A protein analysis kit (micro BCA protein assay kit, Fisher Scientific, Pittsburgh, Pa.) was used to determine the BSA concentration in the SDS solution. From the concentration of protein, the amount of protein adsorbed on the resin disk surface was calculated (Moro et al., 2009; Sibarani et al., 2007). Six disks were evaluated for each group.

### 1.4. Dental Plaque Microcosm Biofilm Model

A dental plaque microcosm biofilm model using human saliva was used to test the protein-repellent resins (Cheng et al., 2012; Zhang et al., 2012). Saliva is ideal for growing microcosm biofilms in vitro, with the advantage of maintaining much of the complexity and heterogeneity of the dental plaque in vivo (McBain, 2009). Saliva was collected from ten healthy donors having natural dentition without active caries, and not having used antibiotics within the preceding 3 months. The donors did not brush teeth for 24 h and abstained from food and drink intake for 2 h prior to donating saliva. Stimulated saliva was collected during parafilm chewing and was kept on ice. An equal volume of saliva from each of the ten donors was combined to form the saliva sample. The saliva was diluted in sterile glycerol to a saliva concentration of 70%, and stored at −80° C. for subsequent use (Cheng et al., 2011).

The saliva-glycerol stock was added, with 1:50 dilution, into the growth medium as inoculum. The growth medium contained mucin (type II, porcine, gastric) at a concentration of 2.5 g/L; bacteriological peptone, 2.0 g/L; tryptone, 2.0 g/L; yeast extract, 1.0 g/L; NaCl, 0.35 g/L, KCl, 0.2 g/L; CaCl₂, 0.2 g/L; cysteine hydrochloride, 0.1 g/L; hemin, 0.001 g/L; vitamin K1, 0.0002 g/L, at pH 7 (McBain et al., 2005). The resin disks were sterilized in ethylene oxide (Anprolene AN 74i, Andersen, Haw River, N.C.). Then, 1.5 mL of inoculum was added to each well of 24-well plates containing a resin disk, and incubated at 37° C. in 5% CO₂ for 8 h. Then, the disks were transferred to new 24-well plates filled with fresh medium and incubated. After 16 h, the disks were transferred to new 24-well plates with fresh medium and incubated for 24 h. This totaled 48 h of incubation, which was adequate to form plaque microcosm biofilms as shown in previous study (Cheng et al., 2011).

### 1.5. Live/Dead Assay

Resin disks with 2-day biofilms were washed with PBS and stained using the BacLight live/dead kit (Molecular Probes, Eugene, Oreg.) (Cheng et al., 2012; Zhang et al., 2012). Live bacteria were stained with Syto 9 to produce a green fluorescence. Bacteria with compromised membranes were stained with propidium iodide to produce a red fluorescence. The stained disks were examined using an inverted epifluorescence microscope (Eclipse TE2000-S, Nikon, Melville, N.Y.). Six disks were evaluated for each group. Three randomly chosen fields of view were photographed from each disk, yielding a total of 18 images for each group.

### 1.6. MTT Assay of Metabolic Activity

The MTT (3-[4,5-dimethylthiazol-2-yl]-2,5-diphenyltetrazolium bromide) assay was used to examine the metabolic activity of the 2-day biofilms on resin disks (Cheng et al., 2012; Zhang et al., 2012). MTT is a colorimetric assay that measures the enzymatic reduction of MTT, a yellow tetrazole, to formazan. Disks with 2-day biofilms (n=6) were transferred to a new 24-well plate, then 1 mL of MTT dye (0.5 mg/mL MTT in PBS) was added to each well and incubated at 37° C. in 5% CO₂ for 1 h. During this process, metabolically active bacteria reduced the MTT to purple formazan. After 1 h, the disks were transferred to a new 24-well plate, 1 mL of dimethyl sulfoxide (DMSO) was added to solubilize the formazan crystals. The plate was incubated for 20 min with gentle mixing at room temperature in the dark. Then, 200 μL of the DMSO solution from each well was collected, and its absorbance at 540 nm was measured via a microplate reader (SpectraMax M5, Molecular Devices, Sunnyvale, Calif.). A higher absorbance is related to a higher formazan concentration, which indicates a higher metabolic activity in the biofilm on the disk (Cheng et al., 2012; Zhang et al., 2012).

### 1.7. Colony Forming Unit (CFU) Counts

Resin disks with 2-day biofilms were rinsed with PBS to remove loose bacteria (Cheng et al., 2012; Zhang et al., 2012). Then the disks were transferred into tubes with 2 mL PBS, and the biofilms were harvested by sonication (3510RMTH, Branson, Danbury, Conn.) for 5 min, followed by vortexing at 2400 rpm for 30 s using a vortex mixer (Fisher Scientific) (Cheng et al., 2012; Zhang et al., 2012). Three types of agar plates were examined: First, tryptic soy blood plates were used to determine total micro-organisms (Cheng et al., 2011). Second, mitis salivarius agar (MSA) culture plates plus 15% sucrose were used to determine total streptococci (Lima et al., 2009). This is because MSA contains selective agents including crystal violet, potassium tellurite and trypan blue, which inhibit most Gram-negative bacilli and most Gram-positive bacteria except streptococci, thus enabling the streptococci to grow (Lima et al., 2009). Third, cariogenic mutans streptococci is known to be resistant to bacitracin, and this property is used to isolate mutans streptococci from the highly heterogeneous oral microflora (Cheng et al., 2011). Therefore, MSA plus 0.2 units of bacitracin per mL was used to determine mutans streptococci (Cheng et al., 2011). The purpose of measuring these three types of CFU counts was to provide bacterial adhesion and viability on not only the total microorganisms, but also mutans streptococci, in the dental plaque microcosm biofilms. The mutans streptococci group consists of mutans streptococcus and sobrinus streptococcus, both species playing a key role in dental caries. The bacterial suspensions were serially diluted, spread onto agar plates and incubated at 37° C. in 5% CO₂ for 24 h. The number of colonies that grew was counted and used, along with the dilution factor, to calculate total CFU counts on each disk (Cheng et al., 2012; Zhang et al., 2012).

### 1.8. Statistical Analysis

One-way and two-way analyses of variance (ANOVA) were performed to detect the significant effects of the variables. Tukey's multiple comparison test was used to compare the data at a p value of 0.05.

### Results

FIG. 1 plots the dentin shear bond strength results (mean±sd; n=10). Increasing the MPC mass fraction to 11.25% and 15% MPC caused a decrease in dentin bond strength (p<0.05). However, the SBMP bonding agents with 3.75% and 7.5% MPC had similar bond strengths to control (p>0.1). At 7.5% MPC, the dentin bond strength was (30±2.8) MPa, not significantly different from the (33±3.6) MPa of SBMP control (p>0.1).

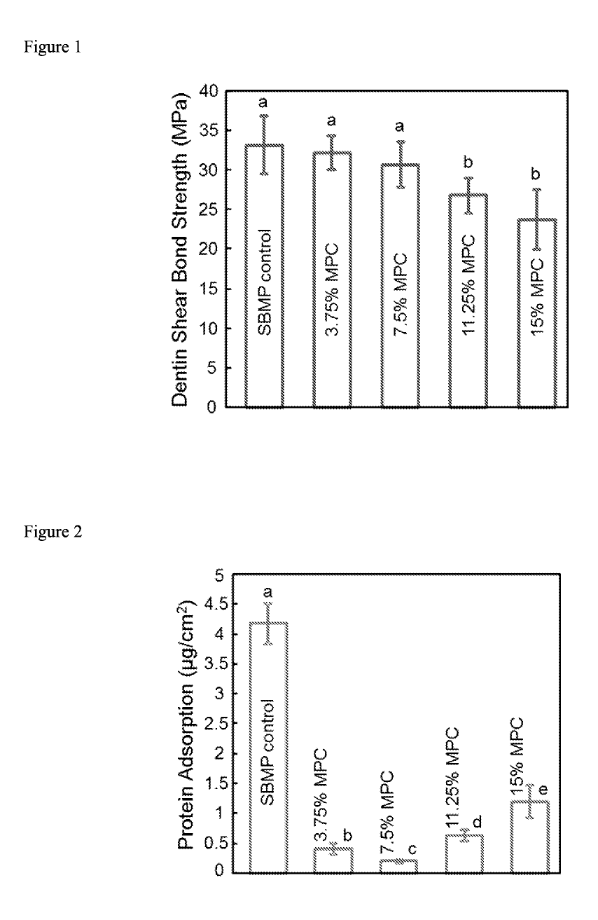

The amounts of protein adsorption on resin disks are plotted in FIG. 2 (mean±sd; n=6). Incorporation of MPC into primer and adhesive greatly decreased the amount of protein adsorption, reaching a minimum at 7.5% MPC. Further increasing the MPC mass fraction increased the protein adsorption to the resin surface. These results showed that the resin with 7.5% MPC had the lowest amount of protein adsorption, which was nearly 20-fold less than that of SBMP control.

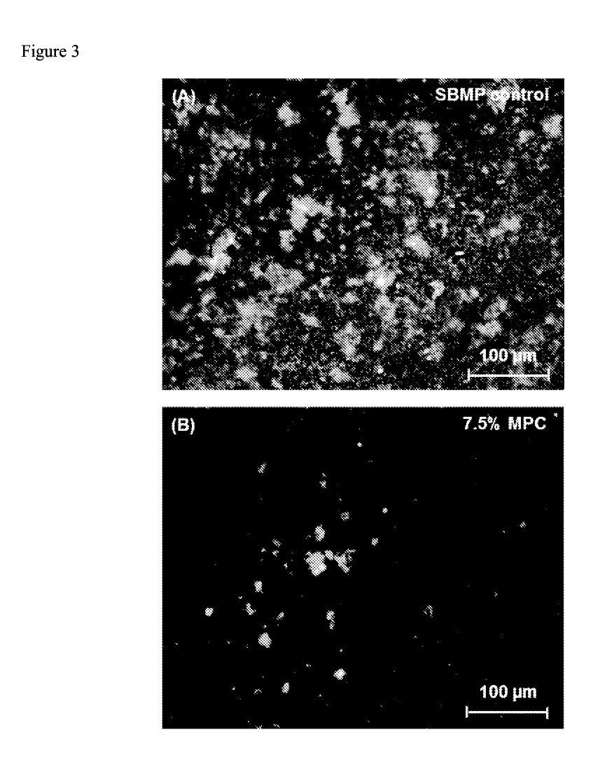

A dental plaque microcosm biofilm model was used with human saliva as inoculum. FIG. 3 shows representative live/dead staining images of 2-day biofilms grown on disks of SBMP control and SBMP containing 7.5% MPC. Live bacteria were stained green, and dead bacteria were stained red. Disks of SBMP control and SBMP containing 7.5% MPC both had primarily live bacteria. However, SBMP control disks were covered with green biofilms. In contrast, SBMP containing 7.5% MPC had much less bacterial adhesion and biofilm coverage on the disks.

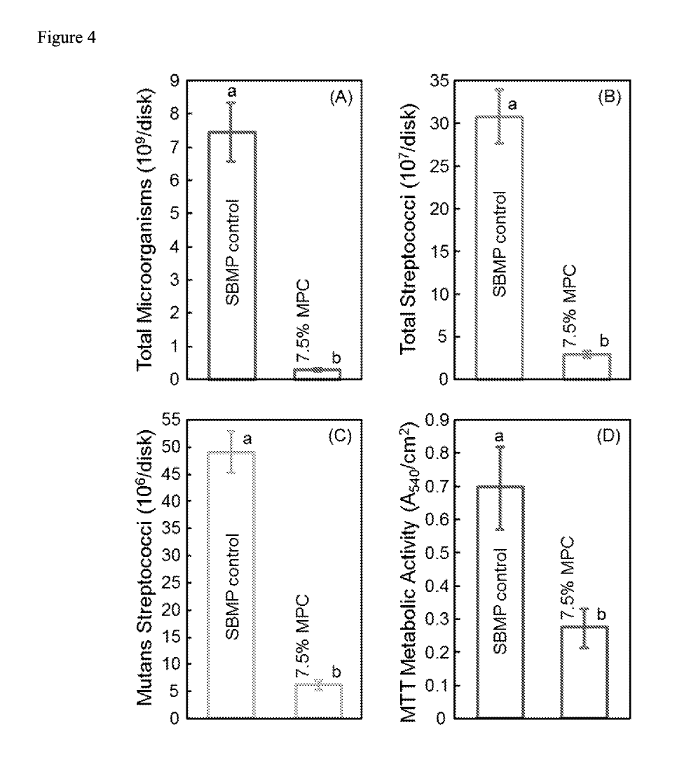

FIG. 4 plots the quantitative 2-day biofilm response on disks of SBMP control and SBMP plus 7.5% MPC: (A) Total microorganisms, (B) total streptococci, (C) mutans streptococci, and (D) metabolic activity (mean±sd; n=6). Incorporation of MPC into primer and adhesive greatly reduced all three CFU counts and the metabolic activity of biofilms, compared to SBMP control (p<0.05). All three CFU counts of biofilms on resin disks with 7.5% MPC were an order of magnitude lower than those of SBMP control.

The results demonstrate that protein adsorption was reduced on the adhesive resin by an order of magnitude, which in turn reduced oral biofilm CFU by an order of magnitude. The inhibition of bacteria attachment and biofilm growth was achieved without compromising the dentin shear bond strength of the protein-repellent adhesive, compared to the unmodified commercial bonding agent control.

The results of the protein adsorption assay confirmed that the incorporation of MPC into primer and adhesive greatly decreased the protein adsorption. While the resin with 7.5% MPC had the least protein adsorption, the protein adsorption increased at MPC mass fractions of 11.25% or higher. A possible reason is that, when MPC mass fractions were relatively high, the MPC powder could hardly be dissolved in the SBMP primer or adhesive completely. The excess of MPC might decrease the protein-repellent efficacy and also adversely affect the dentin bond strength. Indeed, the SBMP bonding agents with high MPC mass fractions (>11.25%) had significantly lower dentin bond strength. In contrast, the bonding agent with 7.5% MPC had a dentin bond strength similar to that of control (p>0.1). Taken together, these findings indicate that the use of an optimal MPC mass fraction in the bonding agent is essential to achieving the maximal protein-repellent ability and dentin bond strength. In the present study, incorporation of 7.5% MPC into SBMP primer and adhesive appeared to be optimal in obtaining the highest protein-repellent efficacy, without adversely affecting the dentin bond strength.

Proteins adsorbed onto the resin surface in the oral environment provide a medium for the early attachment of bacteria and microorganisms, thereby initiating the basis for biofilm formation (Kolenbrander et al., 1993; Donlan et al., 2002). Therefore, the fact that the MPC-containing adhesive resin can repel proteins (FIG. 2) suggests that this resin could also inhibit biofilm growth. Indeed, the results in FIGS. 3 and 4 confirmed that the incorporation of MPC at mass fraction of 7.5% into primer and adhesive greatly reduced bacteria adhesion, biofilm CFU and metabolic activity, compared to the commercial bonding agent control. These results demonstrate that protein-repellent dental resins are a promising approach in combating biofilms and inhibiting secondary caries.

### Example 2: Development of Novel Dental Adhesive with Double Benefits of Protein-Repellent and Antibacterial Capabilities

### Materials and Methods

2.1. Incorporation of MPC into Bonding Agent

Scotchbond Multi-Purpose Adhesive and Primer (referred as “SBMP”) (3M, St. Paul, Minn.) were used as the parent bonding system. According to the manufacturer, SBMP primer contained 35-45% of HEMA, 10-20% of a copolymer of acrylic and itaconic acids, and 40-50% water. SBMP adhesive contained 60-70% of bisphenol A diglycidyl methacrylate (BisGMA) and 30-40% of 2-hydroxyethyl methacrylate (HEMA), tertiary amines and photo-initiator. The protein-repellent monomer, 2-methacryloyloxyethyl phosphorylcholine (MPC), was synthesized via a method reported by Ishihara et al., 1990 and commercially available (Sigma-Aldrich, St. Louis, Mo.). The MPC powder was mixed with SBMP primer at MPC/(SBMP primer+MPC) of 7.5% by mass. The 7.5% mass fraction was selected following our preliminary study, which showed that 7.5% MPC in the resin yielded the strongest protein-repellent property without compromising the dentin shear bond strength. The primer with MPC was magnetically-stirred with a bar at a speed of 150 rpm (Bellco Glass, Vineland, N.J.) for 24 h to completely dissolve MPC in primer. Similarly, 7.5% MPC by mass was also mixed into the SBMP adhesive.

2.2. Incorporation of DMAHDM into Bonding Agent

Dimethylaminododecyl methacrylate (DMAHDM) with an alkyl chain length of 16 was synthesized using a modified Menschutkin reaction following previous studies (Zhou et al., 2013a; Antonucci et al., 2012; Cheng et al., 2012a; Cheng et al., 2013a). In this method, a tertiary amine group was reacted with an organo-halide. A benefit of this reaction is that the reaction products are generated at virtually quantitative amounts and require minimal purification. Briefly, 10 mmol of 1-(dimethylamino)docecane (Sigma-Aldrich) and 10 mmol of 1-bromohexadecane (BHD, TCI America, Portland, Oreg.) were combined with 3 g of ethanol in a 20 mL scintillation vial. The vial was stirred at 70° C. for 24 h. The solvent was then removed via evaporation, yielding DMAHDM as a clear, colorless, and viscous liquid (Zhou et al., 2013a).

SBMP primer was first mixed with MPC as described above. Then DMAHDM was mixed into the SBMP-MPC primer, at DMAHDM/(SBMP primer+DMAHDM) mass fractions of 5%, 7.5%, and 10%, respectively. These DMAHDM mass fractions followed previous study (Zhou et al., 2013a). Similarly, DMAHDM was incorporated into the SBMP-MPC adhesive at the same mass fractions. The following eight bonding systems were investigated:
(1) SBMP primer and adhesive control (termed “SBMP control”);
(2) SBMP primer+7.5% MPC, SBMP adhesive+7.5% MPC (“7.5% MPC”);
(3) SBMP primer+5% DMAHDM, SBMP adhesive+5% DMAHDM (“5% DMAHDM”);
(4) SBMP primer+7.5% DMAHDM, SBMP adhesive+7.5% DMAHDM (“7.5% DMAHDM”);
(5) SBMP primer+10% DMAHDM, SBMP adhesive+10% DMAHDM (“10% DMAHDM”);
(6) SBMP primer+7.5% MPC+5% DMAHDM, SBMP adhesive+7.5% MPC+5% DMAHDM (“7.5% MPC+5% DMAHDM”);
(7) SBMP primer+7.5% MPC+7.5% DMAHDM, SBMP adhesive+7.5% MPC+7.5% DMAHDM (“7.5% MPC+7.5% DMAHDM”);
(8) SBMP primer+7.5% MPC+10% DMAHDM, SBMP adhesive+7.5% MPC+10% DMAHDM (“7.5% MPC+10% DMAHDM”).

### 2.3. Dentin Shear Bond Testing

The use of extracted human teeth was approved by the University of Maryland. Extracted caries-free molars were first sawed to remove the crowns (Isomet, Buehler, Lake Bluff, Ill.), then ground perpendicularly to their longitudinal axes on 320 grit SiC paper until occlusal enamel was completely removed (Cheng et al., 2012b; Zhang et al., 2012). The dentin surface was etched with etchant for 15 s and rinsed with water (Antonucci et al., 2009). A primer was applied, and the solvent was removed with an air stream. The adhesive was applied and light-cured for 10 s (Optilux-VCL401, Demetron, Danbury, Conn.). A stainless-steel iris, having a central opening with a diameter of 4 mm and a thickness of 1.5 mm, was held against the adhesive-treated dentin surface. The central opening was filled with a composite (TPH, Caulk/Dentsply, Milford, Del.), and light-cured for 60 s (Antonucci et al., 2009). The bonded specimens were stored in distilled water at 37° C. for 24 h (Cheng et al., 2012b). The dentin shear bond strength, S_{D}, was measured as previously described (Cheng et al., 2012b; Antonucci et al., 2009). A chisel was held parallel to the composite-dentine interface and loaded via a Universal Testing Machine (MTS, Eden Prairie, Minn.) at 0.5 mm/min until the composite-dentin bond failed. S_{D}=4P/(πd²), where P is the load at failure, and d is the diameter of the composite (Zhang et al., 2012; Antonucci et al., 2009). Ten teeth were tested for each group, requiring a total of 80 teeth.

### 2.4. Specimen Fabrication for Protein Adsorption and Biofilm Experiments

The dentin shear bond strength results showed that the 7.5% MPC+7.5% DMAHDM group and the 7.5% MPC+10% DMAHDM group had significantly lower bond strengths. Therefore, 7.5% DMAHDM and 10% DMAHDM mass fractions were not used in subsequent experiments. The following four groups were used for protein adsorption and biofilm tests:
(1) SBMP primer and adhesive control (termed “SBMP control”);
(2) SBMP primer+7.5% MPC, SBMP adhesive+7.5% MPC (“7.5% MPC”);
(3) SBMP primer+5% DMAHDM, SBMP adhesive+5% DMAHDM (“5% DMAHDM”);
(4) SBMP primer+7.5% MPC+5% DMAHDM, SBMP adhesive+7.5% MPC+5% DMAHDM (“7.5% MPC+5% DMAHDM”).

The cover of a sterile 96-well plate (Costar, Corning Inc., Corning, N.Y.) was used for fabricating disk specimens following previous studies (Imazato et al., 2003a; Zhou et al., 2013a). Briefly, 10 μL of a primer was placed in the bottom of each dent. After drying with a stream of air, 20 μL of adhesive was applied to the dent and photo-polymerized for 30 s, using a mylar strip covering to obtain a disk of approximately 8 mm in diameter and 0.5 mm in thickness. The cured disks were immersed in 200 mL of distilled water and magnetically-stirred with a bar at a speed of 100 rpm for 1 h to remove any uncured monomers (Imazato et al., 1998; Cheng et al., 2012b). The disks were then sterilized with ethylene oxide (Anprolene AN 74i, Andersen, Haw River, N.C.) and de-gassed for 3 days (Zhou et al., 2013a).

### 2.5. Measurement of Protein Adsorption by Micro Bicinchoninic Acid Method

The amount of protein adsorbed on resin disks was determined by the micro bicinchoninic acid (BCA) method (Moro et al., 2009; Sibarani et al., 2007). Each disk was immersed in phosphate buffered saline (PBS) for 2 h. The disks then were immersed in bovine serum albumin (BSA) (Sigma-Aldrich) solutions at 37° C. for 2 h. The protein solutions contained BSA at a concentration of 4.5 g/L following previous studies (Moro et al., 2009; Sibarani et al., 2007). The disks then were rinsed with fresh PBS by stirring at a speed of 300 rpm for 5 min (Bellco Glass, Vineland, N.J.), immersed in sodium dodecyl sulfate (SDS) at 1 wt % in PBS, and sonicated at room temperature for 20 minutes to completely detach the BSA from disk surfaces. A protein analysis kit (micro BCA protein assay kit, Fisher Scientific, Pittsburgh, Pa.) was used to determine the BSA concentration in the SDS solution. From the concentration of protein, the amount of protein adsorbed on the resin disk was calculated (Moro et al., 2009; Sibarani et al., 2007). Six disks were evaluated for each group.

### 2.6. Saliva Collection for Biofilm Inoculum

The biofilm viability was investigated using a dental plaque microcosm model following previous studies (Cheng et al., 2012b; Zhang et al., 2012). Saliva is ideal for growing dental plaque microcosm biofilms in vitro, with the advantage of maintaining much of the complexity and heterogeneity of the dental plaque in vivo (McBain, 2009). Saliva was collected from ten healthy adult donors having natural dentition without active caries or periopathology, and without the use of antibiotics within the last 3 months, following previous studies (Cheng et al., 2012b; Zhang et al., 2012). The donors did not brush teeth for 24 hours and abstained from food and drink intake for 2 hours prior to donating saliva. Stimulated saliva was collected during parafilm chewing and was kept on ice. An equal volume of saliva from each of the ten donors was combined to form the saliva sample. The saliva was diluted in sterile glycerol to a concentration of 70%, and stored at −80° C. (Cheng et al., 2011).

### 2.7. Dental Plaque Microcosm Biofilm Formation and Live/Dead Assay

The saliva-glycerol stock was added, with 1:50 final dilution, to a growth medium as inoculum. The growth medium contained mucin (type II, porcine, gastric) at a concentration of 2.5 g/L; bacteriological peptone, 2.0 g/L; tryptone, 2.0 g/L; yeast extract, 1.0 g/L; NaCl, 0.35 g/L, KCl, 0.2 g/L; CaCl₂, 0.2 g/L; cysteine hydrochloride, 0.1 g/L; hemin, 0.001 g/L; vitamin K1, 0.0002 g/L, at pH 7 (McBain et al., 2005). 1.5 mL of inoculum was added to each well of 24-well plates containing a resin disk, and incubated at 37° C. in 5% CO₂ for 8 h. Then, the disks were transferred to new 24-well plates filled with fresh medium and incubated. After 16 h, the disks were transferred to new 24-well plates with fresh medium and incubated for 24 h. This totaled two days of culture, which formed microcosm biofilms as shown previously (Cheng et al., 2011).

Disks with 2-day biofilms were washed with PBS and stained using the BacLight live/dead kit (Molecular Probes, Eugene, Oreg.) (Cheng et al., 2012b; Zhang et al., 2012). Live bacteria were stained with Syto 9 to produce a green fluorescence, and bacteria with compromised membranes were stained with propidium iodide to produce a red fluorescence. The stained disks were examined using an inverted epifluorescence microscope (Eclipse TE2000-S, Nikon, Melville, N.Y.). Six specimens were evaluated for each group. Three randomly chosen fields of view were photographed for each disk, yielding a total of 18 images for each group.

### 2.8. Lactic Acid Production and Colony Forming Unit (CFU) Counts

Resin disks with 2-day biofilms were rinsed with cysteine peptone water (CPW) to remove loose bacteria (Cheng et al., 2012b). The disks were transferred to 24-well plates containing buffered peptone water (BPW) plus 0.2% sucrose. The disks were incubated in 5% CO₂ at 37° C. for 3 h to allow the biofilms to produce acid (Cheng et al., 2012b; Cheng et al., 2012a). The BPW solutions were then stored for lactate analysis. Lactate concentrations in the BPW solutions were determined using an enzymatic (lactate dehydrogenase) method, following a previous study (Cheng et al., 2012b). A microplate reader (SpectraMax M5, Molecular Devices, Sunnyvale, Calif.) was used to measure the absorbance at 340 nm (optical density OD340) for the collected BPW solutions. Standard curves were prepared using a lactic acid standard (Supelco, Bellefonte, Pa.) (Cheng et al., 2012b).

Disks with 2-day biofilms were transferred into tubes with 2 mL CPW, and the biofilms were harvested by sonication and vortexing (Fisher, Pittsburgh, Pa.) (Cheng et al., 2012b; Zhang et al., 2012). Three types of agar plates were used to measure the CFU counts to assess the microorganism viability. First, tryptic soy blood agar culture plates were used to determine total microorganisms (McBain et al., 2005). Second, mitis salivarius agar (MSA) culture plates containing 15% sucrose were used to determine total streptococci (Lima et al., 2009). Third, MSA agar culture plates plus 0.2 units of bacitracin per mL was used to determine mutans streptococci (McBain et al., 2005). The bacterial suspensions were serially diluted, spread onto agar plates and incubated at 37° C. in 5% CO₂ for 24 h (Cheng et al., 2012b; Zhang et al., 2012). The number of colonies that grew were counted and used, along with the dilution factor, to calculate the CFU on each resin disk (Cheng et al., 2012b; Zhang et al., 2012).

### 2.9. MTT Assay of Metabolic Activity of Biofilms

A MTT (3-[4,5-dimethylthiazol-2-yl]-2,5-diphenyltetrazolium bromide) assay was used to examine the metabolic activity of biofilms (Cheng et al., 2012b; Zhang et al., 2012). MTT is a colorimetric assay that measures the enzymatic reduction of MTT, a yellow tetrazole, to formazan. Disks with 2-day biofilms were transferred to a new 24-well plate, then 1 mL of MTT dye (0.5 mg/mL MTT in PBS) was added to each well and incubated at 37° C. in 5% CO₂ for 1 h. During this process, metabolically active bacteria reduced the MTT to purple formazan. After 1 h, the disks were transferred to a new 24-well plate, 1 mL of dimethyl sulfoxide (DMSO) was added to solubilize the formazan crystals, and the plate was incubated for 20 min at room temperature in the dark. After mixing via pipetting, 200 mL of the DMSO solution from each well was transferred to a 96-well plate, and the absorbance at 540 nm was measured via the microplate reader (SpectraMax M5) (Cheng et al., 2012b; Zhang et al., 2012). A higher absorbance is related to a higher formazan concentration, which indicates a higher metabolic activity in the biofilm on the disk (Cheng et al., 2012b; Zhang et al., 2012).

### 2.10. Statistical Analysis

One-way and two-way analyses of variance (ANOVA) were performed to detect the significant effects of the variables. Tukey's multiple comparison test was used to compare the data at a p value of 0.05.

### Results

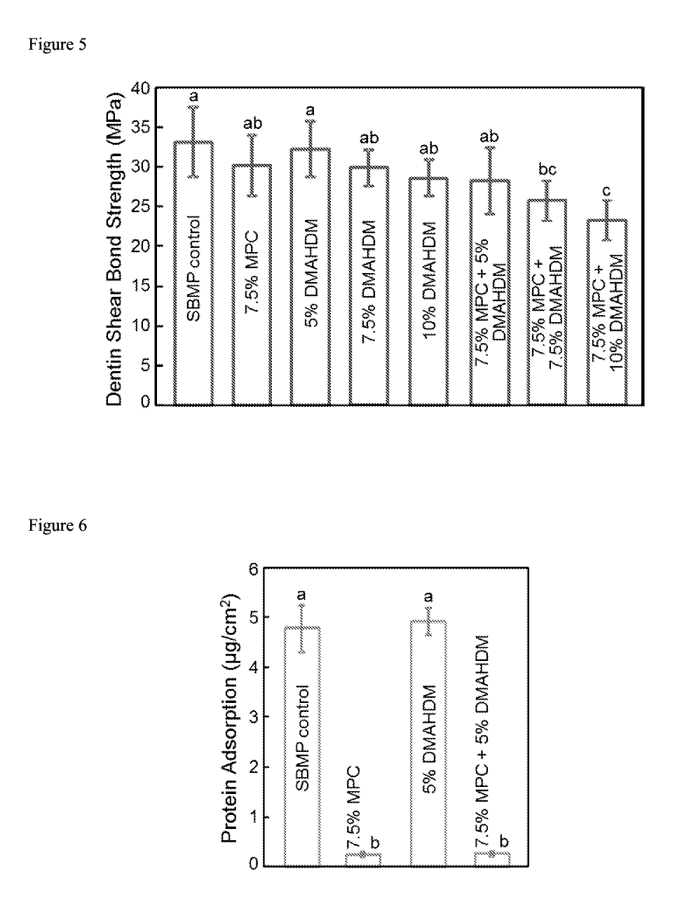

FIG. 5 plots the dentin shear bond results (mean±sd; n=10). The first six groups had similar dentin bond strengths (p>0.1). Incorporation of 7.5% MPC+5% DMAHDM into both primer and adhesive did not adversely affect the dentin shear bond strength, compared to SBMP control (p>0.1). However, the last two groups (7.5% MPC+7.5% DMAHDM) and (7.5% MPC+10% DMAHDM) had shear bond strengths that were significantly lower than SBMP control (p<0.05).

FIG. 6 plots the amount of protein adsorption on the resin surfaces (mean±sd; n=6). Adding 7.5% of MPC greatly reduced the protein adsorption, compared to SBMP control (p<0.05). Adding 5% DMAHDM had no effect on protein adsorption, compared to SBMP control. The 7.5% MPC+5% DMAHDM resin had the same protein adsorption as that of the resin with 7.5% MPC without DMAHDM (p>0.1). Therefore, DMAHDM had no effect on protein adsorption. The 7.5% MPC+5% DMAHDM resin had protein adsorption about 20-fold less than that of SBMP control (p<0.05).

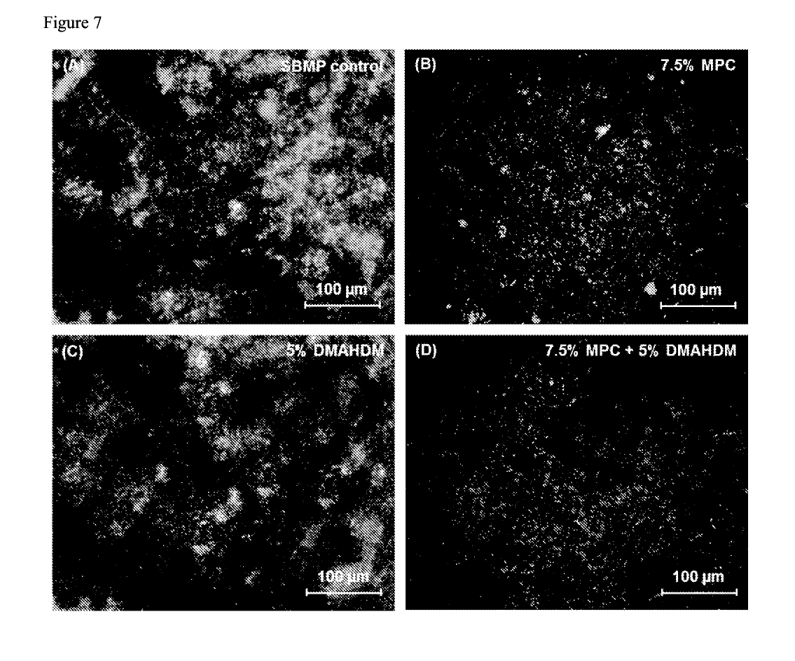

Typical live/dead staining images of 2-day biofilms grown on resin disks are shown in FIG. 7: (A) SBMP control, (B) 7.5% MPC, (C) 5% DMAHDM, (D) 7.5% MPC+5% DMAHDM. SBMP control was fully covered by a layer of biofilm consisting of primarily live bacteria. The resin with 7.5% MPC had much less bacterial adhesion. The resin with 5% DMAHDM had substantial amounts of dead bacteria with red staining. The resin with 7.5% MPC+5% DMAHDM had less bacterial adhesion, and the biofilms consisted of primarily dead bacteria with red staining.

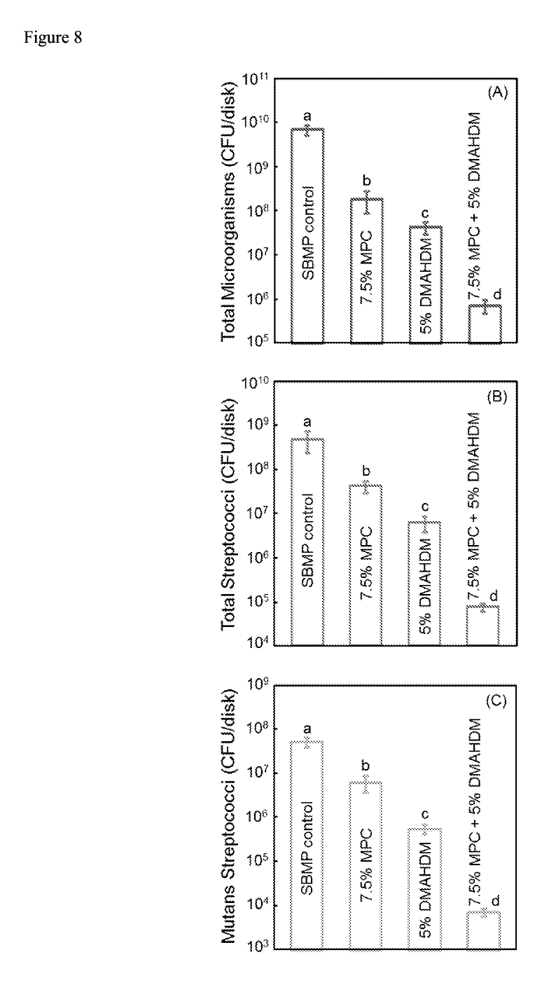

FIG. 8 plots the 2-day biofilm CFU counts for: (A) total microorganisms, (B) total streptococci, and (C) mutans streptococci (mean±sd; n=6). The values are shown in a log scale. Adding MPC or DMAHDM each decreased the biofilm CFU, compared to the SBMP control (p<0.05). The 7.5% MPC+5% DMAHDM resin had a much stronger antibacterial effect than using MPC or DMAHDM alone (p<0.05). All three CFU counts on the resin with 7.5% MPC+5% DMAHDM were more than 4 orders of magnitude lower than those on SBMP control.

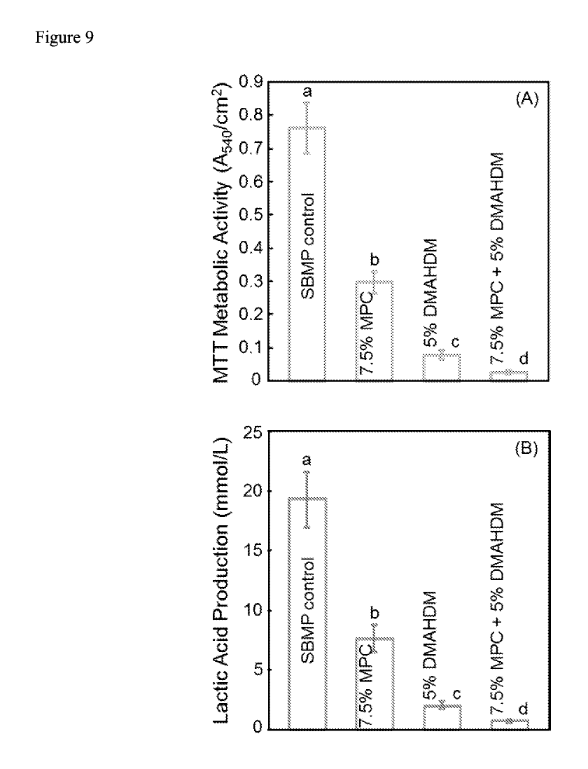

The metabolic activity and lactic acid production of 2-day biofilms on the resin disks are plotted in FIG. 9 (mean±sd; n=6). The biofilms on SBMP control had the highest metabolic activity and the most lactic acid production. Incorporation of MPC or DMAHDM each greatly decreased the metabolic activity and lactic acid production of the biofilms, compared to SBMP control (p<0.05). Using MPC and DMAHDM together in the resin resulted in the strongest antibacterial effect and the least biofilm activity. Biofilms on the resin containing 7.5% MPC+5% DMAHDM had the least metabolic activity and the least lactic acid production (p<0.05).

The results demonstrate that incorporating both MPC and an antibacterial agent into the resin results in both reduced protein adsorption and increased antibiotic activity over control resins. The incorporation of 7.5% MPC and 5% DMAHDM into the primer and adhesive yielded 20-fold less protein adsorption than SBMP control. There was a synergistic effect of MPC and DMAHDM in the adhesive on anti-biofilm properties. All three CFU counts on resins with 7.5% MPC or 5% DMAHDM alone were one or two orders of magnitude lower than that of SBMP control. However, when MPC and DMAHDM were both added into the bonding agent, the biofilms CFU counts were reduced by more than four orders of magnitude, compared to SBMP control. These results demonstrated the dramatically enhanced antibacterial efficacy when double agents (protein-repellant MPC+antibacterial DMAHDM) were used in the same adhesive system.

Regarding long-term durability, in the present study the SBMP primer and adhesive contained HEMA and copolymer of acrylic and itaconic acids, which could copolymerize with MPC when the adhesive was light-cured. Therefore, the MPC protein-repellant activity is expected to be durable.

The results demonstrate that dental adhesives with a combination of protein-repellent and antibacterial capabilities can be produced. In particular, these results demonstrated that: (1) a resin containing 7.5% MPC+5% DMAHDM had protein adsorption that was nearly 20-fold less than that of SBMP control; (2) adding MPC or DMAHDM each alone greatly decreased the biofilm viability, CFU and lactic acid production, compared to SBMP control; (3) adding both MPC and DMAHDM together in the same adhesive achieved antibacterial effects much stronger than MPC or DMAHDM alone, reducing biofilm CFU by four orders of magnitude; (4) incorporation of 7.5% MPC and 5% DMAHDM into primer and adhesive to obtain protein-repellent and antibacterial activities did not adversely affect the dentin shear bond strength. The novel protein-repellent and antibacterial adhesive is promising to inhibit biofilm acids and recurrent caries at the tooth-restoration margins. The method of using double agents (protein-repellent MPC+antibacterial DMAHDM) may be applicable in a wide range of dental adhesives, cements, sealants and composites to inhibit biofilms and caries.

### Example 3: Novel Protein-Repellent Dental Composite Containing 2-Methacryloyloxyethyl Phosphorylcholine

### Materials and Methods

### 3.1 Fabrication of MPC-Containing Resin Composites

BisGMA (bisphenol glycidyl dimethacrylate) and TEGDMA (triethylene glycol dimethacrylate) (Esstech, Essington, Pa.) were mixed at a mass ratio=1:1, and rendered light-curable with 0.2% camphorquinone and 0.8% ethyl 4-N,Ndimethylaminobenzoate (mass fractions). MPC (Sigma-Aldrich, St. Louis, Mo.) was commercially available, which was synthesized via a method reported by Ishihara et al., 1990. The MPC powder was mixed with the photo-activated BisGMA-TEGDMA resin (referred to as BT) at the following MPC/(BT+MPC) mass fractions: 0%, 2.5%, 5%, 7.5%, 10%, 15%, and 20%, yielding seven groups, respectively. The different mass fractions enabled the investigation of the relationship between MPC mass fraction and mechanical properties of the composite. A barium boroaluminosilicate glass of a mean particle size of 1.4 μm (Caulk/Dentsply, Milford, Del.) was silanized with 4% 3-methacryloxypropyltrimethoxysilane and 2% n-propylamine (Xu et al., 2011). The glass particles were mixed into each resin, at the same filler level of 70% by mass. Because the resin mass fraction was 30% in the composite, the MPC mass fractions in the composite were: 0% (control), 0.75%, 1.5%, 2.25%, 3.0%, 4.5%, and 6.0%, respectively. As another control, a commercial composite with nano-fillers of 40-200 nm was used (Heliomolar, Ivoclar, Ontario, Canada). The fillers consisted of silica and ytterbium-trifluoride at a filler mass fraction of 66.7%. Heliomolar is indicated for Class I and Class II restorations in the posterior region as well as Classes III-V restorations.

Eight composites were tested for mechanical properties:

(1) Commercial control (Heliomolar);
(2) 70% glass filler+30% BisGMA-TEGDMA resin (termed “Control with 0% MPC”);
(3) 70% glass filler+29.25% BisGMA-TEGDMA resin+0.75% MPC (“0.75% MPC”);
(4) 70% glass filler+28.5% BisGMA-TEGDMA resin+1.5% MPC (“1.5% MPC”);
(5) 70% glass filler+27.75% BisGMA-TEGDMA resin+2.25% MPC (“2.25% MPC”);
(6) 70% glass filler+27% BisGMA-TEGDMA resin+3% MPC (“3% MPC”);
(7) 70% glass filler+25.5% BisGMA-TEGDMA resin+4.5% MPC (“4.5% MPC”);
(8) 70% glass filler+24% BisGMA-TEGDMA resin+6% MPC (“6% MPC”).

### 3.2 Mechanical Properties

Each composite paste was placed into rectangular molds of 2 mm×2 mm×25 mm and photo-cured (Triad 2000, Dentsply, York, Pa.) for 1 min on each open side (Cheng et al., 2012a; Cheng et al., 2012d). Six specimens per composite were made. A computer-controlled Universal Testing Machine (5500R, MTS, Cary, N.C.) was used (Cheng et al., 2012a; Cheng et al., 2012d. Composite specimens were stored in distilled water at 37° C. for 24 h, and then fractured in three-point flexure with a 10 mm span at a crosshead speed of 1 mm/min (Cheng et al., 2012a; Cheng et al., 2012d). The specimens were wet and not dried, and were fractured within a few minutes after being taken out of the water. Flexural strength (S) was calculated as: S=3PₘₐₓL/(2bh²), where P is the fracture load, L is span, b is specimen width and h is thickness. Elastic modulus (E) was calculated as: E=(P/d)(L³/[4bh³]), where load P divided by displacement d is the slope in the linear elastic region (Cheng et al., 2012a; Cheng et al., 2012d).

### 3.3 Measurement of Protein Adsorption

The mechanical results showed that group 7 with 4.5% MPC and group 8 with 6% MPC had relatively lower strength and elastic modulus. Therefore, only groups 1-6 were included in further protein and biofilm experiments. For protein adsorption and biofilm experiments, each composite paste was placed into disk molds of 9 mm in diameter and 2 mm in thickness. They were light-cured and stored in distilled water at 37° C. for 24 h as described above.

The amount of protein adsorbed on composite disks was determined by the micro bicinchoninic acid (BCA) method (Ishihara et al., 1991; Moro et al., 2009). Each composite disk was immersed in phosphate buffered saline (PBS) for 2 h before immersing in 4.5 g/L bovine serum albumin (BSA) (Sigma-Aldrich) solutions at 37° C. for 2 h. The disks then were rinsed with fresh PBS by stirring at a speed of 300 rpm for 5 min (Bellco Glass, Vineland, N.J.), immersed in sodium dodecyl sulfate (SDS) 1 wt % in PBS, and sonication at room temperature for 20 minutes to completely detach the BSA adsorbed onto the surface of disk (Ishihara et al., 1991; Moro et al., 2009). A protein analysis kit (micro BCA protein assay kit, Fisher Scientific, Pittsburgh, Pa.) was used to determine the BSA concentration in the SDS solution. Briefly, mixing 25 μL of the SDS solution with 2004, of the BCA working reagent into a 96-well plate and incubated at 60° C. for 30 minutes (Ishihara et al., 1991; Moro et al., 2009). Then, the 96-well plate was cooled down to room temperature and the absorbance at 562 nm was measured via a microplate reader (SpectraMax M5, Molecular Devices, Sunnyvale, Calif.). Standard curves were prepared using BSA standard. From the concentration of protein, the amount of protein adsorbed on the disk surface was calculated (Ishihara et al., 1991 (Moro et al., 2009).

### 3.4 Dental Plaque Microcosm Biofilm Model

A dental plaque microcosm biofilm model was used (Cheng et al., 2012a; Cheng et al., 2012d). Saliva is ideal for growing microcosm biofilms in vitro, with the advantage of maintaining much of the complexity and heterogeneity of the dental plaque in vivo (McBain, 2005). Saliva was collected from ten healthy donors having natural dentition without active caries, and not having used antibiotics within the preceding 3 months. The donors did not brush teeth for 24 h and abstained from food and drink intake for 2 h prior to donating saliva (Cheng et al., 2012a; Cheng et al., 2012d). Stimulated saliva was collected during parafilm chewing and was kept on ice. An equal volume of saliva from each of the ten donors was combined to form the saliva sample. The saliva was diluted in sterile glycerol to a concentration of 70%, and stored at −80° C. for subsequent use (Cheng et al., 2011).

The saliva-glycerol stock was added, with 1:50 final dilution, into the growth medium as inoculum. The growth medium contained mucin (type II, porcine, gastric) at a concentration of 2.5 g/L; bacteriological peptone, 2.0 g/L; tryptone, 2.0 g/L; yeast extract, 1.0 g/L; NaCl, 0.35 g/L, KCl, 0.2 g/L; CaCl₂, 0.2 g/L; cysteine hydrochloride, 0.1 g/L; hemin, 0.001 g/L; vitamin K1, 0.0002 g/L, at pH 7 (McBain et al., 2005). Composite disks were sterilized in ethylene oxide (Anprolene AN 74i, Andersen, Haw River, N.C.). 1.5 mL of inoculum was added to each well of 24-well plates with a disk, and incubated at 37° C. in 5% CO₂ for 8 h. Then, the disks were transferred to new 24-well plates filled with fresh medium and incubated. After 16 h, the disks were transferred to new 24-well plates with fresh medium and incubated for 24 h. This totals 48 h of incubation, which was adequate to form plaque microcosm biofilms as shown in previous studies (Cheng et al., 2012a; Cheng et al., 2012d).

### 3.5 Live/Dead Assay

Two live/dead staining experiments were performed. First, the saliva-glycerol stock was added, with 1:50 final dilution, into the growth medium as inoculum. 1.5 mL of inoculum was added to each well of 24-well plates containing a composite disk, and incubated for 4 h to examine bacteria early-attachment (Zhou et al., 2013a). Second, using a separate batch of composite disks, the culture lasted for 48 h as described in Section 2.4.

After either 4 h and 48 h growth, the microcosm biofilms on composite disks were gently washed three times with phosphate buffered saline (PBS), and stained using the BacLight live/dead kit (Molecular Probes, Eugene, Oreg.). Live bacteria were stained with Syto 9 to produce a green fluorescence. Bacteria with compromised membranes were stained with propidium iodide to produce a red fluorescence. The stained disks were examined using an inverted epifluorescence microscope (Eclipse TE2000-S, Nikon, Melville, N.Y.). The area of green staining (live bacteria) was computed with NIS Elements imaging software (Nikon). The area fraction of live bacteria=green staining area/total area of the image. Six composite disks were evaluated for each group at each time period. Three randomly chosen fields of view were photographed from each disk, yielding a total of 18 images for each condition.

### 3.6 Biofilm Colony-Forming Unit (CFU) Counts

Disks with 48 h biofilms were transferred into tubes with 2 mL CPW, and the biofilms were harvested by sonication and vortexing (Fisher, Pittsburgh, Pa.) (Cheng et al., 2012a; Cheng et al., 2012d). Three types of agar plates were used to measure the CFU counts to assess the microorganism viability. First, tryptic soy blood agar culture plates were used to determine total microorganisms (McBain et al., 2005). Second, mitis salivarius agar (MSA) culture plates containing 15% sucrose were used to determine total streptococci (Lima et al., 2009). This is because MSA contains selective agents including crystal violet, potassium tellurite and trypan blue, which inhibit most Gram-negative bacilli and most Gram-positive bacteria except streptococci, thus enabling the streptococci to grow (Lima et al., 2009). Third, cariogenic mutans streptococci is known to be resistant to bacitracin, and this property is used to isolate mutans streptococci from the highly heterogeneous oral microflora (McBain et al., 2005). Therefore, MSA agar culture plates plus 0.2 units of bacitracin per mL was used to determine mutans streptococci (McBain et al., 2005). The bacterial suspensions were serially diluted, spread onto agar plates and incubated at 37° C. in 5% CO₂ for 24 h (Cheng et al., 2012a; Cheng et al., 2012d). The number of colonies that grew were counted and used, along with the dilution factor, to calculate total CFUs on each disk (Cheng et al., 2012a; Cheng et al., 2012d).

### 3.7 Statistical Analysis

One-way and two-way analyses of variance (ANOVA) were performed to detect the significant effects of the variables. Tukey's multiple comparison test was used to compare the data at a P value of 0.05.

### Results

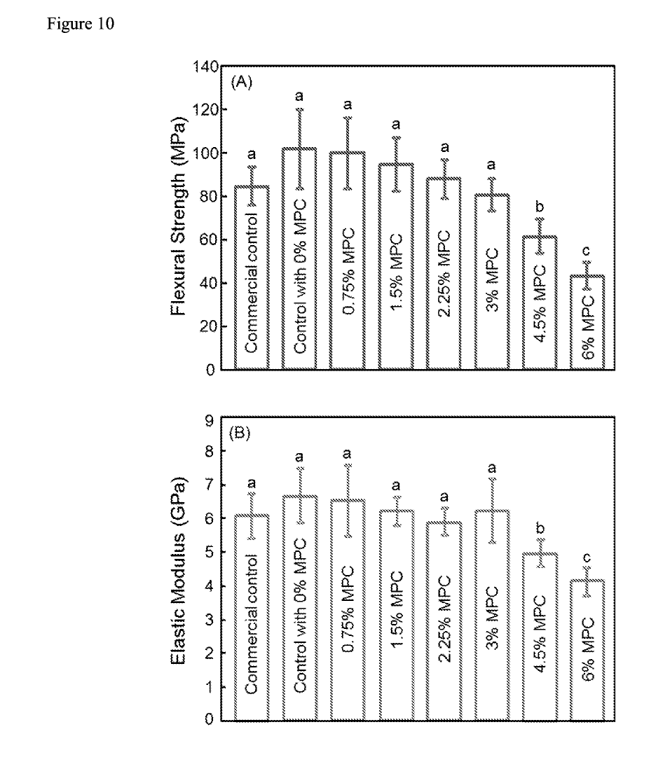

FIG. 10 plots (A) flexural strength, and (B) elastic modulus of the composites (mean±s.d.; n=6). The composites with 0.75% to 3% MPC had flexural strengths and elastic moduli similar to those of the commercial control and the control with 0% MPC (P>0.1). Composites with 4.5% MPC and 6% MPC had strengths and moduli that were significantly lower than those of commercial control (P<0.05).

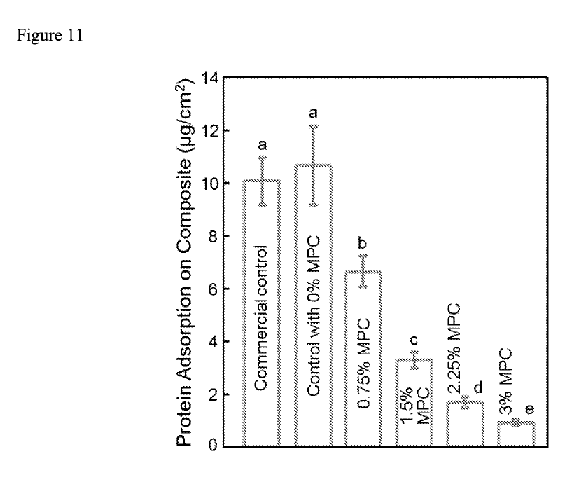

The amounts of protein adsorption on composite disk surfaces are plotted in FIG. 11 (mean±s.d.; n=6). Adding MPC into composite significantly decreased the protein adsorption (P<0.05). Protein adsorption was inversely proportional to the MPC mass fraction in the composite from 0% to 3% MPC. The resin composite with 3% MPC had the lowest amount of protein adsorption, which was nearly 1/12 that of commercial control and the composite with 0% MPC (P<0.05).

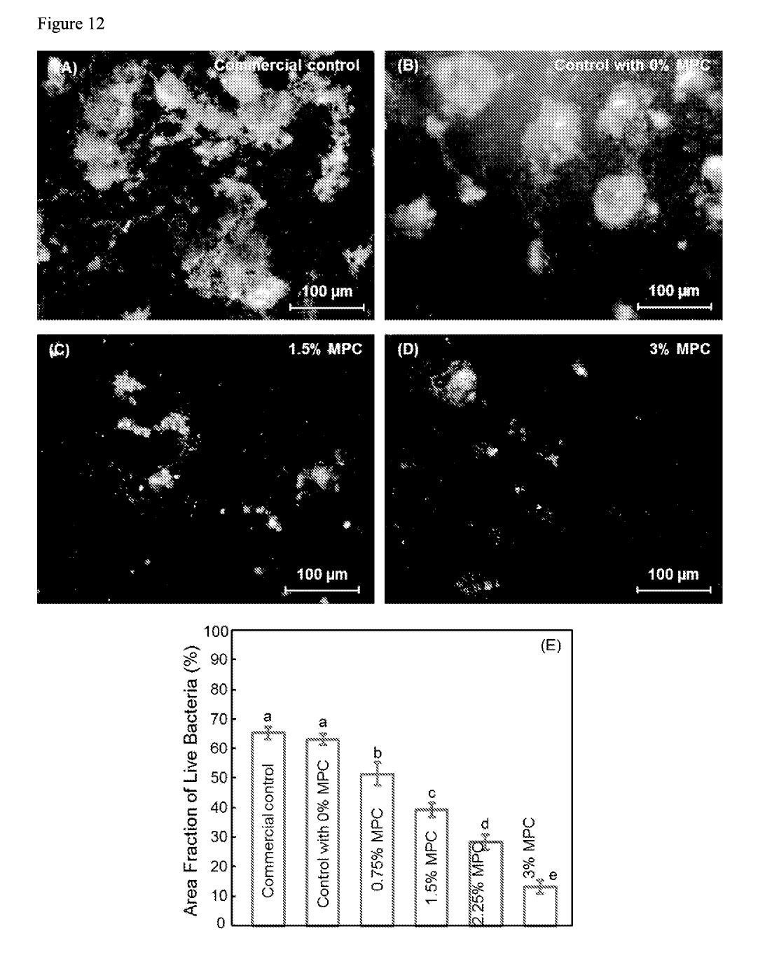

Bacterial early-attachment onto composite surfaces was examined at 4 h. Representative live/dead staining images at 4 h are shown in FIG. 12 (A-D), and the area fraction of composite surface covered by live bacteria is plotted in (E) (mean±s.d.; n=6). Live bacteria were stained green, and dead bacteria were stained red. The composite disks had primarily live bacteria, with few dead bacteria. The commercial control composite and the composite with 0% MPC had noticeably more bacteria coverage than composites containing MPC. This was true and consistent by examining all 18 images per group. The composite with 3% MPC had much less bacterial adhesion. In (E) for quantification of live bacteria coverage, values with dissimilar letters are significantly different from each other.

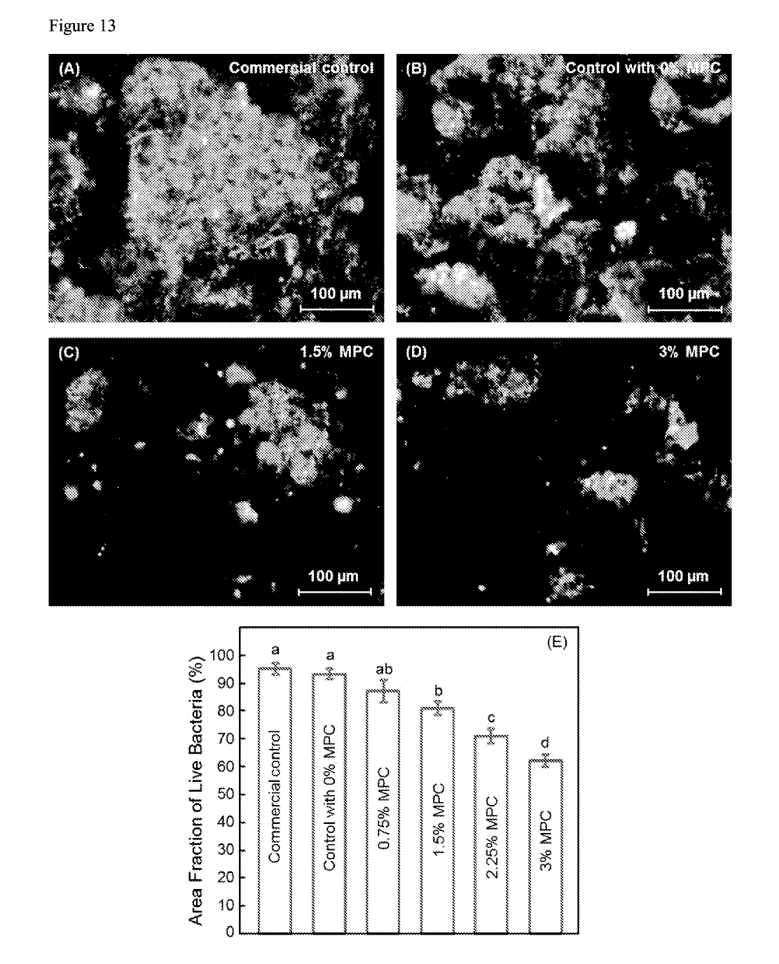

FIG. 13 shows results on 2-day biofilms on the composites. Relatively mature biofilms were formed in 2 days covering nearly the entire surface of commercial composite and that with 0% MPC (A and B). However, there was less biofilm coverage on composite disks containing MPC (C and D). In the quantification of the area fraction of live bacteria in (E) (mean±s.d.; n=6), composites with 1.5% to 3% MPC had significantly less biofilm coverage than the controls (P<0.05).

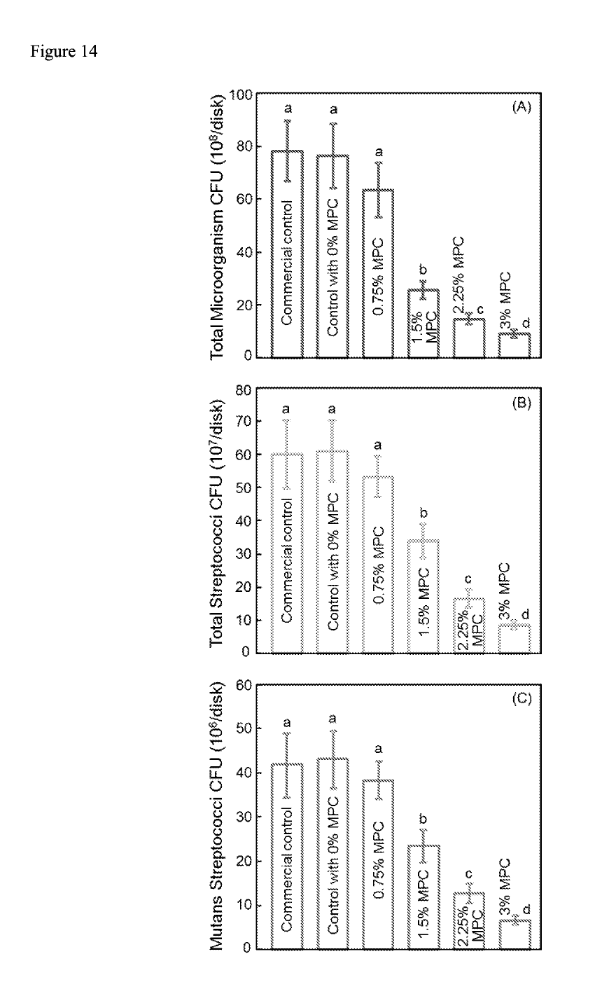

FIG. 14 plots the CFU counts of biofilms grown for 2 d on composite disks: (A) total microorganisms, (B) total streptococci, and (C) mutans streptococci (mean±s.d.; n=6). The commercial control composite and the experimental composite with 0% MPC had similarly high CFU counts (P>0.1). All three CFU counts showed a decreasing trend with increasing the MPC mass fraction. All three CFU counts on the composite with 3% MPC were greatly reduced, compared to those of the controls (P<0.05).

The results demonstrate that protein-repellent dental composite can be produced that have a significant effect on protein adsorption and biofilm activity, without affecting mechanical properties of the composite. MPC addition provided a strong protein-repellent activity for the composite, and the microcosm bacteria early-attachment (4 h) and mature biofilm (2 d) viability showed consistent and substantial decreases with increasing MPC mass fraction. The protein-repellent activity of the composite was achieved without compromising the mechanical properties of the composite from 0% to 3% of MPC.

Increasing the mass fraction of MPC in the resin composite increases the presence of MPC and hence its protein-repellent potency. Indeed, gradually increasing the MPC mass fraction from 0% to 3% in the composite significantly and monotonically decreased the amount of protein adsorption (FIG. 11). The fact that the MPC-containing composite can repel proteins would indicate that the composite could potentially also reduce biofilm attachment. Indeed, the results in FIGS. 12-14 confirmed that the incorporation of MPC at mass fraction of 3% into composite greatly reduced bacteria attachment, biofilm growth, and CFU counts.

The present study used an artificial saliva-like culture medium (the McBain medium) for the biofilm culture experiments. A major component of this medium, mucin, accounts for up to 26% of the natural salivary proteins; mucin is an important salivary protein in the salivary pellicle (Lendenmann et al., 2000; Amerongen et al., 1995). A recent study investigated the effects of salivary pellicle pre-coating on resin surfaces on the antibacterial properties of the resin (Li et al., 2014). When cultured in McBain medium, there was no significant difference in the antibacterial activity whether the resin specimens were pre-coated with salivary pellicles or not. These results suggested that the McBain artificial saliva medium produced medium-derived pellicles on the resin surfaces, which provided attenuating effects on biofilms similar to natural salivary pellicles. Hence, the present study used the McBain artificial saliva medium to test the bacteria attachment and biofilm growth on the MPC-containing composite, which better simulated the oral environment than using a culture medium like water.

Besides secondary caries due to biofilm acids, bulk fracture is also a main challenge facing composite restorations (Sakaguchi et al., 2005; Sarrett et al., 2005). Therefore, the new protein-repellent composite needs to possess load-bearing capability for tooth cavity restorations. Incorporating MPC into the composite at 0.75% to 3% MPC did not significantly decrease the composite strength. The strengths and elastic moduli of the MPC-containing resin composites were similar to those of a commercial composite used for Classes I to IV restorations. The strength and elastic modulus of the composite containing 3% MPC were also similar to those of the glass-filled composite without MPC. This indicates that significant protein-repellent capability can be achieved in dental composite without compromising the load-bearing capability, compared to the counterpart composite without MPC.

### Example 4: Protein-Repellent and Antibacterial Dental Composite to Inhibit Biofilms and Caries

### Materials and Methods

### 4.1. Preparation of Composites Containing MPC and DMAHDM

MPC was obtained commercially (Sigma-Aldrich, St. Louis, Mo.) which was synthesized via a method reported by Ishihara et al., 1990. BisGMA (bisphenol glycidyl dimethacrylate) and TEGDMA (triethylene glycol dimethacrylate) (Esstech, Essington, Pa.) were mixed at a mass ratio=1:1, and rendered light-curable with 0.2% camphorquinone and 0.8% ethyl 4-N,Ndimethylaminobenzoate (mass fractions). The MPC powder was mixed with the photo-activated BisGMA-TEGDMA resin (referred to as BT) at a MPC/(BT+MPC) mass fraction of 10%. Preliminary study on a series of mass fractions indicated that this mass fraction yielded a strong protein-repellent property without compromising mechanical properties of the resin.

Dimethylaminododecyl methacrylate (DMAHDM) with an alkyl chain length of 16 was synthesized using a modified Menschutkin reaction where a tertiary amine group was reacted with an organo-halide (Zhou et al., 2013a; Antonucci et al., 2012). A benefit of this reaction is that the reaction products are generated at virtually quantitative amounts and require minimal purification. Briefly, 10 mmol of 1-(dimethylamino)docecane (Sigma-Aldrich) and 10 mmol of 1-bromohexadecane (BHD, TCI America, Portland, Oreg.) were combined with 3 g of ethanol in a 20 mL scintillation vial. The vial was stirred at 70° C. for 24 h. The solvent was then removed via evaporation, yielding DMAHDM as a clear, colorless, and viscous liquid (Zhou et al., 2013b; Zhou et al., 2013a). DMAHDM was incorporated into the BisGMA-TEGDMA resin at DMAHDM/(BT+DMAHDM) mass fractions of 0%, 5%, 7.5%, and 10%. The 10% DMAHDM was used following previous studies (Zhou et al., 2013b; Zhou et al., 2013a). The 5% and 7.5% DMAHDM were used because 10% DMAHDM appeared to lower the composite strength when combined with MPC.

Each resin was filled with glass particles (barium boroaluminosilicate, mean size=1.4 Caulk/Dentsply, Milford, Del.) silanized with 4% 3-methacryloxypropyltrimethoxysilane and 2% n-propylamine. A filler mass fraction of 70% was used to yield a cohesive paste. Since the resin mass fraction in the composite was 30%, the MPC mass fraction in the final composite was 3%. The DMAHDM mass fractions in the composite were 0%, 1.5%, 2.25%, and 3%, respectively. The composite with 0% MPC and 0% DMAHDM served as a control. In addition, a commercial composite (Heliomolar, Ivoclar, Ontario, Canada) also served as a control. The fillers were silica and ytterbium-trifluoride with particle sizes of 40-200 nm at a filler level of 66.7%. Heliomolar is indicated for Class I and Class II restorations in the posterior region and Classes III-V restorations.

### 4.2. Mechanical Properties

Nine composites were tested for mechanical properties:
(1) Commercial control (Heliomolar);
(2) Experimental control: 70% glass+30% BT (termed “0% MPC+0% DMAHDM”);
(3) 70% glass+27% BT+3% MPC (termed “3% MPC”);
(4) 70% glass+28.5% BT+1.5% DMAHDM (“1.5% DMAHDM”);
(5) 70% glass+27.75% BT+2.25% DMAHDM (“2.25% DMAHDM”);
(6) 70% glass+27% BT+3% DMAHDM (“3% DMAHDM”);
(7) 70% glass+25.5% BT+3% MPC+1.5% DMAHDM (“3% MPC+1.5% DMAHDM”);
(8) 70% glass+24.75% BT+3% MPC+2.25% DMAHDM (“3% MPC+2.25% DMAHDM”);
(9) 70% glass+24% BT+3% MPC+3% DMAHDM (“3% MPC+3% DMAHDM”).

Each composite paste was placed into rectangular molds of 2 mm×2 mm×25 mm. The specimens were photo-cured (Triad 2000, Dentsply, York, Pa.) for 1 min on each open side (Cheng et al., 2012d; Cheng et al., 2012c. Six specimens were made for each composite. The specimens were immersed in distilled water at 37° C. for 24 h (Cheng et al., 2012d; Cheng et al., 2012c). The specimens were then fractured in three-point flexure with a 10-mm span at a crosshead-speed of 1 mm/min on a computer-controlled Universal Testing Machine (5500R, MTS, Cary, N.C.). Flexural strength (S) was calculated as: S=3PₘₐₓL/(2bh²), where P is the fracture load, L is span, b is specimen width and h is thickness. Elastic modulus (E) was calculated as: E=(P/d)(L³/[4bh³]), where load P divided by displacement d is the slope in the linear elastic region. The specimens were wet and not dried, and were fractured within a few minutes after being taken out of the water (Cheng et al., 2012d; Cheng et al., 2012c).

### 4.3. Characterization of Protein Adsorption

The mechanical testing results showed that composites with 3% MPC plus 2.25% or 3% DMAHDM had lower composite strength. Therefore, only the 1.5% DMAHDM was used. Hence, five composites were tested for protein adsorption and biofilm experiments:

(1) Commercial control (Heliomolar);
(2) Experimental control: 70% glass+30% BT (termed “0% MPC+0% DMAHDM”);
(3) 70% glass+27% BT+3% MPC (termed “3% MPC”);
(4) 70% glass+28.5% BT+1.5% DMAHDM (“1.5% DMAHDM”);
(5) 70% glass+25.5% BT+3% MPC+1.5% DMAHDM (“3% MPC+1.5% DMAHDM”).

For protein adsorption and biofilm experiments, each composite paste was placed into disk molds of 9 mm in diameter and 2 mm in thickness and light-curd as described above. The specimens were immersed in distilled water at 37° C. for 24 h. The amount of protein adsorbed on composite disks was determined by the micro bicinchoninic acid (BCA) method (Ishihara et al., 1991; Sibarani et al., 2007). Each disk was immersed in phosphate buffered saline (PBS) for 2 h. The disks then were immersed in bovine serum albumin (BSA) (Sigma-Aldrich) solutions at 37° C. for 2 h. The protein solutions contained BSA at a concentration of 4.5 g/L following previous studies (Ishihara et al., 199; Sibarani et al., 2007). The disks then were rinsed with fresh PBS by stirring at a speed of 300 rpm for 5 min (Bellco Glass, Vineland, N.J.), immersed in sodium dodecyl sulfate (SDS) 1 wt % in PBS, and sonication at room temperature for 20 minutes to completely detach the BSA adsorbed onto the surface of disk (Ishihara et al., 1991; Sibarani et al., 2007). A protein analysis kit (micro BCA protein assay kit, Fisher Scientific, Pittsburgh, Pa.) was used to determine the BSA concentration in the SDS solution (Ishihara et al., 1991; Sibarani et al., 2007). Briefly, 25 μL of the SDS solution was mixed with 200 μL of the BCA working reagent in a 96-well plate, which was incubated at 60° C. for 30 minutes (Ishihara et al., 1991; Sibarani et al., 2007). Then the 96-well plate was cooled down to room temperature and the absorbance at 562 nm was measured via a microplate reader (SpectraMax M5, Molecular Devices, Sunnyvale, Calif.). Standard curves were prepared using the BSA standard. From the concentration of protein, the amount of protein adsorbed on the composite disk surface was calculated (Ishihara et al., 1991; Sibarani et al., 2007).

### 4.4. Saliva Collection for Biofilm Inoculum

Oral biofilm growth and viability on the composite disks were investigated using a dental plaque microcosm model following previous studies (Cheng et al., 2012d; Cheng et al., 2012c). Saliva is ideal for growing dental plaque microcosm biofilms in vitro, with the advantage of maintaining much of the complexity and heterogeneity of the dental plaque in vivo (McBain, 2009). The saliva for biofilm inoculums was collected from ten healthy adult donors having natural dentition without active caries or periopathology, and without the use of antibiotics within the last 3 months, following previous studies (Cheng et al., 2012d; Cheng et al., 2012c). The donors did not brush teeth for 24 h and abstained from food and drink intake for 2 h prior to donating saliva. Stimulated saliva was collected during parafilm chewing and was kept on ice. An equal volume of saliva from each of the ten donors was combined to form the saliva sample. The saliva was diluted in sterile glycerol to a concentration of 70%, and stored at −80° C. (Cheng et al., 2011).

### 4.5. Dental Plaque Microcosm Biofilm Formation and Live/Dead Assay

The saliva-glycerol stock was added, with 1:50 final dilution, into the growth medium as inoculum (Cheng et al., 2012d; Cheng et al., 2012c). The growth medium contained mucin (type II, porcine, gastric) at a concentration of 2.5 g/L; bacteriological peptone, 2.0 g/L; tryptone, 2.0 g/L; yeast extract, 1.0 g/L; NaCl, 0.35 g/L, KCl, 0.2 g/L; CaCl₂, 0.2 g/L; cysteine hydrochloride, 0.1 g/L; hemin, 0.001 g/L; vitamin K1, 0.0002 g/L, at pH 7 (McBain et al., 2005). The composite disks were sterilized in ethylene oxide (Anprolene AN 74i, Andersen, Haw River, N.C.). 1.5 mL of inoculum was added to each well of 24-well plates with a disk, and incubated at 37° C. in 5% CO₂ for 8 h. Then, the composite disks were transferred to new 24-well plates filled with fresh medium and incubated. After 16 h, the composite disks were transferred to new 24-well plates with fresh medium and incubated for 24 h. This totaled 48 h of incubation, which was adequate to form plaque microcosm biofilms as shown in a previous study (Cheng et al., 2011).

For live/dead bacterial staining assay, composite disks with 2-day biofilms were washed with PBS and stained using the BacLight live/dead kit (Molecular Probes, Eugene, Oreg.) (Cheng et al., 2012d; Cheng et al., 2012c). Live bacteria were stained with Syto 9 to produce a green fluorescence. Bacteria with compromised membranes were stained with propidium iodide to produce a red fluorescence. The stained disks were examined using an inverted epifluorescence microscope (Eclipse TE2000-S, Nikon, Melville, N.Y.). The area of green staining (live bacteria) was computed with NIS Elements imaging software (Nikon). The area fraction of live bacteria=green staining area/total area of the image (Cheng et al., 2012c). Six specimens were evaluated for each composite. Three randomly chosen fields of view were photographed from each disk, yielding a total of 18 images for each composite.

### 4.6. MTT Metabolic Assay

Composite disks with 2-day biofilms were transferred to a new 24-well plate for the MTT [3-(4,5-Dimethylthiazol-2-yl)-2,5-diphenyltetrazolium bromide] assay (Cheng et al., 2012d; Cheng et al., 2012c). MTT is a colorimetric assay that measures the enzymatic reduction of MTT, a yellow tetrazole, to formazan. A total of 1 mL of MTT was added to each well and incubated for 1 h. Disks were transferred to a new 24-well plate, and 1 mL of dimethyl sulfoxide (DMSO) was added to solubilize the formazan crystals. The plate was incubated for 20 min with gentle mixing at room temperature in the dark. Then, 200 μL of the DMSO solution from each well was collected, and its absorbance at 540 nm was measured via a microplate reader (SpectraMax M5, Molecular Devices, Sunnyvale, Calif.). A higher absorbance is related to a higher formazan concentration, which indicates a higher metabolic activity in the biofilm on the disk (Cheng et al., 2012d; Cheng et al., 2012c).

### 4.7. Lactic Acid Production

Composite disks with 2-day biofilms were rinsed with cysteine peptone water (CPW) to remove loose bacteria (Cheng et al., 2012d; Cheng et al., 2012c). The disks were transferred to 24-well plates and 1.5 mL of buffered-peptone water (BPW) supplemented with 0.2% sucrose was added. The disks were incubated at 37° C. in 5% CO₂ for 3 h to allow the biofilms to produce acid (Cheng et al., 2012d; Cheng et al., 2012c). The BPW solutions were then stored for lactate analysis. Lactate concentrations in the BPW solutions were determined using an enzymatic (lactate dehydrogenase) method, following previous studies (Cheng et al., 2012d; Cheng et al., 2012c). The microplate reader was used to measure the absorbance at 340 nm (optical density OD340) for the collected BPW solutions. Standard curves were prepared using a lactic acid standard (Supelco, Bellefonte, Pa.) (Cheng et al., 2012d; Cheng et al., 2012c).

### 4.8. Colony Forming Unit (CFU) Counts

Composite disks with 2-day biofilms were transferred into tubes with 2 mL CPW, and the biofilms were harvested by sonication and vortexing (Fisher, Pittsburgh, Pa.) (Cheng et al., 2012d; Cheng et al., 2012c). Three types of agar plates were used to measure the CFU counts to assess the microorganism viability. First, tryptic soy blood agar culture plates were used to determine total microorganisms (McBain et al., 2005). Second, mitis salivarius agar (MSA) culture plates containing 15% sucrose were used to determine total streptococci (Lima et al., 2009). This is because MSA contains selective agents including crystal violet, potassium tellurite and trypan blue, which inhibit most Gram-negative bacilli and most Gram-positive bacteria except streptococci, thus enabling the streptococci to grow (Lima et al., 2009). Third, cariogenic mutans streptococci is known to be resistant to bacitracin, and this property is used to isolate mutans streptococci from the highly heterogeneous oral microflora (McBain et al., 2005). Therefore, MSA agar culture plates plus 0.2 units of bacitracin per mL was used to determine mutans streptococci (McBain et al., 2005). The bacterial suspensions were serially diluted, spread onto agar plates and incubated at 37° C. in 5% CO₂ for 24 h (Cheng et al., 2012d; Cheng et al., 2012c). The number of colonies that grew was counted and used, along with the dilution factor, to calculate the CFU counts on each composite disk (Cheng et al., 2012d; Cheng et al., 2012c).

### 4.9. Statistical Analysis

One-way and two-way analyses of variance (ANOVA) were performed to detect the significant effects of the variables. Tukey's multiple comparison test was used to compare the data at a p value of 0.05.

### Results

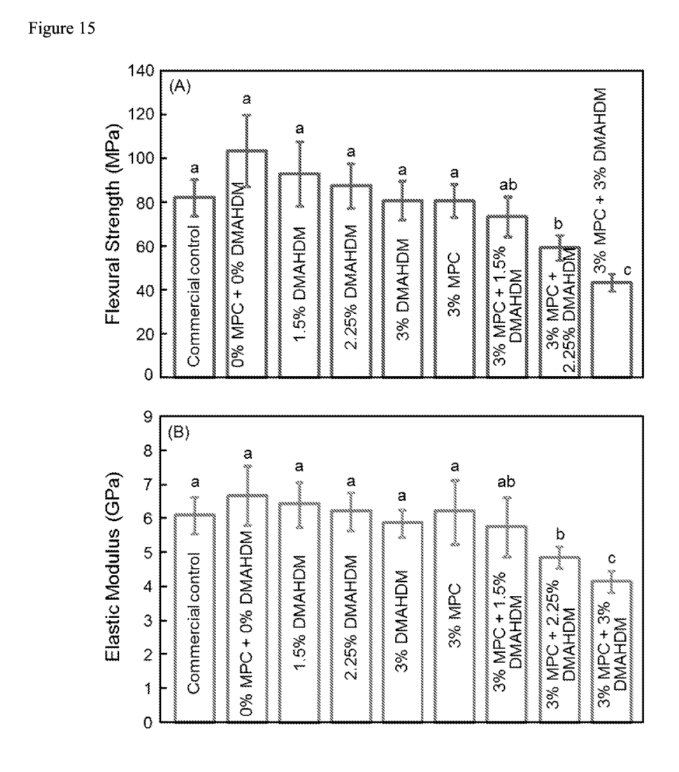

FIG. 15 plots of the mechanical properties of composites: (A) Flexural strength, and (B) elastic modulus (mean±sd; n=6). The first seven composites had flexural strength and elastic modulus that were not significantly different from each other (p>0.1). The last two composites with 3% MPC+2.25% DMAHDM and 3% MPC+3% DMAHDM had significantly lower mechanical properties than the controls (p<0.05). The composite containing 3% MPC+1.5% DMAHDM had strength and elastic modulus similar to those of the commercial control (p>0.1).

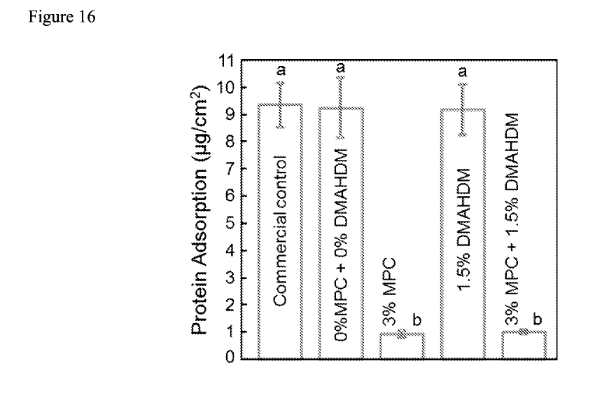

Protein adsorption onto composite surfaces is plotted in FIG. 16 (mean±sd; n=6). Adding 3% of MPC into the composite greatly reduced the protein adsorption, compared to that with 0% MPC and the commercial control (p<0.05). Adding 1.5% DMAHDM had no effect on protein adsorption (p>0.1). The composite with 3% MPC+1.5% DMAHDM had the same protein adsorption as that containing 3% MPC without DMAHDM (p>0.1). The composite with 3% MPC+1.5% DMAHDM had protein adsorption about an order of magnitude less than that of control (p<0.05).

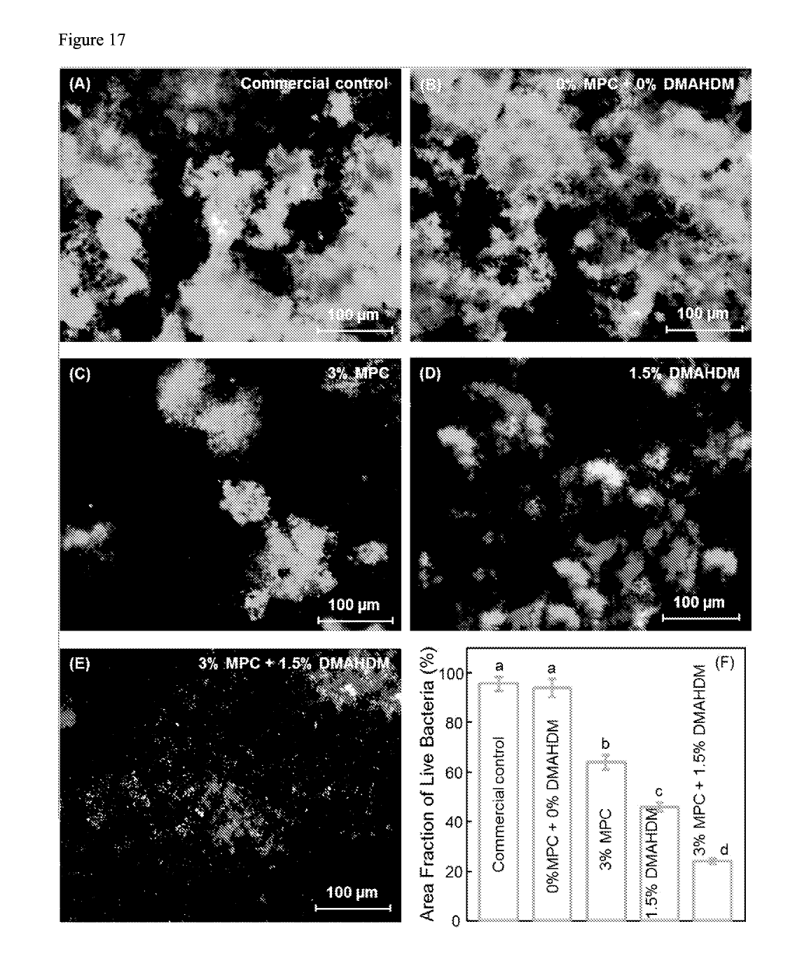

Typical live/dead staining images of 2-day biofilms grown on composite disks are shown in FIG. 17 (A-E), and the area fraction of composite surface covered by live bacteria is plotted in (F) (mean±sd; n=6). The two control composites were fully covered by live bacteria (A and B). In contrast in (C), composite with 3% MPC had much less bacterial adhesion, although the bacteria were mostly alive (green staining). On the other hand, composite with 1.5% DMAHDM had a strong antibacterial activity yielding substantial amounts of dead bacteria (red staining). The yellowish staining was likely caused by live and dead bacteria being close together or on the top of each other. Finally, the combined use of 3% MPC+1.5% DMAHDM had much less bacterial adhesion, and the bacteria were mostly dead. In (F) for quantification of live bacteria coverage, values with dissimilar letters are significantly different from each other.

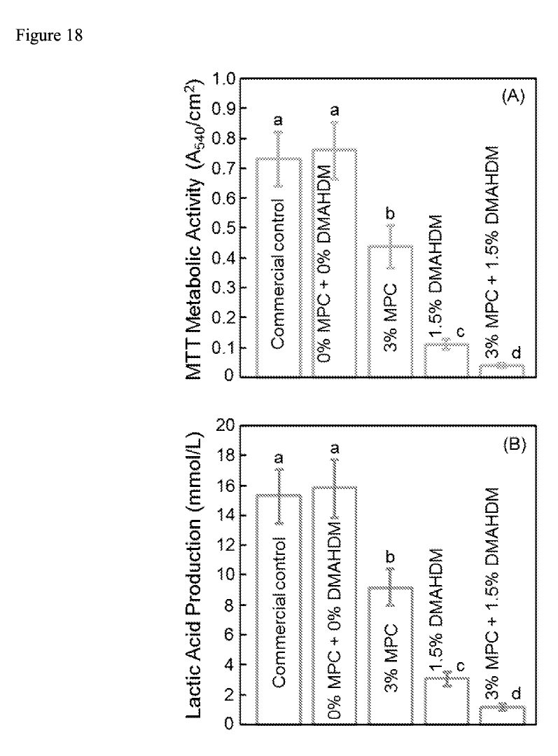

Quantitative viability of the 2-day biofilms on composites is shown in FIG. 18: (A) Metabolic activity, and (B) lactic acid production (mean±sd; n=6). Incorporation of MPC or DMAHDM alone greatly decreased the metabolic activity and lactic acid production of the biofilms, compared to the controls (p<0.05). The composite containing 3% MPC+1.5% DMAHDM had the least metabolic activity and lactic acid production.

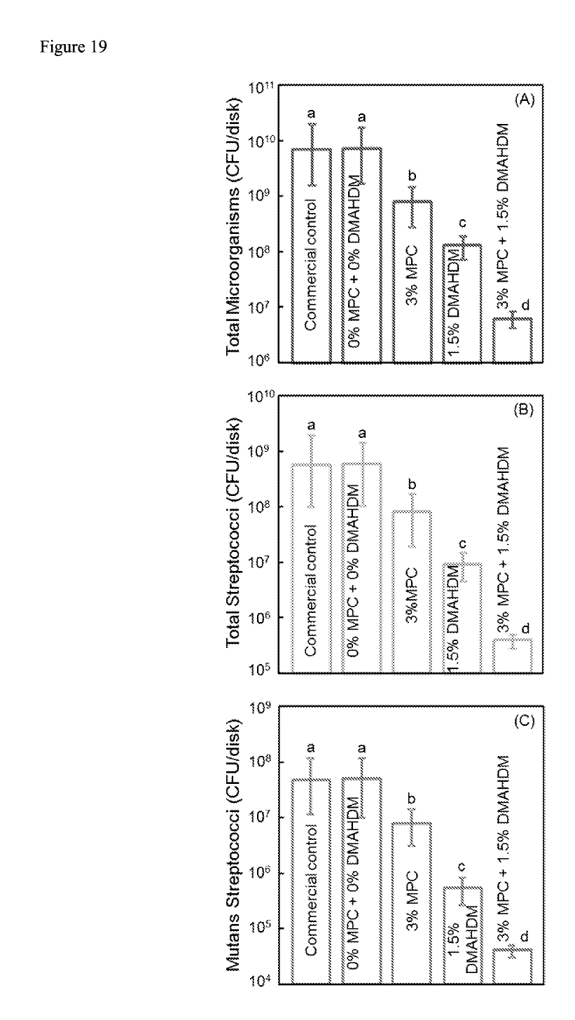

FIG. 19 plots the CFU of the 2-day biofilms grown on the composite disks: (A) Total microorganisms, (B) total streptococci, and (C) mutans streptococci (mean±sd; n=6). Incorporating MPC or DMAHDM alone into the composite decreased the biofilm CFU, compared to the two controls (p<0.05). The composite with double agents, 3% MPC+1.5% DMAHDM, had much less biofilm CFU than using either MPC or DMAHDM alone (p<0.05). All three CFU counts on the composite with 3% MPC+1.5% DMAHDM were more than 3 orders of magnitude lower than those of the two control composites.

In the present study, a novel composite was developed with MPC for protein-repellent ability and DMANDM for antibacterial property. The results demonstrate that the composite with 3% MPC+1.5% DMAHDM greatly reduced protein adsorption, bacteria attachment and biofilm growth, metabolic activity, CFU counts, and lactic acid production. These benefits were achieved without compromising mechanical properties. Therefore, the composite with 3% MPC+1.5% DMAHDM is promising for dental restorations to inhibit biofilm acids and secondary caries, and the approach of using dual agents with protein-repellent and antibacterial capabilities may have applicability to other dental materials.

The results of protein adsorption assay confirmed that incorporation of MPC into the composite significantly decreased protein adsorption. This was confirmed via the composite containing 3% MPC, which had protein adsorption 1/10 that of control composite. The present study also confirmed that the MPC composite with protein-repellent capability indeed had much less bacteria attachment and biofilm CFU.

QAMs have been shown to be promising for dental applications including use in composite, primer and adhesive (Imazato et al., 2003c; Imazato et al., 2009; Beyth et al., 2006; Namba et al., 2009; Li et al., 2009; Cheng et al., 2012a; Cheng et al., 2012d; Cheng et al., 2012c; Zhou et al., 2013b; He et al., 2011; Zhou et al., 2013a). In the present study, DMAHDM and MPC were combined for use in the composite. The results showed that DMAHDM indeed imparted a strong antibacterial function to the composite. Furthermore, the present study showed that the antibacterial potency of DMAHDM-containing composite can be further increased by the incorporation of MPC. The combined use of MPC and DMAHDM in the composite was supported by two benefits: (1) the use of dual agents in the composite achieved much greater reduction in biofilm activity, compared to DMAHDM or MPC alone; (2) the use of dual agents in the composite did not adversely affect the mechanical properties. All three CFU counts of biofilm on the composite with 1.5% DMAHDM were nearly two orders of magnitude lower than the control. However, the composite with 3% MPC+1.5% DMAHDM reduced the biofilm CFU by more than three orders of magnitude. The reason may be that, the mode of antibacterial action of DMAHDM-containing composite is contact-inhibition (Beyth et al., 2006; Namba et al., 2009). Previous studies suggested that when the negatively-charged bacterial cell contacts the positively-charged sites of QAM, the electric balance of the cell membrane could be disturbed, and the bacterium could explode under its own osmotic pressure (Beyth et al., 2006; Namba et al., 2009; Murata et al., 2007). This contact-killing mechanism would indicate that, when a salivary protein pellicle separates the antibacterial resin surface from the overlaying biofilm, the antibacterial effect of the resin could be decreased (Beyth et al., 2006; Namba et al., 2009). Indeed, several studies demonstrated that a saliva-derived protein film on the cationic antibacterial surfaces reduced the original bactericidal effect (Müller et al., 2009; Li et al., 2014; Imazato et al., 2003b). Because MPC can greatly reduce the protein adsorption, it would enhance the antibacterial effectiveness of DMAHDM. This factor likely contributed to the further reduction in biofilm CFU by more than an order of magnitude over that with DMAHDM alone.

Regarding the long-term durability of protein-repellent and antibacterial properties, the advantage of QAM composite is that the antibacterial agent is copolymerized with the resin by forming a covalent bonding with the polymer network (Imazato et al., 2003c; Imazato et al., 2009). Therefore the QAM is immobilized in the composite and not released or lost over time (Imazato et al., 2003c; Imazato et al., 2009). This method imparts a durable antibacterial capability to the composite. Regarding the MPC, in the present study MPC was mixed and copolymerized with the BisGMA-TEGDMA resin, leading to MPC immobilization in the composite. Therefore, the MPC was incorporated throughout the composite volume, and not limited to the surface only; as a result, the MPC effect will not be lost in wear and chewing actions. Therefore, the protein-repellent activity of the composite is expected to be durable.

Regarding potential clinical applications, the composite containing MPC and DMAHDM may be especially useful in patients who are prone to developing caries. Furthermore, the dual agents method of MPC plus DMAHDM may have a wide applicability to other types of dental materials. This includes bonding agents, cements, sealants and various types of composites such as flowable composites for root caries restorations. Studies are needed to incorporate MPC and DMAHDM into various dental materials to gain protein-repellent and antibacterial benefits without adversely affecting other desirable properties.

### Example 5: Development of a Multifunctional Adhesive Coating System for Prevention of Root Caries

### Materials and Methods

5.1. MPC Incorporation into Bonding Agent

Scotchbond multi-purpose (3M, St. Paul, Minn.), referred to as “SBMP”, was used as the parent bonding system to develop the novel MPC-DMAHDM-NACP containing adhesive coating system for prevention of root caries. The purpose was to investigate a model system, and then the method of incorporating MPC, DMAHDM and NACP could be applied to other bonding agents. According to the manufacturer, SBMP etchant contained 35% phosphoric acid. SBMP primer single bottle contained 35-45% 2-hydroxyethylmethacrylate (HEMA), 10-20% copolymer of acrylic and itaconic acids, and 40-50% water. SBMP adhesive contained 60-70% BisGMA and 30-40% HEMA.

MPC was obtained commercially (Sigma-Aldrich, St. Louis, Mo.) which was synthesized via a method reported by Ishihara et al., 1990. The MPC powder was mixed with SBMP primer at MPC/(SBMP primer+MPC) mass fraction of 7.5%. This mass fraction was selected from preliminary study showing that 7.5% MPC yielded the strongest protein-repellent property without compromising the dentin shear bond strength. MPC was added into primer and magnetically-stirred with a bar at a speed of 150 rpm (Bellco Glass, Vineland, N.J.) for 24 h to completely dissolve MPC in primer. Similarly, 7.5% of MPC was also mixed into the SBMP adhesive.

5.2. DMAHDM Incorporation into Bonding Agent

DMAHDM was synthesized using a modified Menschutkin reaction where a tertiary amine was reacted with an organo-halide (Zhou et al., 2013a). A benefit of this reaction is that the reaction products are generated at virtually quantitative amounts and require minimal purification. Briefly, 10 mmol of 1-(dimethylamino)docecane (Sigma-Aldrich) and 10 mmol of 1-bromohexadecane (BHD, TCI America, Portland, Oreg.) were combined with 3 g of ethanol in a 20 mL scintillation vial. The vial was stirred at 70° C. for 24 h. The solvent was then removed via evaporation, yielding DMAHDM as a clear, colorless, and viscous liquid (Zhou et al., 2013a). The SBMP primer was first mixed with MPC as described above. Then, DMAHDM was mixed into the SBMP-MPC primer, at DMAHDM/(SBMP primer+DMAHDM) mass fractions of 5%. DMAHDM mass fractions of 7.5% or higher were not used due to a decrease in dentin bond strength when combined with MPC in preliminary study. Similarly, 5% DMAHDM was incorporated into the SBMP-MPC adhesive.

5.3. NACP Incorporation into Bonding Agent

A spray-drying technique was used to synthesize NACP (Ca₃[PO₄]₂) as described previously (Xu et al., 2006; Xu et al., 2010). Briefly, calcium carbonate and dicalcium phosphate anhydrous were dissolved in acetic acid to produce Ca and P concentrations of 8 mmol/L and 5.333 mmol/L, respectively, thus yielding a Ca/P molar ratio=1.5, the same as that for ACP. This solution was sprayed into a heated chamber of the spray-drying apparatus. The dried particles were collected via an electrostatic precipitator (AirQuality, Minneapolis, Minn.). This yielded NACP with a mean particle size of 116 nm (Xu et al., 2006; Xu et al., 2010). NACP were incorporated into the SBMP adhesive, but not into the primer, because preliminary study showed that adding NACP into primer decreased the dentine bond strength (Zhang et al., 2013; Melo et al., 2013). NACP was mixed into the adhesive at NACP/(SBMP adhesive+NACP) mass fractions of 0%, 20%, 30%, and 40%, respectively. NACP mass fraction of less than 20% was not used due to the need for sufficient ion release, and NACP of more than 40% was not used due to a decrease in dentin bond strength (Zhang et al., 2013; Melo et al., 2013).

The following five groups were tested for dentin shear bond strength:
(1) SBMP primer and adhesive control (referred to as “SBMP control”).
(2) SBMP primer+7.5% MPC+5% DMAHDM. SBMP adhesive+7.5% MPC+5% DMAHDM (referred to as “7.5% MPC+5% DMAHDM”).
(3) SBMP primer+7.5% MPC+5% DMAHDM. SBMP adhesive+7.5% MPC+5% DMAHDM+20% NACP (“7.5% MPC+5% DMAHDM+20% NACP”).
(4) SBMP primer+7.5% MPC+5% DMAHDM. SBMP adhesive+7.5% MPC+5% DMAHDM+30% NACP (“7.5% MPC+5% DMAHDM+30% NACP”).
(5) SBMP primer+7.5% MPC+5% DMAHDM. SBMP adhesive+7.5% MPC+5% DMAHDM+40% NACP (“7.5% MPC+5% DMAHDM+40% NACP”).

### 5.4. Dentine Shear Bond Strength Testing

Extracted caries-free human molars were first sawed to remove the crowns (Isomet, Buehler, Lake Bluff, Ill.), then ground perpendicularly to their longitudinal axes on 320 grit SiC paper until occlusal enamel was completely removed (Cheng et al., 2012b; Zhang et al., 2012). After etching for 15 s and rinsing with water (Antonucci et al., 2009), a primer was applied, and the solvent was removed with an air stream. Then an adhesive was applied and light-cured for 10 s (Optilux-VCL401, Demetron, Danbury, Conn.). Then, a stainless-steel iris with a central opening (diameter=4 mm, thickness=1.5 mm) was held against the adhesive-treated dentin surface. The central opening was filled with a composite (TPH, Caulk/Dentsply, Milford, Del.) and light-cured for 60 s (Antonucci et al., 2009). The bonded specimens were stored in distilled water at 37° C. for 24 h (Cheng et al., 2012b). The dentine shear bond strength, S_{D}, was measured as previously described (Cheng et al., 2012b; Antonucci et al., 2009). A chisel was held parallel to the composite-dentine interface and loaded via a Universal Testing Machine (MTS, Eden Prairie, Minn.) at 0.5 mm/min until the composite-dentine bond failed. S_{D} was calculated as: S_{D}=4P/(πd²), where P is the load at failure, and d is the diameter of the composite (Antonucci et al., 2009). Ten teeth were tested for each group.

### 5.5. Fabrication of Root Dentin Slabs

Root dentin slabs were fabricated using bovine instead of human root dentin, since the development and inhibition rates of caries have been demonstrated to be similar in both calcified tissues (Hara et al., 2003). In addition, bovine teeth are easier to obtain. The specimen preparation procedure followed previous studies (Kaneshiro et al., 2008; Daneshmehr et al., 2008; Diamanti et al., 2011; Giacaman et al., 2012). Extracted bovine incisors with intact roots were used. The root was separated from the crown 1-2 mm below the cemento-enamel junction using a water-cooled diamond saw (Isomet) (Kaneshiro et al., 2008; Daneshmehr et al., 2008). Then, the root dentin slabs were prepared from buccal and lingual root surfaces with a water-cooled diamond saw (Daneshmehr et al., 2008; Diamanti et al., 2011). The slabs were mounted with sticky wax on plexiglas blocks to facilitate handling and were serially polished to remove the cementum and flatten the surfaces (Daneshmehr et al., 2008; Diamanti et al., 2011; Giacaman et al., 2012). The polishing procedure was performed with wetted SiC papers up to 4000 grit. The polished slabs were examined microscopically (20×), to ensure that a smooth dentin surface was obtained (Daneshmehr et al., 2008; Diamanti et al., 2011; Giacaman et al., 2012). The root slabs were cleaned by sonication for 10 min in distilled water (Diamanti et al., 2011).

The final dimensions of the root dentin slabs were approximately 5 mm in length, 3 mm in width, and 2 mm in thickness (Diamanti et al., 2011). One dentin surface of 5 mm×3 mm faced the cementum, while the other 5 mm×3 mm surface faced the pulp. Primer and adhesive were applied according to the recommendations of manufacturer to the dentin surface that faced the cementum with dentinal tubules perpendicular to the surface, in order to simulate the clinical application (Daneshmehr et al., 2008; Diamanti et al., 2011).

### 5.6. Scanning Electron Microscopic (SEM) Examinations

The dentin shear bond strength results showed that the 7.5% MPC+5% DMAHDM+40% NACP group had significantly lower bond strength, while all other groups had bond strength similar to SBMP control. Therefore, the 7.5% MPC+5% DMAHDM+40% NACP group (group 5) was not used in subsequent experiments. Groups 1-4 were used.

Although coating of root dentin surface by adhesives has been shown to be an effective method for prevention of root caries (Gando et al., 2013; Grogono et al., 1994; Kaneshiro et al., 2008; Daneshmehr et al., 2008; Ma et al., 2012). It was reported that the coating thickness of the adhesives was too thin to act as a substantive physical barrier against demineralization (Gando et al., 2013; Kaneshiro et al., 2008), especially after clinical polishing and daily tooth-brushing (Gando et al., 2013). Therefore, the coating thickness of the adhesives is clinically important. In order to measure the thickness of the coated layer of the aforementioned four dentin adhesive coating systems, the root dentin slabs were randomly divided into four groups.

Using one of the aforementioned four adhesive systems, the dentin surface that faced the cementum was covered with a single coat by using the micro-brush supplied in the package, following manufacturer's instructions. First, the root dentin slab surface was etched with etchant for 15 s and rinsed with water, and dried with an air stream (Cheng et al., 2013b; Amaral et al., 2011). Second, a primer was applied to the dentin surface and left for 20 s, and then mild air-blown for 5 s (Cheng et al., 2013b). Third, 20 μL of adhesive was applied and rubbed in for 10 s (Cheng et al., 2013b). The solvent was removed with mild air for 5 s and light-cured for 10 s (Optilux) (Cheng et al., 2013b). Due to the different viscosity of the four adhesive systems, it is expected to produce different coating thickness. The adhesive layer tends to fracture in cutting without composite, hence a composite (TPH) was placed on the cured adhesive and light-cured for 60 s to facilitate the subsequent cutting and the measurement of the adhesive layer thickness (Daneshmehr et al., 2008). The samples were cut through the center perpendicularly to the coated surface via a diamond saw (Isomet) with copious water. The cross-section was polished with increasingly finer SiC papers up to 4000 grit (Kaneshiro et al., 2008; Daneshmehr et al., 2008). After being thoroughly rinsed with water for 10 min, the specimens were air dried and sputter-coated with gold, and examined in SEM (Quanta 200, FEI, Hillsboro, Oreg.). The thickness of the adhesive layer covering the root dentin slab was measured for six specimens, with 4 readings per specimen, for a total of 24 measurements per group.

To examine the interfacial ultrastructure, additional specimens sectioned were treated with 50% phosphoric acid for 30 s and 5% NaOCl for 10 min before being prepared for SEM observation (Kaneshiro et al., 2008). To observe surface texture after coating with adhesive material, root dentin surfaces were coated with one of the adhesives in the same manner as described above (without TPH). The surfaces were then air dried and sputter-coated with gold for SEM observation (Kaneshiro et al., 2008; Daneshmehr et al., 2008).

### 5.7. Measurement of Protein Adsorption

To test the protein-repellent and anti-biofilm properties of groups 1-4, resin disks were fabricated. The cover of a sterile 96-well plate (Costar, Corning Inc., Corning, N.Y.) was used as molds following a previous study (Zhou et al., 2013a). Briefly, 10 μL of a primer was placed in the bottom of each dent of the 96-well plate. After drying with a stream of air, 20 μL of adhesive was applied to the dent and photo-polymerized for 30 s (Optilux), using a Mylar strip covering to obtain a disk of approximately 8 mm in diameter and 0.5 mm in thickness. The cured resin disks were immersed in 200 mL of distilled water and magnetically-stirred with a bar at a speed of 100 rpm for 1 h to remove any uncured monomers, following a previous study (Imazato et al., 1998). The disks were then sterilized with ethylene oxide (Anprolene AN 74i, Andersen, Haw River, N.C.) and de-gassed for 3 d (Zhou et al., 2013a).

The amount of protein adsorbed on resin disks was determined by the micro bicinchoninic acid (BCA) method (Moro et al., 2009; Sibarani et al., 2007). Each disk was immersed in phosphate buffered saline (PBS) for 2 h. The disks then were immersed in bovine serum albumin (BSA) (Sigma-Aldrich) solutions at 37° C. for 2 h. The protein solutions contained BSA at a concentration of 4.5 g/L following previous studies (Moro et al., 2009; Sibarani et al., 2007). The disks then were rinsed with fresh PBS by stirring at a speed of 300 rpm for 5 min (Bellco Glass, Vineland, N.J.), immersed in sodium dodecyl sulfate (SDS) 1 wt % in PBS, and sonicated at room temperature for 20 min to completely detach the BSA adsorbed onto the surface of disk. A protein analysis kit (micro BCA protein assay kit, Fisher Scientific, Pittsburgh, Pa.) was used to determine the BSA concentration in the SDS solution. From the concentration of protein, the amount of protein adsorbed on the resin disk surface was calculated (Moro et al., 2009; Sibarani et al., 2007). Six disks were evaluated for each group.

### 5.8. Saliva Collection for Dental Plaque Microcosm Biofilm Model

Bacteria attachment and biofilm growth behavior on the adhesive systems 1-4 were investigated using a dental plaque microcosm model (Cheng et al., 2012b; Zhang et al., 2012). Saliva is ideal for growing dental plaque microcosm biofilms in vitro, with the advantage of maintaining much of the complexity and heterogeneity of the dental plaque in vivo (McBain, 2009). The saliva for biofilm inoculums was collected from ten healthy adult donors having natural dentition without active caries or periopathology, and without the use of antibiotics within the last 3 months, following previous studies (Cheng et al., 2012b; Zhang et al., 2012). The donors did not brush teeth for 24 h and abstained from food and drink intake for 2 h prior to donating saliva. Stimulated saliva was collected during parafilm chewing and was kept on ice. An equal volume of saliva from each of the ten donors was combined. The saliva was diluted in sterile glycerol to a concentration of 70%, and stored at −80° C. (Cheng et al., 2011).

### 5.9. Dental Plaque Microcosm Biofilm Formation and Live/Dead Assay

The saliva-glycerol stock was added, with 1:50 final dilution, to a growth medium as inoculum (Cheng et al., 2012b; Zhang et al., 2012). The growth medium contained mucin (type II, porcine, gastric) at a concentration of 2.5 g/L; bacteriological peptone, 2.0 g/L; tryptone, 2.0 g/L; yeast extract, 1.0 g/L; NaCl, 0.35 g/L, KCl, 0.2 g/L; CaCl₂, 0.2 g/L; cysteine hydrochloride, 0.1 g/L; hemin, 0.001 g/L; vitamin K1, 0.0002 g/L, at pH 7 (McBain et al., 2005). Each disk was placed into a 24-well plates, 1.5 mL of inoculum was added to each well, and incubated at 37° C. in 5% CO₂ for 8 h. Then, the disks were transferred to new 24-well plates with fresh medium and incubated. After 16 h, the disks were transferred to new 24-well plates with fresh medium and incubated for 24 h. This totaled 2 d of incubation, which was shown to form biofilms on resin disks (Cheng et al., 2011).

Disks with biofilms grown for 2 d were rinsed with phosphate buffered saline (PBS) and live/dead stained using the BacLight live/dead bacterial viability kit (Molecular Probes, Eugene, Oreg.) (Cheng et al., 2012b; Zhang et al., 2012). Live bacteria were stained with Syto 9 to produce a green fluorescence, and bacteria with compromised membranes were stained with propidium iodide to produce a red fluorescence. The stained disks were examined using an inverted epifluorescence microscope (Eclipse TE2000-S, Nikon, Melville, N.Y.). Six specimens were evaluated for each group, three randomly chosen fields of view were photographed from each disk, yielding a total of 18 images for each group.

### 5.10. MTT Assay of Metabolic Activity

Disks with 2 d biofilms were transferred to a new 24-well plate for the MTT [3-(4,5-Dimethylthiazol-2-yl)-2,5-diphenyltetrazolium bromide] assay (Cheng et al., 2012b; Zhang et al., 2012). MTT is a colorimetric assay that measures the enzymatic reduction of MTT, a yellow tetrazole, to formazan. 1 mL of MTT dye (0.5 mg/mL MTT in PBS) was added to each well and incubated at 37° C. in 5% CO₂ for 1 h. During this process, metabolically active bacteria reduced the MTT to purple formazan. Disks were transferred to a new 24-well plate, and 1 mL of dimethyl sulfoxide (DMSO) was added to solubilize the formazan crystals. The plate was incubated for 20 min with gentle mixing at room temperature in the dark. Then, 200 μL of the DMSO solution from each well was collected, and its absorbance at 540 nm was measured via a microplate reader (SpectraMax M5, Molecular Devices, Sunnyvale, Calif.). A higher absorbance is related to a higher formazan concentration, which indicates a higher metabolic activity in the biofilm on the disk (Cheng et al., 2012b; Zhang et al., 2012).

### 5.11. Lactic Acid Production by Biofilms Adherent on Resin Disks

Disks with 2 d biofilms were rinsed with cysteine peptone water (CPW) to remove loose bacteria (Cheng et al., 2012b). The disks were transferred to 24-well plates containing buffered peptone water (BPW) plus 0.2% sucrose. The disks were incubated in 5% CO₂ at 37° C. for 3 h to allow the biofilms to produce acid (Cheng et al., 2012b). The BPW solutions were collected for lactate analysis using an enzymatic method. The 340-nm absorbance of BPW was measured with the microplate reader. Standard curves were prepared using a standard lactic acid (Supelco, Bellefonte, Pa.).

### 5.12. Colony-Forming Unit (CFU) Counts

Disks with 2 d biofilms were transferred into tubes with 2 mL CPW, and the biofilms were harvested by sonication and vortexing (Fisher, Pittsburgh, Pa.) (Cheng et al., 2012b; Zhang et al., 2012). Three types of agar plates were used to measure the CFU counts. First, tryptic soy blood agar culture plates were used to determine total microorganisms (McBain et al., 2005). Second, mitis salivarius agar (MSA) culture plates containing 15% sucrose were used to determine total streptococci (Lima et al., 2009). This is because MSA contains selective agents including crystal violet, potassium tellurite and trypan blue, which inhibit most Gram-negative bacilli and most Gram-positive bacteria except streptococci, thus enabling the streptococci to grow (Lima et al., 2009). Third, cariogenic mutans streptococci is known to be resistant to bacitracin, and this property is used to isolate mutans streptococci from the highly heterogeneous oral microflora (McBain et al., 2005). Therefore, MSA agar culture plates plus 0.2 units of bacitracin per mL was used to determine mutans streptococci (McBain et al., 2005). The bacterial suspensions were serially diluted and spread onto agar plates for CFU analysis.

### 5.13. Statistical Analysis

One-way and two-way analyses of variance (ANOVA) were performed to detect the significant effects of the variables. Tukey's multiple comparison test was used to compare the data at a p value of 0.05.

### Results

Root caries prevalence, incidence and severity tend to increase with age and it seems likely that the disease will become a public health issue in the near future due to the rapid increase in the elderly population and a greater number of teeth and exposed root surfaces in the mouths of these individuals (Griffin et al., 2004). Adhesive systems, which can provide a physical barrier through the formation of a hybrid layer, have been proved as an effective method to protect exposed roots from acid attack (Gando et al., 2013; (Grogono et al., 1994; (Kaneshiro et al., 2008; Daneshmehr et al., 2008; Ma et al., 2012). However, there has been no report on dentin adhesives that possess protein-repellent, antibacterial and remineralization capabilities for preventing root caries. Based on this knowledge, a multifunctional adhesive coating system for preventing root caries by combining MPC for protein-repellent ability, with DMAHDM for antibacterial potency, and NACP for remineralization capability into dental adhesive resin was developed as reported herein.

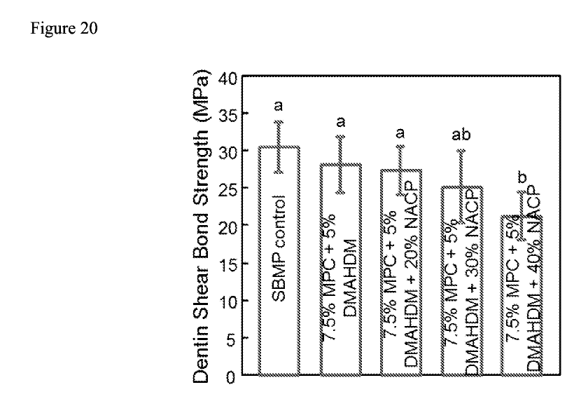

FIG. 20 plots the dentin shear bond strengths (mean±sd; n=10). Groups 1-4 had dentin bond strengths that were not significantly different from each other (p>0.1). Group 5 had a lower strength than SBMP control (p<0.05). Therefore, incorporating 7.5% MPC, 5% DMAHDM, and NACP up to 30% into SBMP did not significantly alter the dentin shear bond strength.

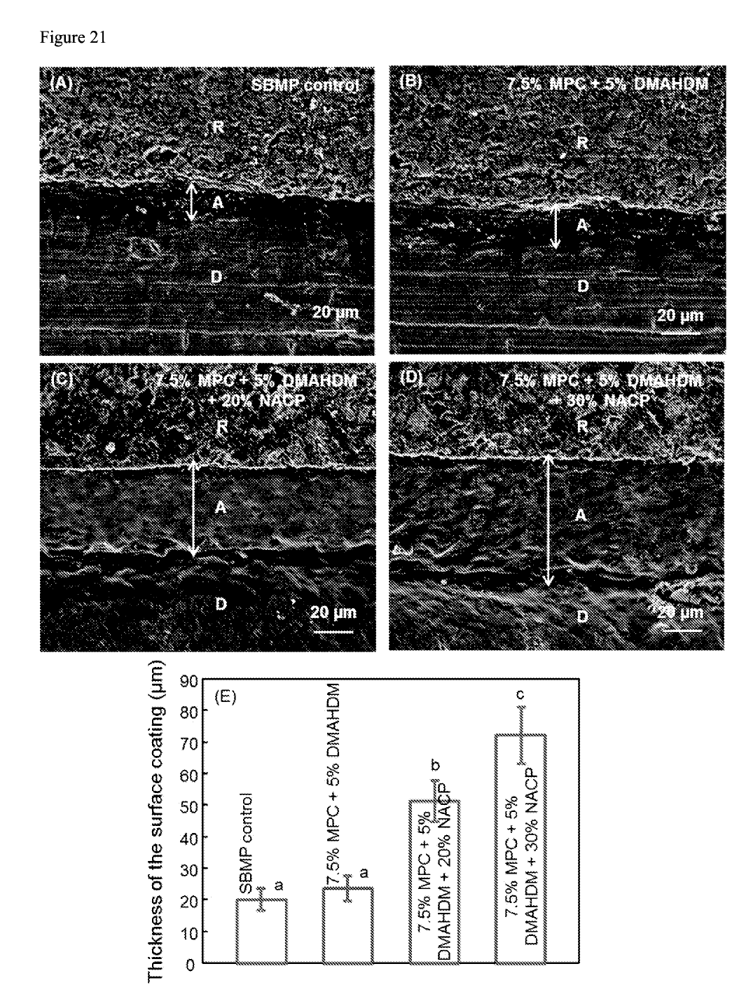

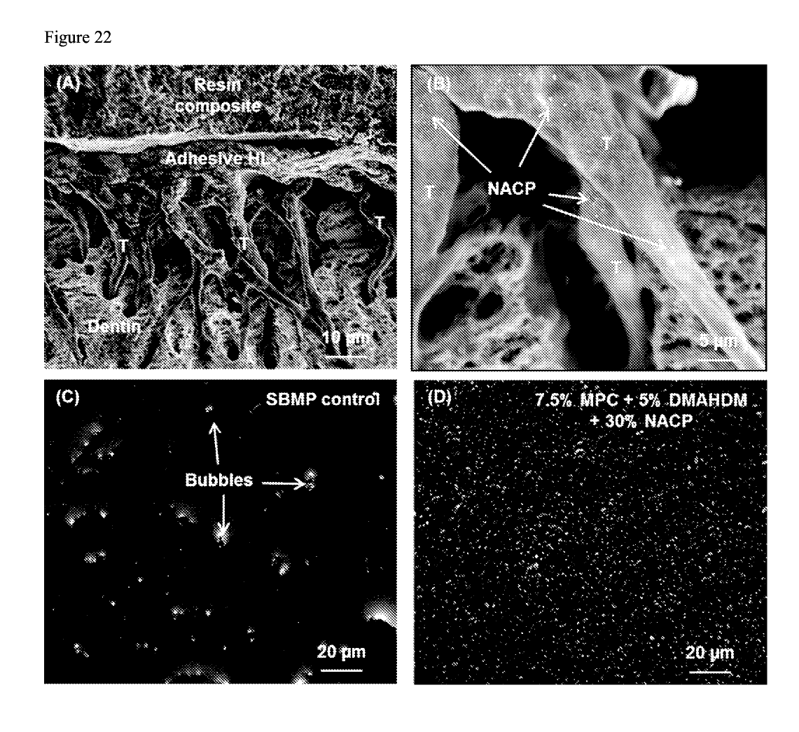

Typical SEM images of the coating thickness of each adhesive were shown in FIG. 21 (A-D). The mean values of adhesive coating thickness for each adhesive are plotted in FIG. 21 (E) (mean±sd; n=6). Adding NACP into the adhesive greatly increased the coating thickness. The coating thickness of the 7.5% MPC+5% DMAHDM+30% NACP group was greater than the other groups (p<0.05).

Representative SEM images of the dentin-adhesive interfaces and surface texture are shown in FIG. 22. The example shown in (A) was for the 7.5% MPC+5% DMAHDM+30% NACP group. Numerous resin tags “T” from well-filled dentinal tubules were visible in (A). “HL” refers to the hybrid layer between the adhesive and the underlying mineralized dentin. At a higher magnification, the NACP were visible in (B) with 30% NACP. Arrows in (B) indicate examples of NACP infiltrated into the dentinal tubules. In (C), some bubble-shaped deficiencies were observed on the cured SBMP control adhesive. In contrast, (D) 7.5% MPC+5% DMAHDM+30% NACP group showed uniform surfaces and the exposed dentin was hermetically sealed with resinous material.

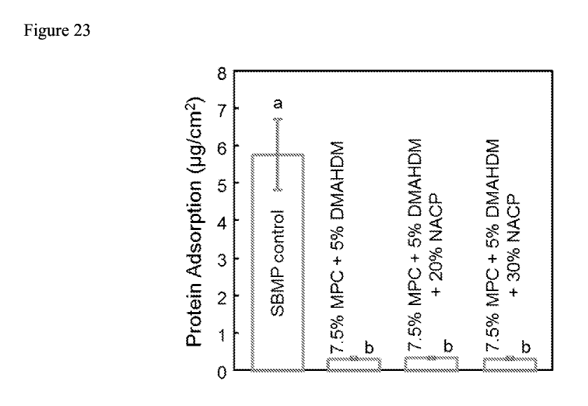

FIG. 23 plots the amount of protein adsorption of each group (mean±sd; n=6). Incorporating MPC into adhesive resin significantly decreased the amount of protein adsorption. The SBMP control had the highest amount of protein adsorption, that was nearly 18-fold higher than the other groups (p<0.05).

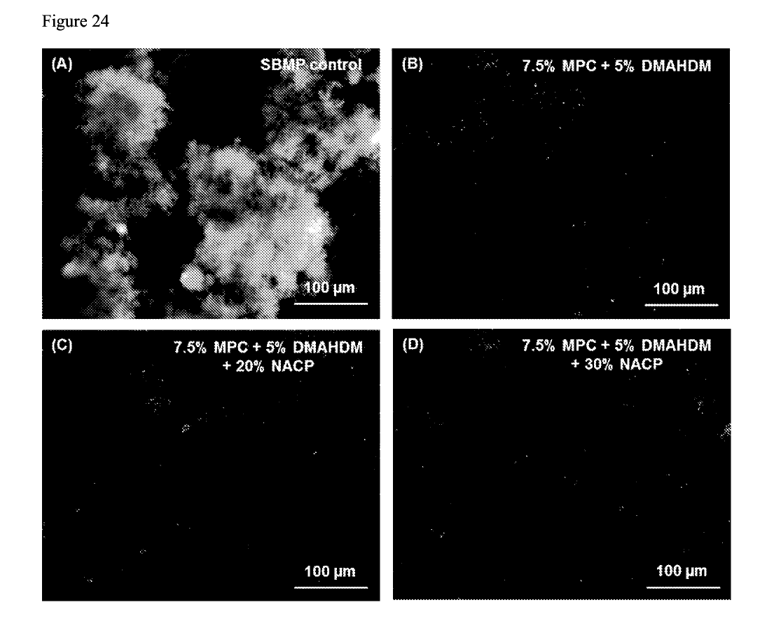

Typical live/dead staining images of 2 d biofilms grown on resin disks are shown in FIG. 24. The SBMP control was fully covered by primarily live bacteria. In contrast, (B), (C) and (D) showed noticeably decreased in bacterial adhesion, and the biofilms consisted of primarily dead bacteria.

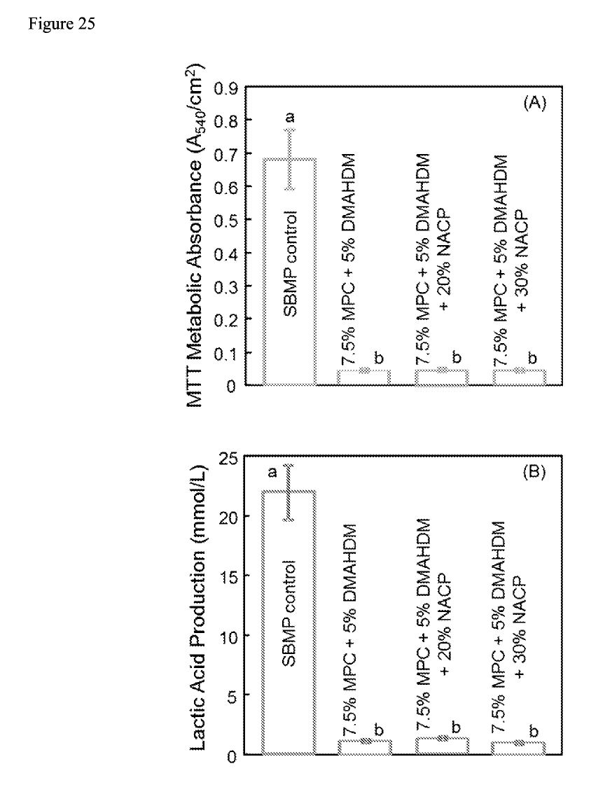

FIG. 25 plots: (A) metabolic activity, and (B) lactic acid production (mean±sd; n=6). Biofilms on SBMP control had the highest metabolic activity and the most lactic acid production. MPC-DMAHDM-NACP containing resin greatly decreased the metabolic activity and lactic acid production of biofilms, compared to the SBMP control (p<0.05).

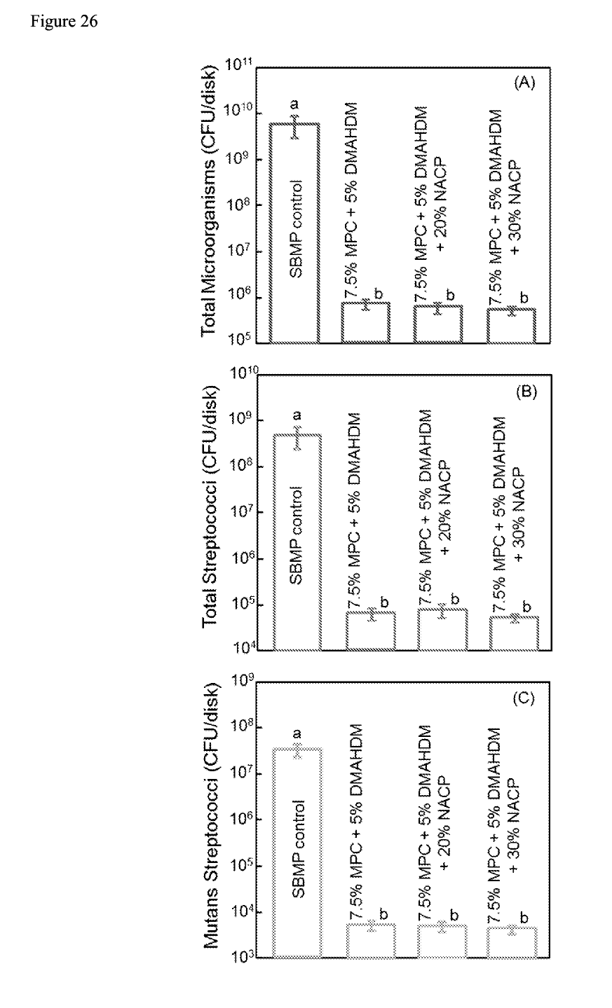

FIG. 26 plots the 2 d biofilm CFU for: (A) total microorganisms, (B) total streptococci, and (C) mutans streptococci (mean±sd; n=6). The values are shown in a log scale. All three CFU counts on MPC-DMAHDM-NACP containing resin were more than 4 orders of magnitude lower than that of SBMP control (p<0.05).

In the present study, the four dentin adhesive coating systems were applied in the same manner according to the instructions of the manufacturer, thereby diminishing the influence of application. A single coat of SBMP control created a thin layer of about 20 μm (FIG. 21), which might be too thin to produce a protective layer (Gando et al., 2013; Kaneshiro et al., 2008; Kuramoto et al., 2005). Triple coat of SBMP control may create enough coating thickness as reported by previous studies (Kaneshiro et al., 2008; Daneshmehr et al., 2008). However, the coating layer should be light-cured after each coating (Daneshmehr et al., 2008; Kuramoto et al., 2005), thereby consuming too much of dentist's time. In addition, the more amount of adhesive was applied, the more thicker of coating layer was produced. Nonetheless, in the oral cavity, due to the gravity, if too much of adhesive was applied onto the exposed root surface, the excess adhesive would flow away to the undesired place, such as gingiva and mucosa, thus leading to contamination. In contrast, incorporating NACP into adhesive increased the viscosity of adhesive paste, thereby increasing the coating thickness. A single coat of 7.5% MPC+5% DMAHDM+30% NACP resin had the thickest coating layer of nearly 70 μm (FIG. 21), which was greater than SBMP control, and was comparable to the results of previous studies (Kaneshiro et al., 2008; Daneshmehr et al., 2008). Covering the exposed dentin surfaces using the coating materials and sealing dentinal tubules can also potentially reduce dentin hypersensitivity and inhibit root caries (Kaneshiro et al., 2008; Daneshmehr et al., 2008). The surface texture of the adhesive-coated root dentin slab was observed by SEM. The coating layers of SBMP control showed a porous appearance, while tighter ones were produced by 7.5% MPC+5% DMAHDM+30% NACP group, as shown in FIGS. 22(C and D). However, the thickness and surface texture of the coating layer should not be the parameter to determine its acid resistance (Kaneshiro et al., 2008). Complete penetration of resins into the etched tooth substrate contributes to producing more impermeable layers (Kaneshiro et al., 2008). The ultrastructure of the dentin-adhesive interfaces was observed by SEM. Numerous resin tags were visible in 7.5% MPC+5% DMAHDM+30% NACP group as shown in FIG. 22(A). Resin tags were formed by adhesive filling into dentinal tubules. The hybrid layer between the adhesive and the underlying mineralized dentine was formed via bonding agent infiltrating into the demineralized collagen layer (Zhang et al., 2013; (Melo et al., 2013). Accordingly, the MPC-DMAHDM-NACP containing adhesive not only can produce a moderate coating thickness on the dentin surface, but also can form the integrate hybridized layer with dentin.

Besides the physical barrier function, there are four advantages of this novel multifunctional adhesive coating system. First, incorporating MPC into adhesive resin significantly decreased the amount of protein adsorption (FIG. 23), thereby reducing the bacterial adhesion and inhibiting the biofilm formation. Second, QAMs had bacteriolysis effects, because their positively charged quaternary amine N⁺ can attract the negatively charged cell membrane of bacteria, which could disrupt the cell membrane and cause cytoplasmic leakage (Namba et al., 2009). DMAHDM was an antibacterial quaternary ammonium monomer with an alkyl chain length of 16 which possessed a strong antibacterial potency (Zhou et al., 2013a). Third, one potential limitation of adhesives containing QAS monomers is that the deposit of salivary proteins on surfaces could decrease the efficacy of “contact-inhibition”, thus reducing the antibacterial potency (Namba et al., 2009). Previous study demonstrated that a saliva-derived protein film on the cationic antibacterial surfaces reduced the original bactericidal effect (Müller et al., 2009). Because the MPC-containing resin can greatly reduce the protein adsorption, there would be much less protein on the resin and hence more direct resin-bacteria contact, thereby might enhance the antibacterial effect of DMAHDM. Indeed, the results in FIGS. 24 to 26 confirmed that the combination of MPC with DMAHDM into primer and adhesive greatly reduced the microcosm biofilm viability, metabolic activity, CFU and lactic acid production, compared to the commercial control. The last, the MPC-DMAHDM-NACP containing adhesive is not only antibacterial, but also has Ca and P ion release and remineralization capabilities. Several recent studies evaluated the ion release and remineralization properties of NACP nanocomposites (Dickens et al., 2003; Langhorst et al., 2009; Xu et al., 2006; Xu et al., 2010: Xu et al., 2011; Moreau et al., 2011). The NACP nanocomposite was “smart” and increased the Ca and P ion release at a cariogenic pH of 4, when these ions would be most needed to inhibit caries (Xu et al., 2006; Xu et al., 2010). A previous study showed that a composite with 20% NACP released Ca and P ions to levels comparable to traditional CaP composites shown to effectively remineralize tooth lesions (Xu et al., 2011). Another study showed that a composite with 30% NACP neutralized an acid attack, and raised the solution pH from a cariogenic pH of 4 to a safe pH of 6.5 (Moreau et al., 2011). Therefore, the incorporation of 20% or 30% NACP into the adhesive is expected to provide acid neutralization and remineralization benefits.

Another important issue is the long-term durability of the MPC-DMAHDM-NACP containing adhesive. In the present study, the dentin shear bond strengths were measured using coronal dentin instead of root dentin. The results showed that the MPC-DMAHDM-NACP containing adhesive formed physical barrier without compromising the dentin bond strength, thus can resist the mechanical stress in vivo. Regarding the durability of the protein-repellent capability, it was reported that the MPC-modified surface layer formed by photo-induced graft polymerization was resistant to mechanical stresses (Kyomoto et al., 2009a). MPC was grafted onto the surface through covalent bonding, and the strong C—C bonding may offer durable resistance to protein adsorption (Kyomoto et al., 2009a). Furthermore, previous study showed that the MPC-grafted layer provided high lubricity for the surface (Kyomoto et al., 2009b). This lubrication may result in durability against the mechanical stress caused by brushing, thus offering sufficient durability for clinical application. In the present study, the SBMP primer and adhesive contained HEMA and copolymer of acrylic and itaconic acids, which could copolymerize with MPC when the adhesive was light-cured. The MPC was mixed into and copolymerized with the entire volume of the adhesive resin, so that MPC will be present even after polishing and daily tooth-brushing to continue to repel proteins. Therefore, the MPC protein-repellent activity is expected to be durable.

Regarding the durability of the antibacterial function, previous studies showed that the incorporation of QAMs in resins yielded a long-term antibacterial activity, because the antibacterial agent was copolymerized with the resin by forming a covalent bonding with the polymer network (Imazato et al., 2003a; Imazato et al., 2003c; Imazato et al., 2007; Imazato et al., 2009). Therefore, the antibacterial agent was immobilized in the resin matrix, and was not released or lost over time, thus providing a durable antibacterial capability (Imazato et al., 2003a; Imazato et al., 2003c; Imazato et al., 2007; Imazato et al., 2009). A recent study on bonding agents containing dimethylaminododecyl methacrylate demonstrated that the anti-biofilm properties had no significant decrease in water-aging from 1 day to 6 months (Zhang et al., 2013). In the present study, the main reason of using NACP, and not traditional CaP fillers, was that NACP could release more Ca and P ions at lower filler levels (Xu et al., 2011; Moreau et al., 2011). This is important because a low filler level could be used for adhesives so that the adhesive could maintain a moderate viscosity and the ability to flow into dentinal tubules. In addition, NACP with a size of about 100 nm could infiltrate into dentinal tubules easily as shown in FIG. 22(B), which may provide long-term remineralization property.

All documents, books, manuals, papers, patents, published patent applications, guides, abstracts and other reference materials cited herein are incorporated by reference in their entirety. While the foregoing specification teaches the principles of the present invention, with examples provided for the purpose of illustration, it will be appreciated by one skilled in the art from reading this disclosure that various changes in form and detail can be made without departing from the true scope of the invention.

### CITED DOCUMENTS

Amaral N G, Rezende M L, Hirata F, Rodrigues M G, Sant'ana A C, Greghi S L. (2011). Comparison among four commonly used demineralizing agents for root conditioning: a scanning electron microscopy. Journal of Applied Oral Science 19:469-75.
Amerongen A V, Bolscher J G, Veerman E C. (1995). Salivary mucins: protective functions in relation to their diversity. Glycobiology 5(8): 733-740.
Antonucci J M, O'Donnell J N, Schumacher G E, Skrtic D (2009). Amorphous calcium phosphate composites and their effect on composite-adhesive-dentin bonding. J Adhes Sci Technol 23:1133-1147.
Antonucci J M, Zeiger D N, Tang K, Lin-Gibson S, Fowler B O, Lin N J (2012). Synthesis and characterization of dimethacrylates containing quaternary ammonium functionalities for dental applications. Dent Mater 28:219-228.
Bayne S C, Thompson J Y, Swift Jr. E J, Stamatiades P, Wilkerson M (1998). A characterization of first-generation flowable composites. J Am Dent Assoc 129:567-577.
Beyth N, Yudovin-Farber I, Bahir R, Domb A J, Weiss E I. (2006). Antibacterial activity of dental composites containing quaternary ammonium polyethylenimine nanoparticles against Streptococcus mutans. Biomaterials 27:3995-4002.
Burne R A (1998). Oral Streptococci . . . Products of their environment. J Dent Res 77:445-452.
Carlén A, Nikdel K, Wennerberg A, Holmberg K, Olsson J (2001). Surface characteristics and in vitro biofilm formation on glass ionomer and composite resin. Biomaterials 22:481-487.
Cenci M S, Pereira-Cenci T, Cury J A, ten Cate J M (2009). Relationship between gap size and dentine secondary caries formation assessed in a microcosm biofilm model. Caries Res 43:97-102.
Chai Z, Li F, Fang M, Wang Y, Ma S, Xiao Y, Huang L, Chen J (2011). The bonding property and cytotoxicity of a dental adhesive incorporating a new antibacterial monomer. Oral Rehab 38:849-856.
Cheng L, Exterkate R A, Zhou X, Li J, ten Cate J M (2011). Effect of galla chinensis on growth and metabolism of microcosm biofilms. Caries Res 45:87-92.
Cheng L, Weir M D, Xu H H K, Antonucci J M, Kraigsley A M, Lin N J, Lin-Gibson S, Zhou X D (2012a). Antibacterial amorphous calcium phosphate nanocomposite with quaternary ammonium salt and silver nanoparticles. Dent Mater 28:561-572
Cheng L, Zhang K, Melo M A S, Weir M D, Zhou X D, Xu H H K (2012b). Anti-biofilm dentin primer with quaternary ammonium and silver nanoparticles. J Dent Res 91:598-604.
Cheng L, Weir M D, Zhang K, Xu S M, Q Chen, Zhou X D, Xu H H K (2012c). Antibacterial nanocomposite with calcium phosphate and quaternary ammonium. J Dent Res 91:460-466.
Cheng L, Weir M D, Zhang K et al. (2012d). Dental plaque microcosm biofilm behavior on calcium phosphate nanocomposite with quaternary ammonium. Dent Mater 28(8): 853-862.
Cheng L, Weir M D, Zhang K, Arola D D, Zhou X, Xu H H. (2013a). Dental primer and adhesive containing a new antibacterial quaternary ammonium monomer dimethylaminododecyl methacrylate. Journal of Dentistry 41:345-55.
Cheng L, Zhang K, Weir M D, Liu H, Zhou X, Xu H H. (2013b). Effects of antibacterial primers with quaternary ammonium and nano-silver on Streptococcus mutans impregnated in human dentin blocks. Dental Materials 29:462-72.
Chow L C, Sun L, Hockey B (2004). Properties of nanostructured hydroxyapatite prepared by a spray drying technique. J Res NIST 109:543-551.
Daneshmehr L, Matin K, Nikaido T, Tagami J. (2008). Effects of root dentin surface coating with all-in-one adhesive materials on biofilm adherence. Journal of Dentistry 36:33-41.
Deng D M, ten Cate J M (2004). Demineralization of dentin by Streptococcus mutans biofilms grown in the constant depth film fermentor. Caries Res 38:54-61.
Deng D M, van Loveren C, ten Cate J M (2005). Caries-preventive agents induce remineralization of dentin in a biofilm model. Caries Res 39:216-223.
Deligeorgi V, Mjor I A, Wilson N H (2001). An overview of reasons for the placement and replacement of restorations. Prim Dent Care 8:5-11.
Diamanti I, Koletsi-Kounari H, Mamai-Homata E, Vougiouklakis G. (2011). In vitro evaluation of fluoride and calcium sodium phosphosilicate toothpastes, on root dentine caries lesions. Journal of Dentistry 39:619-28.
Dickens S H, Flaim G M, Takagi S. (2003). Mechanical properties and biochemical activity of remineralizing resin-based Ca—PO4 cements. Dental Materials 9:558-66.
Donlan R M, Costerton J W (2002). Biofilms: survival mechanisms of clinically relevant microorganisms. Clinical Microbiology Reviews 15:167-93.
Drummond J L, Bapna M S (2003). Static and cyclic loading of fiber-reinforced dental resin. Dent Mater 19:226-231.
Drummond J L (2008). Degradation, fatigue, and failure of resin dental composite materials. J Dent Res 87:710-719.
Featherstone J D B (2000). The science and practice of caries prevention. J Am Dent Assoc 131:887-899.
Featherstone J D B (2004). The continuum of dental caries—Evidence for a dynamic disease process. J Dent Res 83: C39-C42.
Ferracane J L (1995). Current trends in dental composites. Crit Rev Oral Biol Med 6:302-318.
Frost P M (2002). An audit on the placement and replacement of restorations in a general dental practice. Prim Dent Care 9:31-36.
Gando I, Ariyoshi M, Ikeda M, Sadr A, Nikaido T, Tagami J. (2013). Resistance of dentin coating materials against abrasion by toothbrush. Dental Materials Journal 32:68-74.
Giacaman R A, Muñoz M J, Ccahuana-Vasquez R A, Muñoz-Sandoval C, Cury J A. (2012). Effect of fluoridated milk on enamel and root dentin demineralization evaluated by a biofilm caries model. Caries Research 46:460-6.
Griffin S O, Griffin P M, Swann J L, Zlobin N. (2004). Estimating rates of new root caries in older adults. Journal of Dental Research 83:634-8.
Grogono A L, Mayo J A. (1994). Prevention of root caries with dentin adhesives. American Journal of Dentistry 7:89-90.
Hara A T, Queiroz C S, Paes Leme A F, Serra M C, Cury J A. (2003). Caries progression and inhibition in human and bovine root dentine in situ. Caries Research 37:339-44.
He J, Soderling E, Osterblad M, Vallittu P K, Lassila L V J. (2011). Synthesis of methacrylate monomers with antibacterial effects against S. mutans. Molecules 16:9755-63.
Hildebrandt G H, Bretz W A (2006). Comparison of culture media and chairside assays for enumerating mutans streptococci. J Appl Microbiol 100:1339-1347.
Huang L, Xiao Y H, Xing X D, Li F, Ma S, Qi L L, Chen J (2011). Antibacterial activity and cytotoxicity of two novel cross-linking antibacterial monomers on oral pathogens. Arch Oral Biol 56:367-373.
Imazato S, Torii M, Tsuchitani Y, McCabe J F, Russell R R B (1994). Incorporation of bacterial inhibitor into resin composite. J Dent Res 73; 1437-1443.
Imazato S, Ehara A, Torii M, Ebisu S (1998). Antibacterial activity of dentine primer containing MDPB after curing. J Dent 26:267-271.
Imazato S, Kinomoto Y, Tarumi H, Ebisu S, Tay F R. (2003a). Antibacterial activity and bonding characteristics of an adhesive resin containing antibacterial monomer MDPB. Dental Materials 19:313-9.
Imazato S, Ebi N, Takahashi Y, Kaneko T, Ebisu S, Russell R R. (2003b). Antibacterial activity of bactericide-immobilized filler for resin-based restoratives. Biomaterials 24:3605-9.
Imazato S (2003c). Review: Antibacterial properties of resin composites and dentin bonding systems. Dent Mater 19:449-457.
Imazato S, Kuramoto A, Takahashi Y, Ebisu S, Peters M C (2006). In vitro antibacterial effects of the dentin primer of Clearfil Protect Bond. Dent Mater 22:527-532.
Imazato S, Tay F R, Kaneshiro A V, Takahashi Y, Ebisu S (2007). An in vivo evaluation of bonding ability of comprehensive antibacterial adhesive system incorporating MDPB. Dent Mater 23:170-176.
Imazato S. (2009). Bio-active restorative materials with antibacterial effects: new dimension of innovation in restorative dentistry. Dental Materials Journal 28:11-9.
Ishihara K, Ueda T, Nakabayashi N. (1990). Preparation of phospholipid polymers and their properties as polymer hydrogel membranes. Polymer Journal 22:355-60.
Ishihara K, Ziats N P, Tierney B P et al. (1991). Protein adsorption from human plasma is reduced on phospholipid polymers. J Biomed Mater Res 25(11): 1397-1407.
Kaneshiro A V, Imazato S, Ebisu S, Tanaka S, Tanaka Y, Sano H. (2008). Effects of a self-etching resin coating system to prevent demineralization of root surfaces. Dental Materials 24:1420-7.
Kolenbrander P E, London J (1993). Adhere today, here tomorrow: oral bacterial adherence. J. Bacteriol. 175:3247-52.
Kramer N, Garcia-Godoy F, Reinelt C, Frankenberger R (2006). Clinical performance of posterior compomer restorations over 4 years. Am J Dent 19:61-66.
Kuramoto A, Imazato S, Walls A W, Ebisu S. (2005). Inhibition of root caries progression by an antibacterial adhesive. Journal of Dental Research 84:89-93.
Kyomoto M, Moro T, Miyaji F, Hashimoto M, Kawaguchi H, Takatori Y, Nakamura K, Ishihara K. (2009a). Effects of mobility/immobility of surface modification by 2-methacryloyloxyethyl phosphorylcholine polymer on the durability of polyethylene for artificial joints. Journal of Biomedical Materials Research. Part A 90:362-71.
Kyomoto M, Ishihara K. (2009b). Self-initiated surface graft polymerization of 2-methacryloyloxyethyl phosphorylcholine on poly(ether ether ketone) by photoirradiation. ACS Applied Materials & Interfaces 1:537-42.
Langhorst S E, O'Donnell S N R, Skrtic D. (2009). In vitro remineralization of enamel by polymeric amorphous calcium phosphate composite: quantitative microradiographic study. Dental Materials 25:884-91.
Lendenmann U, Grogan J, Oppenheim F G (2000). Saliva and dental pellicle—a review. Advances in Dental Research 14:22-8.
Li F, Chen J, Chai Z, Zhang L, Xiao Y, Fang M, Ma S (2009). Effects of a dental adhesive incorporating antibacterial monomer on the growth, adherence and membrane integrity of Streptococcus mutans. J Dent 37:289-296.
Li F, Weir M D, Fouad A F et al. (2014). Effect of salivary pellicle on antibacterial activity of novel antibacterial dental adhesives using a dental plaque microcosm biofilm model. Dent Mater 30(2): 182-191.
Lim B S, Ferracane J L, Sakaguchi R L, Condon J R (2002). Reduction of polymerization contraction stress for dental composites by two-step light-activation. Dent Mater 18:436-444.
Lima J P, Sampaio de Melo M A, Borges F M, Teixeira A H, Steiner-Oliveira C, Nobre Dos Santos M, et al (2009). Evaluation of the antimicrobial effect of photodynamic antimicrobial therapy in an in situ model of dentine caries. Eur J Oral Sci. 117:568-74.
Loesche W J (1986). Role of Streptococcus mutans in human dental decay. Microbiological Reviews 50:353-380.
Lu H, Stansbury J W, and C. N. Bowman C N (2005). Impact of curing protocol on conversion and shrinkage stress. J Dent Res 84:822-826.
Ma S, Imazato S, Chen J H, Mayanagi G, Takahashi N, Ishimoto T, Nakano T. (2012). Effects of a coating resin containing S-PRG filler to prevent demineralization of root surfaces. Dental Materials Journal 31:909-15.
McBain A J (2009). In vitro biofilm models: an overview. Adv Appl Microbiol 69:99-132.
McBain A J, Sissons C, Ledder R G, Sreenivasan P K, De Vizio W, Gilbert P (2005). Development and characterization of a simple perfused oral microcosm. J Appl Microbiol 98:624-634.
Melo M A, Cheng L, Zhang K, Weir M D, Rodrigues L K, Xu H H. (2013). Novel dental adhesives containing nanoparticles of silver and amorphous calcium phosphate. Dental Materials 29:199-210.
Menschutkin, N. Z (1890). Physik. Chemie 5, 589.
Mjör I A, Moorhead J E, Dahl J E (2000). Reasons for replacement of restorations in permanent teeth in general dental practice. International Dent J 50:361-366.
Moreau J L, Sun L, Chow L C, Xu H H K (2011). Mechanical and acid neutralizing properties and inhibition of bacterial growth of amorphous calcium phosphate dental nanocomposite. J Biomed Mater Res Part B 2011; 98: 80-88.
Moro T, Kawaguchi H, Ishihara K, Kyomoto M, Karita T, Ito H. (2009). Wear resistance of artificial hip joints with poly(2-methacryloyloxyethyl phosphorylcholine) grafted polyethylene: comparisons with the effect of polyethylene cross-linking and ceramic femoral heads. Biomaterials 30:2995-3001.
Müller R, Eidt A, Hiller K A, Katzur V, Subat M, Schweikl H, Imazato S, Ruhl S, Schmalz G (2009). Influences of protein films on antibacterial or bacteria-repellent surface coatings in a model system using silicon wafers. Biomaterials 30:4921-9.
Murata H, Koepsel R R, Matyjaszewski K, Russell A J. (2007). Permanent, non-leaching antibacterial surfaces-2: how high density cationic surfaces kill bacterial cells. Biomaterials 28:4870-9.
Namba N, Yoshida Y, Nagaoka N, Takashima S, Matsuura-Yoshimoto K, Maeda H, Van Meerbeek B, Suzuki K, Takashiba S. (2009). Antibacterial effect of bactericide immobilized in resin matrix. Dental Materials 25:424-30.
Park J, Ye Q, Topp E, Misra A, Kieweg S L, Spencer P (2009). Water sorption and dynamic mechanical properties of dentin adhesives with a urethane-based multifunctional methacrylate monomer. Dent Mater 25:1569-1575.
Park J H, Tanabe Y, Tinanoff N, Turng B F, Lilli H, Minah G E (2006). Evaluation of microbiological screening systems using dental plaque specimens from young children aged 6-36 months. Caries Res. 40:277-80.
Ruddell D E, M. M. Maloney M M, Thompson J Y (2002). Effect of novel filler particles on the mechanical and wear properties of dental composites. Dent Mater 18:72-80.
Sakaguchi R L (2005). Review of the current status and challenges for dental posterior restorative composites: clinical, chemistry, and physical behavior considerations. Dent Mater 21:3-6.
Sarrett D C (2005). Clinical challenges and the relevance of materials testing for posterior composite restorations. Dent Mater 21:9-20.
Sibarani J, Takai M, Ishihara K. (2007). Surface modification on microfluidic devices with 2-methacryloyloxyethyl phosphorylcholine polymers for reducing unfavorable protein adsorption. Colloids and Surfaces B: Biointerfaces 54:88-93
Stoodley P, Wefel J, Gieseke A, deBeer D, von Ohle C (2008). Biofilm plaque and hydrodynamic effects on mass transfer, fluoride delivery and caries. J Am Dent Assoc 139:1182-1190.
Svanberg M, Mjör I A, Orstavik D (1990). Mutans streptococci in plaque from margins of amalgam, composite, and glass-ionomer restorations. J Dent Res 69:861-864.
Takahashi Y, Imazato S, Russell R R B, Noiri Y, Ebisu S (2004). Influence of resin monomers on growth of oral streptococci. J Dent Res 83:302-306.
Takahashi N, Iwasa F, Inoue Y, Morisaki H, Ishihara K, Baba K. (2014). Evaluation of the durability and antiadhesive action of 2-methacryloyloxyethyl phosphorylcholine grafting on an acrylic resin denture base material. J Prosthet Dent. 112(2):194-203.
Totiam P, Gonzalez-Cabezas C, Fontana M R, Zero D T (2007). A new in vitro model to study the relationship of gap size and secondary caries. Caries Res 41:467-473.
Van Houte J (1994). Role of micro-organisms in caries etiology. J Dent Res 73:672-681.
Watts D C, Marouf A S, Al-Hindi A M (2003). Photo-polymerization shrinkage-stress kinetics in resin-composites: methods development. Dent Mater 19:1-11.
Xu H H K, Sun L, Weir M D, Antonucci J M, Takagi S, Chow L C (2006). Nano dicalcium phosphate anhydrous-whisker composites with high strength and Ca and PO₄ release. J Dent Res 85:722-727.
Xu H H, Weir M D, Sun L, Moreau J L, Takagi S, Chow L C, Antonucci J M. (2010). Strong nanocomposites with Ca, PO4 and F release for caries inhibition. Journal of Denial Research 8919-28.
Xu H H K, Moreau J L, Sun L, Chow L C (2011). Nanocomposite containing amorphous calcium phosphate nanoparticles for caries inhibition. Dent Mater 27:762-769.
Zero D T (1995). In situ caries models. Adv Dent Res 9:214-230.
Zhang K, Melo M A S, Cheng L, Weir M D, Bai Y X, Xu H H K (2012). Effect of quaternary ammonium and silver nanoparticle-containing adhesives on dentin bond strength and dental plaque microcosm biofilms. Dent Mater 28:842-852.
Zhang K, Cheng L, Wu E J, Weir M D, Bai Y, Xu H H. (2013). Effect of water-aging on dentin bond strength and anti-biofilm activity of bonding agent containing antibacterial monomer dimethylaminododecyl methacrylate. Journal of Dentistry 41:504-13.
Zhou H, Li F, Weir M D, Xu H H. (2013a). Dental plaque microcosm response to bonding agents containing quaternary ammonium methacrylates with different chain lengths and charge densities. Journal of Dentistry 41:1122-31.
Zhou C, Weir M D, Zhang K, Deng D, Cheng L, Xu H H. (2013b). Synthesis of new antibacterial quaternary ammonium monomer for incorporation into CaP nanocomposite. Dental Materials 29:859-70.

1-114. (canceled)

115. A protein-repellant dental material comprising a protein-repellant agent, wherein the protein-repellant agent is one or more of 2-methacryloyloxyethyl phosphorylcholine (MPC), poly(hydroxyethyl methacrylate) (HEMA) or a derivative thereof, or poly(N-isopropylacrylamide) or a derivative thereof.

116. The protein-repellant dental material of claim 115, wherein the material is selected from the group consisting of a protein-repellant dental primer, a protein-repellant dental adhesive, a protein-repellant dental resin, a protein-repellant dental composite, a protein-repellant dental cement, a protein-repellant dental sealant, a protein-repellant dental base, and a protein-repellant dental liner.

117. The protein-repellant dental material of claim 116, wherein the protein-repellant dental material is a protein-repellant dental primer comprising a dental primer and the protein-repellant agent.

118. The protein-repellant dental material of claim 116, wherein the protein-repellant dental material is a protein-repellant dental adhesive comprising a dental adhesive and the protein-repellant agent.

119. The protein-repellant dental material of claim 116, wherein the protein-repellant dental material is a protein-repellant dental resin comprising a dental resin and the protein-repellant agent.

120. The protein-repellant dental material of claim 116, wherein the protein-repellant dental material is a protein-repellant dental composite comprising a dental resin, a filler, and the protein-repellant agent.

121. The protein-repellant dental material of claim 116, wherein the protein-repellant dental material further comprises an antibacterial agent, a remineralizing agent, or both.

122. The protein-repellant dental material of claim 117, wherein the dental primer comprises one or more primers selected from the group consisting of bisphenol A diglycidyl methacrylate (Bis-GMA), glycerol dimethacrylate (GDMA), 2-hydroxyethyl methacrylate (HEMA), mono-2-methacryloyloxyethyl phthalate (MMEP), methacrylic acid (MA), methyl methacrylate (MMA), 4-acryloyloxyethyl trimellitate anhydride (4-AETA), N-phenylglycine glycidyl methacrylate (NPG-GMA), N-tolylglycine glycidyl methacrylate or N-(2-hydroxy-3-((2-methyl-1-oxo-2-propenyl)oxy)propyl)-N-tolyl glycine (NTG-GMA), pyromellitic diethylmethacrylate or 2,5-dimethacryloyloxyethyloxycarbonyl-1,4-benzenedicarboxylic acid (PMDM), pyromellitic glycerol dimethacrylate or 2,5-bis(1,3-dimethacryloyloxyprop-2-yloxycarbonyl)benzene-1,4-dicarboxylic acid (PMGDM), and triethylene glycol dimethacrylate (TEGDMA).

123. The protein-repellant dental material of claim 117, wherein the amount of protein-repellant agent in the protein-repellant dental material ranges from about 5% to about 12.5% of the mass of the dental material.

124. The protein-repellant dental material of claim 118, wherein the dental adhesive comprises one or more adhesives selected from the group consisting of ethoxylated bisphenol A glycol dimethacrylate (Bis-EMA), bisphenol A diglycidyl methacrylate (Bis-GMA), 2-hydroxyethyl methacrylate (HEMA), triethylene glycol dimethacrylate (TEGDMA), urethane dimethacrylate (UDMA), 4-methacryloyloxyethyl trimellitate anhydride (4-META), methacrylic acid (MA), methyl methacrylate (MMA), 4-acryloyloxyethyl trimellitate anhydride (4-AETA), ethyleneglycol dimethacrylate (EGDMA), glycerol dimethacrylate (GDMA), glycerol phosphate dimethacrylate (GPDM), pyromellitic glycerol dimethacrylate or 2,5-bis(1,3-dimethacryloyloxyprop-2-yloxycarbonyl)benzene-1,4-dicarboxylic acid (PMGDM).

125. The protein-repellant dental material of claim 118, wherein the amount of protein-repellant agent in the protein-repellant dental material ranges from about 5% to about 12.5% of the mass of the dental material.

126. The protein-repellant dental material of claim 119, wherein the dental resin comprises one or more resins selected from the group consisting of bis-GMA (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer, and a heat-cured polymer.

127. The protein-repellant dental material of claim 119, wherein the amount of protein-repellant agent in the protein-repellant dental material ranges from about 2.5% to about 10% of the mass of the dental material.

128. The protein-repellant dental material of claim 120, wherein the dental resin comprises one or more resins selected from the group consisting of bis-GMA (bisphenol glycidyl methacrylate), TEGDMA (triethylene glycol dimethacrylate), HEMA (2-hydroxyethyl methacrylate), UDMA (urethane dimethacrylate), PMGDM (pyromellitic acid glycerol dimethacrylate), ethoxylated bisphenol A dimethacrylate (EBPADMA), methacryloyloxyethyl phthalate (MEP), methacrylate-modified polyalkenoic acid, a hydrophobic monomer, a hydrophilic monomer, a poly acid-modified polymer, a light-cured polymer, a self-cured polymer, a duel cured polymer, and a heat-cured polymer.

129. The protein-repellant dental material of claim 120, wherein the amount of protein-repellant agent in the protein-repellant dental material ranges from about 2.5% to about 10% of the mass of the dental material.

130. The protein-repellant dental material of claim 121, wherein the antibacterial agent is one or more of antibacterial monomers, silver-containing nanoparticles (NAg), quaternary ammonium salts (QAS), chlorhexidine particles, TiO₂ particles and ZnO particles.

131. The protein-repellant dental material of claim 130, wherein the antibacterial monomers are selected from the group consisting of dimethylamino propyl methacrylate (DMAPM), dimethylamino hexyl methacrylate (DMAHM), dimethylamino heptyl methacrylate (DMAHPM), dimethylamino octyl methacrylate (DMAOM), dimethylamino nonyl methacrylate (DMANM), dimethylamino decyl methacrylate (DMADM), dimethylamino undecyl methacrylate (DMAUDM), dimethylamino dodecyl methacrylate (DMADDM), dimethylamino tridecyl methacrylate (DMATDM), dimethylamino tetradecyl methacrylate (DMATTDM), dimethylamino pentadecyl methacrylate (DMAPDM), dimethylamino hexadecyl methacrylate (DMAHDM), dimethylamino heptadecyl methacrylate (DMAHPDM), dimethylamino octadecyl methacrylate (DMAODM), dimethylamino nonadecyl methacrylate (DMANDM), dimethylamino icosyl methacrylate (DMAIOM), dimethylamino henicosyl methacrylate (DMAHOM), and dimethylamino docosyl methacrylate (DMADOM).

132. The protein-repellant dental material of claim 130, wherein the amount of antibacterial monomers present in the protein-repellant dental material ranges from about 2.5% to about 12.5% of the mass of the dental material.

133. The protein-repellant dental material of claim 121, wherein the remineralizing agent is nanoparticles of amorphous calcium phosphate (NACP).

134. The protein-repellant dental material of claim 133, wherein the amount of NACP present in the protein-repellant dental material ranges from about 10% to about 40% of the mass of the dental material.

135. The protein-repellant dental material of claim 117, wherein MPC is present in an amount ranging from about 5-10% by mass of the dental material.

136. The protein-repellant dental material of claim 118, wherein MPC is present in an amount ranging from about 5-10% by mass of the dental material.

137. The protein-repellant dental material of claim 121, wherein the dental material is a protein-repellant dental resin comprising a dental resin, MPC, and an antibacterial agent, wherein MPC is present in an amount ranging from about 5-10% by mass of the dental resin, wherein the antibacterial agent is present in an amount ranging from about 2.5-7.5% by mass of the dental material, and wherein the antibacterial agent is DMADDM, DMAPDM, or DMAHDM.

138. A protein-repellant dental material of claim 121, wherein the dental material is a protein-repellant dental composite comprising a dental resin, a glass filler, MPC, and an antibacterial agent, wherein glass filler is present in an amount ranging from about 60-80% by mass of the dental composite, wherein MPC is present in an amount ranging from about 2-5% by mass of the dental composite, wherein the antibacterial agent is present in an amount ranging from about 1-5% by mass of the dental material, and wherein the antibacterial agent is DMADDM, DMAPDM, or DMAHDM.
